(19) United States
(12) Patent Application Publication
(10) Pub. No.: US 2025/0172542 A1
(43) Pub. Date: May 29, 2025

(54) METHOD FOR INDUCING ARTIFICIAL AGEING IN A STEM CELL AND USES OF SAID CELL

(71) Applicant: Clock Bio Limited, Cambridge (GB)

(72) Inventors: Mark Kotter, Cambridge (GB); Koby Baranes, Cambridge (GB)

(21) Appl. No.: 18/859,587

(22) PCT Filed: Apr. 27, 2023
(86) PCT No.: PCT/GB2023/051123

§ 371 Date: Oct. 24, 2024

(30) Foreign Application Priority Data
Apr. 27, 2022 (GB) 2206113.9

(51) Int. Cl. G01N 33/50

(52) U.S. Cl. CPC G01N 33/5023

(57) ABSTRACT

The present invention relates to methods of transiently inducing ageing in a stem cell or a somatic cell derived from an induced pluripotent stem cell (iPSC) by forward programming, said methods comprising introducing one or more ageing-inducing factor into the cell or exposing the cell to one or more exogenous ageing-inducing factor for a period of time, such as prior to or after forward programming of the iPSC, followed by removing or reducing the one or more ageing-inducing factor, thereby leading to a reversal of one or more ageing phenotype in the cell. Also provided is an artificially aged stem cell or somatic cell derived from an iPSC by forward programming, and its use in a method of identifying a gene or a combination of genes involved in the reversal of an ageing phenotype or in the maintenance of a non-aged phenotype in the cell. A method of identifying a gene or combination of genes involved in the reversal of an ageing phenotype or in the maintenance of a non-aged phenotype in a cell is also provided, said method comprising introducing one or more ageing-inducing factor into the cell or exposing the cell to one or more exogenous ageing-inducing factor for a period of time and measuring an ageing phenotype of the cell. In particular, the methods of identifying a gene or a combination of genes involved in the reversal of an ageing phenotype or in the maintenance of a non-aged phenotype in the cell optionally comprise performing a loss-of-function, inhibitory, knock-out, gain-of-function or combinatorial screen. Further provided is a method of rejuvenating a somatic cell comprising altering the expression and/or activity of one or more gene identified by the methods described herein.

### FIELD OF THE INVENTION

The present invention relates to methods of transiently inducing ageing in a stem cell or a somatic cell derived from an induced pluripotent stem cell (iPSC) by forward programming, said methods comprising introducing one or more ageing-inducing factor into the cell or exposing the cell to one or more exogenous ageing-inducing factor for a period of time, such as prior to or after forward programming of the iPSC, followed by removing or reducing the one or more ageing-inducing factor, thereby leading to a reversal of one or more ageing phenotype in the cell. Also provided is an artificially aged stem cell or somatic cell derived from an iPSC by forward programming, and its use in a method of identifying a gene or a combination of genes involved in the reversal of an ageing phenotype or in the maintenance of a non-aged phenotype in the cell. A method of identifying a gene or combination of genes involved in the reversal of an ageing phenotype or in the maintenance of a non-aged phenotype in a cell is also provided, said method comprising introducing one or more ageing-inducing factor into the cell or exposing the cell to one or more exogenous ageing-inducing factor for a period of time and measuring an ageing phenotype of the cell. In particular, the methods of identifying a gene or a combination of genes involved in the reversal of an ageing phenotype or in the maintenance of a non-aged phenotype in the cell optionally comprise performing a loss-of-function, inhibitory, knock-out, gain-of-function or combinatorial screen. Further provided is a method of rejuvenating a somatic cell comprising altering the expression and/or activity of one or more gene identified by the methods described herein.

### BACKGROUND OF THE INVENTION

Ageing is the shared risk factor of a multitude of diseases, ranging from cancer to dementia (Niccoli and Partridge (2012) Curr. Biol.). However, a number of findings demonstrated that the ageing trajectory can be modulated, e.g. by dietary interventions (Longo and Mattson (2014) Cell Metab.; Longo et al. (2021) Nat. Ageing), drugs (Kennedy and Pennypacker (2014) Tranl. Res.; Guilbert et al. (2021) Methods), and genetic manipulation (Simpson et al. (2021) Clin Epigenetics; Zhang et al. (2020) Nat Rev Mol Cell Biol). Most importantly, reprogramming of somatic cells into induced pluripotent stem cells is able to reverse many hallmarks of ageing (Lapasset et al. (2011) Genes Dev.). Hence, there exist mechanisms that are able to modulate ageing, which deserve further investigation.

Whilst the process of ageing is still poorly understood, it affects cells in multiple ways. Otin et al. (2013) Cell have provided a comprehensive review of “The Hallmarks of Aging”, which include mitochondrial changes, senescence, altered intracellular communication, genomic instability, telomere shortening, epigenetic changes, and deregulation of nutrient pathways. These may lead to changes in cellular function, such as a reduced ability of immune cells to survey cancerogenic events, which ultimately contribute to the plethora of age-related diseases.

Stem cells provide scalable model systems to study the biology of a species. However, modelling late-onset disorders using pluripotent stem cells (PSCs) such as embryonic stem cell (ESC) and induced pluripotent stem cell (iPSC) technology holds a challenge due to the embryonic nature of pluripotent stem cells and in particular the cellular rejuvenation during iPSC reprogramming (Lapasset et al. (2011) Genes Dev.; Maherali et al. (2007) Cell Stem Cell; Studer et al. (2015) Stem Cell, 16:591-600).

Moreover, hallmarks of aging are often related to disease associated degenerative processes. Independently, they have been associated with a gradual deterioration in structure and function. Some of these hallmarks have been identified as nuclear blebbing and folding, shortening of dendrite length (in the case of neurons), telomere attrition, reduced proteostasis and the accumulation of DNA damage and mitochondrial reactive oxygen species (ROS) (Lopez-Otin et al. (2013) Cell; Miller et al. (2013) Cell Stem Cell; Studer et al. (2015) Stem Cell). In addition, changes in gene expression profile have been observed in neurons (Arancio (2019) GeroScience; Mertens et al. (2015) Cell Stem Cell, 17:705-718) and in the human brain in general (Dillman et al. (2017) Sci. Rep.).

These findings further corroborate the need for generating faithful iPSC models of human disease and have therefore inspired two main strategies to induce cellular aging. The first approach involves manipulating the cellular environment by introducing toxic stressors such as reactive oxygen species (Davalli et al. (2016) Oxid. Med. Cell. Longev.), or by inflammatory cytokines to mimic the general overall inflammatory state associated with aging. The second approach involves introducing intrinsic changes to the cells to induce aging. That was accomplished by promoting telomere shortening through pharmacological inhibition of telomerase, the enzyme involved in maintaining telomere length (Vera et al. (2016) Cell Rep.). In this study, similar age- and disease-related phenotype changes such as an increase in mitochondrial ROS production, DNA damage and loss tyrosine hydroxylase expression were reported in Parkinsonian midbrain dopaminergic neurons. However, the study remains preliminary, as the effect on other cellular processes was not looked at.

There is therefore a need to develop systematic methods to identify genes and regulatory networks that contribute to ageing and the reversal of ageing in iPSCs which can subsequently be applied to somatic cells.

### SUMMARY OF THE INVENTION

According to a first aspect of the invention, there is provided a method of transiently inducing ageing in a stem cell, said method comprising:

(i) introducing one or more ageing-inducing factor into the cell or exposing the cell to one or more exogenous ageing-inducing factor for a period of time, and

(ii) removing or reducing the one or more ageing-inducing factor, thereby leading to a reversal of one or more ageing phenotype in the cell.

According to a further aspect of the invention, there is provided a method of transiently inducing ageing in a somatic cell derived from an induced pluripotent stem cell (iPSC) by forward programming, said method comprising:
(i) introducing one or more ageing-inducing factor into the cell or exposing the cell to one or more exogenous ageing-inducing factor for a period of time prior to or after forward programming of the iPSC, and
(ii) removing or reducing the one or more ageing-inducing factor, thereby leading to a reversal of one or more ageing phenotype in the cell.

In certain embodiments, removing or reducing the one or more ageing-inducing factor in step (ii) comprises active removal or reduction, thereby leading to the reversal of one or more ageing phenotype in the cell.

In a further aspect, there is provided an artificially aged stem cell or somatic cell derived from an iPSC by forward programming produced by the methods described herein.

In a yet further aspect, there is provided a use of the artificially aged stem cell and/or somatic cell derived from an iPSC by forward programming as defined herein in a method of identifying a gene or a combination of genes involved in the reversal of an ageing phenotype or in the maintenance of a non-aged phenotype in the cell.

According to another aspect of the invention, there is provided a method of identifying a gene or a combination of genes involved in the reversal of an ageing phenotype or in the maintenance of a non-aged phenotype in a cell, said method comprising the steps of:
i) introducing one or more ageing-inducing factor into the cell or exposing the cell to one or more exogenous ageing-inducing factor for a period of time;
ii) optionally performing a loss-of-function, inhibitory, knock-out, gain-of-function or combinatorial screen in the cell;
iii) measuring an ageing phenotype of the cell; and
iv) identifying a gene or a combination of genes as being involved in the reversal of an ageing phenotype or in the maintenance of a non-aged phenotype when the ageing phenotype is altered and/or its progression is altered in the cell when a loss-of-function, inhibitory, knock-out, gain-of-function or combinatorial screen is performed compared to when the cell is not subjected to a screen.

In another aspect of the invention, there is provided a method of rejuvenating a somatic cell comprising altering the expression and/or activity of one or more gene identified by the methods described herein.

### BRIEF DESCRIPTION OF THE FIGURES

FIG. 1: Ageing and Rejuvenation of Aged hiPSCs. A) Schematic of experimental procedure to induce aged-iPSCs and aged cortical glutamatergic neurons that were “aged” either pre-(i) or post-(ii) conversion of the iPSCs into neurons. B-C) hiPSC lines were generated in two genetic backgrounds, overexpressing Progerin only under doxycycline control (B) and cells were targeted with NGN2 and progerin inducible cassettes for overexpression in a dual system using the TetOn and Cumate systems (C). D-F) NGN2 iPSCs (D), NGN2-Progerin iPSCs (E) and Progerin iPSCs (F) were cultured with or without cumate or dox to overexpress progerin and artificially age cells. Aged iPSCs retain their pluripotency markers (i.e. Nanog and OCT4) after inducing GFP-progerin for 5 days. However, lamin-A and GFP staining revealed abnormal nuclear membrane morphologies in the aged iPSCs. Following passage of the aged iPSCs and discontinuation of progerin expression, the cells restore their morphology (E and F, 1^{st} and 2^{nd} splits). G-H) Nuclei of aged NGN2-Progerin iPSCs (G) and aged Progerin iPSCs (H) were found to be less circular and more elongated compared to non-aged iPSCs. Aged iPSCs in which progerin is no longer expressed reverse their morphology abnormalities following 1^{st} passage/split. Data is presented as means±SEM, one-way Anova test. Scale bars=25 μm. NGN−, no cumate; NGN+, with cumate; N2P−, no cumate; N2P+, with cumate; 1^{st}/2^{nd} split, aged N2P/Progerin iPSCs 1^{st} or 2^{nd} split post ageing. * p<0.05, ** p<0.01, *** p<0.001, **** p<0.0001.

FIG. 2: Morphological changes following overexpression of progerin in KOLF Progerin iPSCs. A) Progerin iPSCs were cultured with or without dox to overexpress progerin. Aged iPSCs retain their pluripotency markers Nanog and OCT4 after inducing GFP-progerin for 5 days. GFP staining revealed abnormal nuclear membrane morphologies for the aged iPSCs. Following passage of the aged iPSCs and when progerin expression was discontinued, the cells restore their morphology. Scale bars=25 μm. B) Morphological analysis of nucleus circularity, roundness and size following overexpression of progerin and after 1^{st} and 2^{nd} splits post ageing. Nucleus circularity and roundness were decreased in the aged cells, but spontaneously returned to normal morphologies after the 1^{st} split post ageing, when progerin expression was discontinued. Interestingly, the nucleus size was also significantly reduced following ageing. Data is presented as means±SEM, one-way Anova test. ** p<0.01, **** p<0.0001.

FIG. 3: Reduced heterochromatin expression and increased DNA damage aged iPSCs. A-C) Marker for peripheral heterochromatin H3K9me3 was significantly downregulated after inducing ageing with cumate in hiPSCs. However, after 1 passage post discontinuation of progerin expression, cells start to show recovery in H3K9me3 expression. Scale bars=25 μm. Data is presented as means±SD, one-way Anova test. D-E) H3K9me3 was significantly reduced in the aged iPSCs after overexpression of progerin by dox but was back to normal levels after 1^{st} and 2^{nd} splits post ageing. Scale bars=25 μm. Data is presented as means±SD, one-way Anova test. F) Aged iPSCs display double strand DNA breaks (based on γH2AX staining), which are no longer present after the 1^{st} split post ageing. Scale bars=10 μm. G) The number of foci based on γH2AX expression was counted. Aged iPSCs showed the highest number of foci ≥5, while non aged cells and those aged by transient progerin expression after the 1^{st} and 2^{nd} splits were significantly lower. Data is presented as means±SEM, one-way Anova test. NGN−, no cumate; NGN+, with cumate; N2P−, no cumate; N2P+, with cumate; 1^{st}/2^{nd} split, aged N2P/Progerin iPSCs 1^{st} or 2^{nd} split post ageing. * p<0.05, ** p<0.01, *** p<0.001, **** p<0.0001, ns=not significant.

FIG. 4: Immunostaining for heterochromatin and DNA damage markers in Kolf aged iPSCs. A) According to Immunocytochemistry, the expression of the heterochromatin peripheral marker, H3K9me3, seems to be reduced in the transiently aged iPSCs, but is back to normal levels after 1^{st} and 2^{nd} splits post ageing. Scale bars=25 μm. B) Aged iPSCs display double strand DNA breaks (based on γH2AX staining), which reduce after the 1^{st} split post ageing. Scale bars=10 μm. C) Quantitative analysis of H3K9me3 expression. Data is presented as means±SEM, one-way Anova test. D) The number of foci based on γH2AX expression was counted. Aged iPSCs showed the highest number of foci ≥5, while non aged and transiently aged iPSCs after the 1^{st} and 2^{nd} splits were significantly lower. Data is presented as means±SEM, one-way Anova test. ** p<0.01, **** p<0.0001, ns=not significant.

FIG. 5: Senescence in aged iPSCs. A) Phase images of Bob and Kolf non-aged and aged iPSCs following β-galactosidase (β-gal) staining. Scale bars=250 μm. B) Based on quantitative analysis, aged iPSCs showed a significant increase in β-gal staining compared to non-aged iPSCs. Data is presented as means±SD, one-way Anova test. * p<0.05, ** p<0.01.

FIG. 6: Initial NGN2-Progerin construct used by the OPTi-OX system to generate aged iNeurons. A) Initial NGN2-Progerin reprogramming cassette for overexpressing both NGN2 and progerin under the control of TetOn system. NGN2 and GFP-Progerin are separated by P2A site and are expressed at the same time. B) Phase and confocal images of non-aged (iNs) and aged iNeurons (iaNs) 3 days post induction. GFP-Progerin expression is visible (left panels). Nuclear folding and blebbing is observed by Lamin-A staining (right panels). Scale bars=25 μm. C) Upregulation of Lamin-A and C following overexpression of progerin in the aged iNeurons, based on RT-PCR analysis. D) Aged iNeurons show reduction in mitochondrial function based on oxygen consumption rates (OCRs) measurements E) Viability assay at day 1 and day 3 post induction shows massive cell death of the aged iNeurons.

FIG. 7: Development of a dual inducible system, (OPTi-OX) 2, to artificially age iPSCs and iNeurons. A) Workflow for targeting the human ROSA26 and AAVS1 loci with the Tet-ON and Cumate systems in hiPSCs for inducible NGN2/NGN2-Progerin expression. Cas9n, D10A nickase mutant Cas9 endonuclease; ZFN, zinc-finger nucleases; rtTA, reverse tetracycline transactivator; CymR, cumate repressor. B) Schematic of the four outcomes following generation of dual GSH-targeted inducible NGN2/NGN2-Progerin hiPSCs, either in the OptiOX (Tet-On only) or (OptiOX)² (Tet-ON/Cumate) systems: clonal lines were categorized based on the number of successfully targeted alleles of the human ROSA26 and AAVS1 loci. C) Two cassettes were designed, the first construct orientates NGN2 and progerin transgenes in the same way, whilst the second construct orients both transgenes in opposite directions to minimise potential leakage. D) Genotyping results for selected human ROSA26-CAG-rtTA-CymR targeted (left) and AAVS1-NGN2-Progerin re-targeted hiPSCs (right). Heterozygous (for the ROSA26 locus) and homozygous (for the AAVS1 locus) lines for each allele are shown. WT: wild-type hiPSCs; B.N2P: Bob NGN2-Progerin; K.N2P, KOLF2 NGN2-Progerin; NT: no template control. E) Phase images of the 3 lines tested (Bob NGN2, Bob NGN2-Progerin, KOLF2 NGN2-Progerin) after 7 days of induction. No differences in neuronal growth were observed between the lines. Scale bars=100 μm. F-G) Phase images (F) and immunocytochemistry (G) of 1-week post induction non-aged (dox only) and aged iNeurons (dox+cumate). Folding and blebbing of nuclear membrane is observed by GFP-Progerin expression following overexpression of progerin at different concentrations. Scale bars=50 μm upper panels; 10 μm lower panels. H) Cumate dose response assay. Data is presented as means±SD, one-way Anova test. I) Viability assay. Data is presented as means±SD, Student t-test.

FIG. 8: Pre- and post-aged iNs exhibit nuclear morphology abnormalities. A-B) Co-localization of GFP-Progerin and lamin-A expression in both Bob-N2P and KOLF-N2P aged iNeurons for D14 (A) and D21 (B) post induction, shows folded and blabbed nuclear morphologies. No expression was observed for the ctrl iNeurons (Bob-NGN2). Scale bars: left panels=25 μm; right panels=10 μm. C-D) Lamina associated protein, LAP2α, was found to be significantly downregulated following overexpression of progerin at D14 (C) and D21 (D) post induction. Scale bars=25 μm. E-F) Quantitative analysis of LAP2α expression at D14 (E) and D21 (F) post induction. Data is presented as mean±SEM. One-way Anova test. G-H) Nucleus circularity and roundness were found to be affected in the aged iNeurons compared to non-aged ones at D14 (G) and D21 (H) post induction. Data is presented as mean±SEM. One-way Anova test. * p<0.05, ** p<0.01, *** p<0.001, **** p<0.0001, ns=not significant.

FIG. 9: Degeneration of neurites following overexpression of progerin. A) Overexpression of progerin leads to reduced intact dendrite length in aged iNeurons (iaNs/aiNs) compared to non-aged iNeurons (iNs) at D7 (top panel), D14 (middle panels), and D21 (lower panels) post induction, based on immunocytochemistry for β3-tubulin and MAP2. Scale bars=100 μm phase, 25 μm ICC. B-C) Aged iNeurons (iaNs/aiNs) had significantly shorter dendrites compared to non-aged iNeurons at both D14 (B) and D21 (C) post induction, based on MAP2 staining quantitative analysis. Data is presented as mean±SEM, one-way Anova. D-E) D21 aged iNeurons diameter (D) was significantly lower with narrow distribution of neurites diameter (E) centred around 300 nm compared to non-aged iNeurons which had broader distribution. Non-linear regression, Gaussian distribution. Means±SEM, one-way Anova. F) mRNA levels of cytoskeleton (β3-tubulin, MAP2) and synapse (Synaptophysin, synapsyin-1, PSD95, vGlut2) related neuronal markers are downregulated after inducing ageing in iNeurons. Data is presented as mean±SD, all data relative to PBGD1, one-way Anova. G) Synapsin-1 was downregulated following overexpression of progerin based on immunocytochemistry. Scale bars=25 μm. * p<0.05, ** p<0.01, *** p<0.001, **** p<0.0001, ns=not significant.

FIG. 10: Progerin by itself is responsible for the ageing phenotype and not cumate. A) Comparison between Bob NGN2 and Bob N2P iNs treated with or without cumate. RT-PCR analysis verified cumate was not responsible for the changes seen in gene expression, i.e. MAP2 and synapsin-1 downregulation. Data is presented as means±SD, all data relative to PBGD1, student t-test. B) Immunocytochemistry of Bob N2P and Bob NGN2 iNeurons treated with (middle and lower panels) or without (upper panel) cumate. Overexpression of progerin led to reduction in dendritic morphology and H3K9me3 expression. Scale bars=25 μm. ** p<0.01, ns=not significant.

FIG. 11: Peripheral heterochromatin markers expression and DNA damage in aged iNeurons. A-B) Overexpression of progerin in aged iNeurons (iaNs/aiNs) leads to decrease in H3K9me3 (A) and HP1γ (B) expression at D14 (A-B, upper panels) and D21 (A-B, lower panels) post induction. Scale bars=25 μm. C-F) H3K9me3 and HP1γ are significantly downregulated at D14 (C, E) and D21 (D, F) post induction compared to control non-aged iNeurons. Data is presented as means±SEM, one-way Anova. G) DNA damage, based on γH2AX staining, was observed for aged iNeurons from both lines (Bob and Kolf) following overexpression of progerin. Scale bars=15 μm. H-I) The number of foci>5 per cell was higher in the aged iNeurons group, which had 18% and 12% cells with ≥3 foci for the Bob and Kolf lines, respectively. * p<0.05, ** p<0.01, *** p<0.001, **** p<0.0001, ns=not significant.

FIG. 12: Mitochondrial function and electrophysiology in the aged iNeurons. A-C) The Oxygen consumption rates (OCRs) were measured for D14 non-aged (iNs) and aged (iaNs/aiNs) iNeurons. Both post-(A-B) and pre-aged (B) iNeurons show reduction in mitochondrial function compared to non-aged iNeurons. Data is presented as means±SD. D) Basal respiration levels were significantly lower in the aged groups compared to the non-aged group. Data is presented as means±SD. One-Way Anova. E) Viability assay following OCRs measurement confirmed the reduction in OCRs was due to progerin overexpression and not due to cell death. Live cells normalized to the control (Bob NGN2 or Bob N2P iNeurons treated with dox only). F) Schematic of the multi electrode array (MEA) (Axion) used in this study. Each well contains 16 electrodes. G) Electrophysiological activity recordings on MEA's of non-aged iNeurons (iNs) and aged iNeurons (iaNs/aiNs) with and without astrocytes. The mean firing rate was significantly lower for the aged group at D14 post induction, however by D21 the activity resembled that of the non-aged group. Data is presented as means±SEM. One-Way Anova. H) No differences in burst duration were observed in the aged group compared to the non-aged group at D21 post induction, suggesting no effect on neuronal network communication. Data is presented as means±SEM. One-Way Anova. ****p<0.0001, ns=not significant.

FIG. 13: Transcriptomic profile of aged cells following overexpression of progerin. A) Principal component (PC) analysis demonstrated tight clustering of the control non-aged iPSCs (middle panel) group and iNeurons (right panel) group. B) Overlap of differentially expressed genes (two left panels) and ageing related genes (two right panels). The analysis provides a list of genes which can be used for CRISPR screening to identify gene regulatory networks that contribute to ageing phenotypes. Indicated in the diagram are the numbers of upregulated/downregulated DEGs. C) Heatmap representation of relevant Gene-Ontology (GO) terms based on differential expression from bulk RNA sequencing.

FIG. 14: GFP and progerin counts based on RNA sequencing reads. The aged group show higher number of reads for both GFP (top panel) and progerin (bottom panel, while the control non-aged group has no reads. However, for Bob N2P line, the data suggest of a leak from the cumate inducible system, as expression of GFP was detected in iPSCs not treated with cumate (iPSCs DO N2P).

FIG. 15: A) Expression of iPSCs marker genes in aged vs non-aged iPSCs shows downregulation of Nanog and OCT4 (POU5F1). B) Volcano plot for the iPSCs marker genes in aged vs non-aged iPSCs.

FIG. 16: Gene ontology (GO) terms analysis. The aged iNeurons group showed downregulation in GO-terms related to synaptic transmission, action potential and morphogenesis and upregulation of GO-terms related to DNA damage, apoptosis and senescence at D14 and D21 post induction.

FIG. 17: Epigenetic clock analysis in the aged iPSCs and iNeurons. A) Transcriptomic clock analysis based on Tabula Muris Senis (mouse ageing cell atlas). The ‘non-zero clock’ and the ‘binarized’ clock are both trained using Bulk RNA-seq data, revealing that there are also epigenetic effects in the aged iNeurons. However, these clocks do not work well with predicting iPSCs age, as the data is compared to a mouse brain. B) Epigenetic clock analysis base on Steve Horvath clock (Horvath 2013 Genome Biol). No significant differences were observed between non-aged and aged iPSCs/iNeurons, although there seems to be a shift towards an older age for the aged group. Bob N2P iPSCs treated with cumate show similar results as non-treated ones, supporting the possibility of a leak in the cumate inducible system. C) DNA methylation entropy analysis based on the number of methylated CpG serves as a good prediction for ageing. Both Bob N2P iPSCs (left panel) and iNeurons (right panel) treated with cumate show higher entropy than those generated from Bob NGN, suggesting of an epigenetic ageing. ** p<0.01, *** p<0.001. +cumate, 75 μg/mL cumate; ++cumate, 150 μg/mL cumate.

FIG. 18: Horvath epigenetic analysis suggests of a shift toward CD4 T cells. A) All aged lines (post- and pre-aged) displayed heterogeneous population of neuronal and non-neuronal-like cells after 14 days in culture. These non-neuronal cells do not appear until overexpression of progerin was initiated at D7. Scale bars=100 μm. B) Epigenetic analysis by Horvath suggests of neurons losing their neuronal identity and shifting towards CD4 T cells. 75=75 μg/mL cumate; 150=150 μg/mL cumate. C) Heatmap for specific cell type markers (e.g. pluripotency, neuronal, T cells) showed no correlation between the aged iNeurons and T cells.

FIG. 19. CRISPR screens to identify target genes involved in ageing and rejuvenation. A) Engineering of a Cas9 line based on the progerin model. Expressions of progerin (GFP) and Cas9 (mCherry) were observed (right-hand panels). Cas9 KO efficiency was verified by loss of B2M protein expression upon delivery of the specific sgRNA (red, unfilled) compared to an untreated control (grey, filled), thus confirming Cas9 activity in this cell line. B) Schematic representation of the crop-sequencing screen based on single cell RNA-sequencing readout. C) Number of cells per knockout which were identified. D) A linear discriminant analysis (LDA) using the ageing signature was used to create an ageing scale. This scale integrated into a Uniform Manifold Approximation and Projection (UMAP) plot of all single cells processed within the CRISPR knockout screen. E) EZH2 gene was identified as having an anti-ageing effect based on this ageing scale (left curve=EZH2 targeting, right curve=non-targeting control).

### DETAILED DESCRIPTION OF THE INVENTION

According to a first aspect of the invention, there is provided a method of transiently inducing ageing in a stem cell, said method comprising:
(i) introducing one or more ageing-inducing factor into the cell or exposing the cell to one or more exogenous ageing-inducing factor for a period of time, and
(ii) removing or reducing the one or more ageing-inducing factor, thereby leading to a reversal of one or more ageing phenotype in the cell.

The terms “age” and “ageing” as used herein refer to the phenotypic changes associated with age which restrict functionality and can be considered to be a consequence of ageing. These ageing phenotypes can be distinct from ageing DNA correlates such as epigenetic changes and double strand breaks for which the causality with ageing has not yet been established and reversal of which may not lead to a reversal of ageing or maintenance of a non-aged phenotype. However, it will be appreciated that these correlates may be measured in the methods described herein as alternative/further ageing phenotypes. Examples of cellular phenotypes associated with ageing are: mitochondrial dysfunction, senescence, altered intracellular communication, genomic instability, telomere shortening, epigenetic changes, and deregulation of nutrient pathways (Otin et al. (2013) Cell “The Hallmarks of Aging”). Other ageing phenotypes include: reduced proliferation, changes in cell and/or nuclear morphology, changes in gene expression, and upregulation of Lamin-A and/or Lamin-C nuclear protein. Thus, “age” and “ageing” may refer to any ageing phenotype herein and in certain embodiments refer to the phenotype of the cell (e.g. the ageing phenotype), such as the phenotype of the cell measured as part of a method of identifying a gene or combination of genes involved in the reversal or maintenance of a non-aged phenotype as described herein.

In one embodiment, the ageing phenotype is selected from one or more of: proliferation, senescence, changes in cell and/or nuclear morphology, mitochondrial function, changes in gene expression, upregulation of Lamin-A and/or Lamin-C nuclear protein, epigenetic marks associated with ageing, altered methylation entropy, DNA double strand breaks, telomere length, and a transcriptomic and/or epigenetic clock. In a further embodiment, the ageing phenotype is reduced proliferation and/or increased senescence. In another embodiment, the changes in nuclear morphology are folding abnormalities, blebbing and/or loss of nuclear circularity. In a yet further embodiment, the mitochondrial function is reduced oxygen consumption and/or increased mitochondrial reactive oxygen species (ROS). In a still further embodiment, the changes in gene expression are selected from one or more of: downregulation of somatic cell lineage identity genes, downregulation of mitochondrial genes, upregulation of apoptosis- and/or senescence-related genes, and upregulation of DNA damage-related genes. In another embodiment, the epigenetic marks associated with ageing are selected from one or more of: reduced heterochromatin trimethylated H3K9 (H3K9me3), reduced HP1γ, and increased γH2AX. In a further embodiment, the telomere length is shortened. In a yet further embodiment, the transcriptomic and/or epigenetic clock is a single cell transcriptomic and/or epigenetic clock. In a certain embodiment, the ageing phenotype is a transcriptomic clock, such as a transcriptomic signature associated with an aged phenotype. In a further embodiment, the ageing phenotype is a single cell transcriptomic clock, such as a transcriptomic signature associated with an aged phenotype. In a still further embodiment, the ageing phenotype, such as the ageing phenotype of the cell, is a combination of one or more of the ageing phenotypes described herein.

In one embodiment, the “age” of the cell or whether the cell is “aged” is determined using an epigenetic clock, such as the Horvath epigenetic clock (Horvath, S (2013) Genome Biol., doi: https://doi.org/10.1186/gb-2013-14-10-r115). Thus, in a further embodiment inducing ageing in a cell comprises ageing the cell according to an epigenetic clock, such as the Horvath epigenetic clock. The Horvath epigenetic clock can be used as an age estimation method based on DNA methylation at CpG dinucleotide motifs in the DNA. DNA methylation age (further known as a “predicted age”) is characterised by the following properties: it is close to zero for ES and iPS cells; it correlates with cell passage number; it gives rise to a highly heritable measure of age acceleration; and it is applicable to chimpanzee tissues. The DNA methylation age of blood has been shown to predict all-cause mortality in later life, even after adjusting for known risk factors, suggesting that it is related to processes that cause ageing. Similarly, markers of physical and mental fitness have been associated with the epigenetic clock. One particular feature of the Horvath epigenetic clock is its high accuracy and applicability to a broad spectrum of tissues and cell types. Since it allows one to contrast the ages of different tissues and cells from the same subject (including a forward programmed somatic cell with a non-iPSC derived somatic cell of the same lineage or a pluripotent cell, such as an iPSC), it can be used to identify tissues and cells that show evidence of accelerated age due to disease. Furthermore, the Horvath epigenetic clock may be used to identify any change in DNA methylation age caused by treatment, such as reprogramming or forward programming.

In another embodiment, “age” is determined using a transcriptomic clock, such as a single cell transcriptomic clock. Thus, in a further embodiment inducing ageing in a cell comprises ageing the cell according to a transcriptomic clock. In some embodiments, the transcriptomic clock comprises a transcriptomic signature associated with an aged phenotype of the cell. One example of transcriptomic clocks is described in Fleischer et al. (2018) Genome Biol. 19, 221 (doi: http://doi.org/10.1186/s13059-018-1599-6). Previously known ageing clocks trained on single cell RNA sequencing signatures typically suffer from: i) restriction to particular cell types; ii) lower accuracy than epigenetic-based ageing clocks; and iii) high variance of age measurements. However, these may be overcome in the methods described herein using a novel bioinformatic approach which yields single cell transcriptomic clocks that: i) are cell type independent; ii) exhibit accuracy rivalling epigenetic ageing clocks; and iii) have minimal variance in age measurements. Said bioinformatic approach may also be applied to multiomic data where data sets such as genomic, proteomic and epigenomic data sets are combined. In a further embodiment, a transcriptomic clock, such as a single cell transcriptomic clock, may be combined with any ageing phenotype described herein. For example, in some embodiments age is determined using a combination of a single cell transcriptomic clock with changes in cell and/or nuclear morphology, epigenetic clock measurements, mitochondrial function, epigenetic marks associated with ageing, altered methylation entropy, gene expression or any combination thereof.

Inducing ageing according to an epigenetic and/or transcriptomic clock as described herein comprises inducing the age as determined by said epigenetic and/or transcriptomic clock to become “older” (i.e. so that a quantifiable change can be detected) upon introducing one or more ageing-inducing factor into the cell or exposing the cell to one or more exogenous ageing-inducing factor, compared to the cell prior to said introduction of or exposure to the ageing-inducing factor (i.e. the methylation and/or transcriptomic age is older than a non-aged cell). Additionally or alternatively, inducing ageing comprises inducing the age as determined by the epigenetic and/or transcriptomic clock to become older compared to a cell in which the one or more ageing-inducing factor has not been introduced or a cell which has not been exposed to the one or more exogenous ageing-inducing factor.

In further embodiments, “age” is determined using any suitable molecular signature, including those suitable for use as a biological clock. Such biological clocks may therefore be used to determine biological age. Further examples of biological clocks to those mentioned hereinbefore are known in the art and include the measurement of telomere length, the proteomic clock, the metabolomic clock and the ribosomal clock which measures the methylation status of CpG sites within ribosomal DNA (rDNA; Wang & Lemos (2019) Genome Res, doi://www.genome.org/cgi/doi/10.1101/gr.241745.118). It will be readily appreciated that any of these and other molecular signatures/biological clocks may be used independently or in combination (e.g. the epigenetic and transcriptomic clocks may be used together in combination to determine age, or either/both the epigenetic and transcriptomic clocks may be used together with a further biological clock, such as the ribosomal clock). In the context of biological clocks, making a cell “older” will therefore comprise a detectable and quantifiable change in the age determined by these clocks, such as an rDNA methylation (rDNAm) age which is older, upon introducing one or more ageing-inducing factor into the cell or exposing the cell to one or more exogenous ageing-inducing factor, compared to the cell prior to said introduction of or exposure to the ageing-inducing factor. Additionally or alternatively, inducing ageing comprises inducing the age as determined by the biological clock (e.g. the rDNAm age) to become older compared to a cell in which the one or more ageing-inducing factor has not been introduced or a cell which has not been exposed to the one or more exogenous ageing-inducing factor.

The term “inducing ageing” as used herein thus refers to a change in the age of a cell as described herein, in particular making the cell “older”. Such changes in the age of the cell comprise a change of an ageing phenotype, epigenetic clock (i.e. methylation age) and/or transcriptomic clock (i.e. transcriptomic age) as described herein. Thus, in some embodiments inducing ageing in a cell comprises any one or more of the ageing phenotypes described herein, such as inducing one or more of the ageing phenotypes described herein. In further embodiments, inducing ageing in a cell comprises a change in an epigenetic clock and/or a transcriptomic clock, in particular a transcriptomic clock (i.e. the transcriptomic age). It will appreciated that the ageing as described herein is forced upon the cell by introduction of or exposure to the one or more ageing-inducing factor and therefore may be referred to as “artificial”. Thus, in certain embodiments, the ageing induced by the methods of the invention is accelerated compared to the normal ageing of a cell, such as the same type of cell subjected to the methods, that is the ageing of the cell when no artificial ageing has been induced.

The terms “induce ageing”, “inducing ageing”, “induced ageing” and “ageing-inducing” are used herein to indicate the active induction of ageing by the methods of the invention. It will be appreciated that such induction may be temporal or transient, i.e. for a defined period of time, and not permanent. For example, the methods described herein induce ageing in a cell for a period of time (i.e. transiently) while the one or more ageing-inducing factor is introduced into or exogenously exposed to the cell, followed by removal or reduction of the one or more ageing-inducing factor thereby removing the induction of ageing. Thus, in one embodiment ageing is induced in the stem cell or somatic cell for a period of time, followed by removal of the one or more ageing-inducing factor. In a particular embodiment, removing or reducing the one or more ageing-inducing factor comprises active removal or reduction, thereby leading to the reversal of one or more ageing phenotype in the cell. Active removal or reduction of the one or more ageing-inducing factor may include removal of the exogenously provided ageing-inducing factor, inhibition of the activity of the ageing-inducing factor and/or reduction/inactivation of the expression of a sequence encoding the ageing-inducing factor. For example, wherein introducing the one or more ageing-inducing factor comprises one or more expression cassette comprising a sequence encoding the one or more ageing-inducing factor, active removal or reduction may comprise reduction and/or inhibition of the expression of said sequence. As will be readily appreciated by a normal interpretation of the term “active”, such active removal or reduction does not include the natural turnover of an ageing-inducing factor which has been introduced into the cell e.g. by transient transfection. Thus, in one embodiment reduction and/or inhibition of the expression of a sequence encoding the one or more ageing-inducing factor does not comprise passaging the cell following transient transfection of a sequence encoding the one or more ageing-inducing factor. Reduction and/or inhibition of the expression of the sequence encoding the one or more ageing-inducing factor can comprise active regulation (i.e. reduction/inhibition) of said expression by a transcriptional regulator protein which binds to and controls expression at an inducible promoter. Thus, in some embodiments wherein the expression cassette further comprises an inducible promoter which is regulated by a transcriptional regulator protein, expression of the sequence encoding the one or more ageing-inducing factor is reduced and/or inhibited by regulating the activity of the transcriptional regulator protein, in particular actively regulating said activity of the transcriptional regulator protein. For example, wherein the transcriptional regulator protein is a transcriptional activator protein, expression of the sequence encoding the one or more ageing-inducing factors is reduced/inhibited by removal of an exogenous substance (e.g. tetracycline and derivatives thereof or cumate) which activates the transcriptional activator protein from the cell. Alternatively, wherein the transcriptional regulator protein is a transcriptional repressor protein, expression is reduced/inhibited by addition of an exogenous substance (e.g. tetracycline and derivatives thereof or cumate) which deactivates the transcriptional repressor protein. Therefore, in a further embodiment expression of the sequence encoding the ageing-inducing factor or the activity of the transcriptional regulator protein is reduced and/or inactivated by the removal an exogenously supplied substance, such as tetracycline and derivatives thereof or cumate. In a yet further embodiment, introducing one or more ageing-inducing factor into the cell or exposing the cell to one or more exogenous ageing-inducing factor for a period of time comprises introducing one or more expression cassette comprising a sequence encoding said one or more ageing-inducing factor and an inducible promoter which is regulated by a transcriptional regulator protein into the cell, wherein expression of the sequence encoding the ageing-inducing factor or the activity of the transcriptional regulator protein is controlled by an exogenously supplied substance, such as tetracycline and derivatives thereof or cumate.

The period of time for which ageing is induced and the one or more ageing-inducing factor is introduced into the cell/exposed to the cell in step (i) will be appreciated to depend on the ageing-inducing factor and/or the amount of ageing-inducing factor introduced into the cell or to which the cell is exposed. The identity of the stem cell or somatic cell in which ageing is induced may also determine the period of time for which ageing is induced. For example, a proliferating stem or somatic cell may be induced for a shorter period of time than a non- or more slowly proliferating stem or somatic cell. Alternatively, a stem cell with regenerative capacity which reverses an ageing phenotype may be induced for a longer period of time than a somatic cell which does not comprise regenerative capacity. Thus, in some embodiments ageing is induced in step (i) for a period of time for the stem or somatic cell to develop and/or display one or more ageing phenotype as described herein, such as until an ageing phenotype is developed and/or displayed. In further embodiments, ageing is induced in step (i) for a period of time for one or more ageing phenotype as described herein to be measured and/or observed in the stem or somatic cell, such as until an ageing phenotype is measured and/or observed. Thus, in some embodiments an ageing phenotype is induced in step (i) for a period of time. In one embodiment, ageing is induced in step (i) for a period of time of 5 days or more. Thus, in a further embodiment the one or more ageing-inducing factor is introduced into the cell or the cell is exposed to the one or more ageing-inducing factor for 5 days or more. In a yet further embodiment, ageing is induced in step (i) for a period of time of 7 days or less. Thus, in one embodiment the one or more ageing-inducing factor is introduced into the cell or the cell is exposed to the one or more ageing-inducing factor for 7 days or less. In a still further embodiment, ageing is induced in step (i) for a period of time of between 5 and 7 days. Thus, in one embodiment the one or more ageing-inducing factor is introduced into the cell or the cell is exposed to the one or more ageing-inducing factor for between 5 and 7 days. In one embodiment, ageing is induced for a period of time of 5 days. Thus, in one embodiment the one or more ageing-inducing factor is introduced into the cell or the cell is exposed to the one or more ageing-inducing factor for 5 days. In another embodiment, ageing is induced in step (i) for a period of time of 7 days. Thus, in one embodiment the one or more ageing-inducing factor is introduced into the cell or the cell is exposed to the one or more ageing-inducing factor for 7 days. References herein to “ageing is induced for a period of time” also apply to introducing one or more ageing inducing factor or exposing a cell to one or more exogenous ageing-inducing factor for a period of time in step (i). After removal or reduction of the one or more ageing-inducing factor in step (ii), such as active removal or reduction of the ageing-inducing factor as described hereinbefore, it has been observed by the inventors that stem cells (e.g. pluripotent stem cells and iPSCs) reverse the changes in ageing phenotypes seen upon introduction of the ageing-inducing factor and that this reversal is quicker than can be explained by changes in the level of the ageing-inducing factor in the cell (e.g. through protein turnover). Therefore, without being bound by theory it is hypothesised that the unique regenerative capacity of stem cells actively reverses transiently induced ageing phenotypes. As such, in certain embodiments ageing is induced in step (i) in the stem cell or somatic cell for a period of time, followed by removal or reduction of the one or more ageing-inducing factor in step (ii), thereby leading to a reversal of one or more ageing phenotype in the cell. In a particular embodiment, ageing is induced in step (i) in the stem cell or somatic cell for a period of time, followed by active removal or reduction of the one or more ageing-inducing factor in step (ii), thereby leading to reversal of one or more ageing phenotype in the cell. In one embodiment, the one or more ageing phenotype induced in step (i) is the same ageing phenotype reversed upon removal or reduction of the ageing-inducing factor in step (ii). In an alternative embodiment, the one or more ageing phenotype induced in step (i) is a different ageing phenotype to that reversed in step (ii). In another embodiment, one or more of the ageing phenotypes induced in step (i) is the same as one or more of the ageing phenotypes reversed in step (ii).

Thus, in a further aspect of the invention there is provided a method of transiently inducing ageing in a stem cell, said method comprising introducing one or more ageing-inducing factor into the cell or exposing the cell to one or more exogenous ageing-inducing factor for a period of time, followed by removing or reducing the one or more ageing-inducing factor, thereby leading to a reversal of one or more ageing phenotype in the cell. In certain embodiments, removing or reducing the one or more ageing-inducing factor is active removal or reduction as described hereinbefore, leading to reversal of one or more ageing phenotype in the cell.

The methods described herein of transiently ageing a cell (e.g. a stem cell or a somatic cell derived from an iPSC by forward programming) comprising a first step of introducing one or more ageing-inducing factor into the cell or exposing the cell to said ageing-inducing factor, followed by a second step of removing or reducing the ageing-inducing factor, in particular actively removing or reducing the ageing-inducing factor, have not previously been disclosed in the art. For example, US2016/0115444 discloses the ability to accelerate the “maturation” of cells using progerin, and Chojnowski et al. (2020) Aging Cell, 19(3):e13108 (doi: https://doi.org/10.1111%2Facel.13108) discloses the effect on the cell cycle and heterochromatin loss upon progerin expression in cells. However, both US2016/0115444 and Chojnowski et al. (2020) use the transient expression of progerin in cells, and thus neither disclose the reversal of ageing in stem cells or iPSC-derived somatic cells following the removal or reduction of an ageing-inducing factor according to the present invention. In particular, the active removal or reduction of the one or more ageing-inducing factor, thereby leading to the reversal of one or more ageing phenotype in the cell, is not disclosed. Furthermore, Miller et al. (2013) Cell Stem Cell, 13(6):691-705 (doi: https://doi.org/10.1016/j.stem.2013.11.006) discloses the induction of multiple ageing-related markers and characteristics in fibroblasts and neurons following transient/short-term expression of progerin. However, there is no disclosure therein of the removal/reduction of progerin or of a reversal of ageing following the transient expression of progerin, possibly implying that the transient expression of progerin is sufficient to irreversibly induce ageing phenotypes, contrary to the findings of the present inventors as described herein.

Cellular processes and genes involved in the regenerative capacity of stem cells may therefore be screened using the methods described herein, in particular genes and combinations of genes involved in the reversal of an ageing phenotype or the maintenance of a non-aged phenotype, such as that of a stem cell. No such screening methods for the identification of cellular processing or genes involved in the regenerative capacity of stem cells or in the reversal of an ageing phenotype/maintenance of a non-aged phenotype are known in the art.

It will be appreciated that the term “ageing-inducing factor” as used herein may include any condition, agent, compound, gene/gene expression or cellular condition that can induce and/or alter any of the ageing phenotypes described herein, including age as determined using an epigenetic and/or transcriptomic clock or other biological clock/combination of biological clocks. For example, the ageing-inducing factor may be an alternative splice form of Lamin A, such as progerin or a progerin-like truncated form of Lamin A, which alters nuclear morphology, in particular loss of nuclear circularity. Progerin is a nuclear lamina protein associated with Hutchinson-Gilford Progeria Syndrome (HGPS) and is a mutant form of Lamin A lacking 50 amino acids of the C-terminus which prevents removal of a farnesyl group and leads to accumulation of the protein at the nuclear rim/lamina. In another example, the ageing-inducing factor may be a physical stressor. Physical stressors include radiation, such as X-ray radiation, and other inducers of DNA damage which lead to DNA double stand breaks. Thus, in one embodiment the one or more ageing-inducing factor is an alternative splice form of Lamin A. In a particular embodiment, the alternative splice form of Lamin A is progerin. In a further embodiment, the one or more ageing-inducing factor is a progerin-like protein, such as a truncated form of Lamin A which may comprise a deletion of the C-terminal truncation site leading to the improper processing of the protein failure to integrate into the nuclear lamina, causing morphological alterations in the cell which are similar to those caused by progerin. In another embodiment, the one or more ageing-inducing factor is a mitochondrial DNA mutation, such as deletion of DNA polymerase subunit gamma (POLG). In a further embodiment, the one or more ageing-inducing factor is an ageing-inducing compound, such as a telomerase inhibitor (e.g. BIBR1532), reactive oxygen species (ROS) or a compound that disrupts mitochondria function, such as 6-hydroxydopamine (6-OHDA), valinomycin, CCCP, rotenone or hydrogen peroxide. In a yet further embodiment, the ageing-inducing factor is a physical stressor. In a still further embodiment, the ageing-inducing factor is an inducer of DNA damage, such as ionizing radiation (e.g. X-ray radiation), platinum-containing drugs (e.g. cisplatin, oxaliplatin or carboplatin), cyclophosphamide, chlorambucil or temozolomide. In one embodiment, the one or more ageing-inducing factor is radiation, such as X-ray radiation.

In certain embodiments, the ageing phenotype is specific to the cell, such as to the cell type and/or lineage. For example, and without limitation, wherein the cell is a stem cell, such as a pluripotent stem cell, an induced pluripotent stem cell (iPSC), a germline stem cell, a multipotent stem cell, an oligopotent stem cell, a unipotent stem cell or a tissue-specific/tissue-resident stem cell, the ageing phenotype may be reduced proliferation. Alternatively or in addition, wherein the cell is a stem cell, the ageing phenotype may be increased senescence. If the cell is an immune cell, such as a T cell, the ageing phenotype may be increased senescence. If the cell is a cell of the nervous system, such as a neuron or a glia cell, the ageing phenotype may be a phenotype of a neurological degenerative disease, such as reduced process density and/or connectivity, reduced average dendritic length, reduced neurite diameter, downregulation of neuronal marker genes or reduced electrophysiological activity. Thus, in one embodiment wherein the cell is a neuron, the ageing phenotype is a phenotype of a neurological degenerative disease, such as those described herein. If the cell is a muscle cell, such as a myocyte in the heart, the ageing phenotype may be reduced electrophysiological activity or reduced mitochondrial function.

Thus, in one embodiment, the stem cell is a pluripotent stem cell, an induced pluripotent stem cell (iPSC), a germline stem cell, a multipotent stem cell, an oligopotent stem cell, a unipotent stem cell or a tissue-specific/tissue-resident stem cell. Thus, it will be appreciated that cells suitable for methods and uses described herein may include any type of stem cell. For example, the stem cells may be pluripotent stem cells, for example iPSCs, embryonic stem cells or pluripotent stem cells derived by nuclear transfer or cell fusion. It may be preferred that the embryonic stem cell is derived without destruction of the embryo, particularly where the cells are human. In some embodiments, the stem cells are not derived from human or animal embryos, i.e. the invention does not extend to any methods or uses which involve the destruction of human or animal embryos. The stem cells may also include multipotent stem cells, oligopotent stem cells or unipotent stem cells. The stem cells may also include foetal stem cells or adult stem cells, such as hematopoietic stem cells, mesenchymal stem cells, neural stem cells, epithelial stem cells or skin stem cells. In certain aspects, the stem cells may be isolated from umbilical, placenta, amniotic fluid, chorion villi, blastocysts, bone marrow, adipose tissue, brain, peripheral blood, cord blood, menstrual blood, blood vessels, skeletal muscle, skin and liver.

References herein to “pluripotent” refer to cells which have the potential to differentiate into all types/lineages of cell found in an organism. Multipotent stem cells are able to differentiate into a smaller number of cell types than pluripotent cells, such as only those of closely related cell lineages. Oligopotent stem cells can differentiate into only a few cell types, such as lymphoid or myeloid stem cells. Unipotent cells can produce only one cell type and are thus-lineage-specific but have the ability to self-renew which distinguishes them from non-stem cells (e.g. progenitor cells, which cannot self-renew). One form of pluripotent stem cell, known as induced pluripotent stem cells, are of particular interest to the present invention. “Induced pluripotent stem cells” (iPSCs) are cells that have been reprogrammed to an embryonic stem cell-like state by being forced to express genes and factors important for maintaining the defining properties of embryonic stem cells. In 2006, it was shown that overexpression of four specific transcription factors could convert adult cells into pluripotent stem cells. Oct-3/4 and certain members of the Sox gene family have been identified as potentially crucial transcriptional regulators involved in the induction process. Additional genes including certain members of the Klf family, the Myc family, Nanog, and Lin28, may increase the induction efficiency. Examples of the genes which may be used as reprogramming factors to generate iPSCs include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sall4, Esrrb, Tbx3 and Glis1, GATA3, GATA6 and these reprogramming factors may be used singly, or in combination of two or more kinds thereof. In particular, the reprogramming factors may comprise at least the Yamanaka factors, i.e. Oct3/4, Sox2, Klf4 and c-Myc.

In an alternative embodiment, the cell is a somatic cell, such as a somatic cell derived from a reprogrammed iPSC. The iPSC may be as defined herein.

Therefore, according to a further aspect of the invention there is provided a method of transiently inducing ageing in a somatic cell derived from an induced pluripotent stem cell (iPSC) by forward programming, said method comprising:
(i) introducing one or more ageing-inducing factor into the cell or exposing the cell to one or more exogenous ageing-inducing factor for a period of time prior to or after forward programming of the iPSC, and
(ii) removing or reducing the one or more ageing-inducing factor, thereby leading to a reversal of one or more ageing phenotype in the cell.

In a yet further aspect, there is provided a method of transiently inducing ageing in a somatic cell derived from an induced pluripotent stem cell (iPSC) by forward programming, said method comprising introducing one or more ageing-inducing factor into the cell or exposing the cell to one or more exogenous ageing-inducing factor for a period of time prior to or after forward programming of the iPSC, followed by removing or reducing the one or more ageing-inducing factor, thereby leading to a reversal of one or more ageing phenotype in the cell. In certain embodiments, removing or reducing the one or more ageing-inducing factor is active removal or reduction as described hereinbefore, leading to reversal of one or more ageing phenotype in the cell.

In one embodiment, the somatic cell is a neuron (e.g. motor neuron, sensory neuron, GABAergic neuron, glutamatergic neuron or dopaminergic neuron), glia cell, blood cell (e.g. erythrocyte), immune cell (e.g. T cell, B cell, Macrophage, NK cell, neutrophil or granulocyte), liver cell (e.g. hepatocyte, Kupffer cell or stromal cell), muscle cell (e.g. smooth muscle cell), myocyte (e.g. cardiomyocyte), fibroblast, skin cell (e.g. keratinocyte), bone cell, cartilage cell, epithelial cell, endothelial cell or adipocyte. In a further embodiment the cell, such as the somatic cell, is a neuron derived from an iPSC by forward programming.

The term “somatic cell” as used herein includes any type (i.e. lineage) of cell that makes up the body of an organism, excluding germ cells and undifferentiated stem cells. Somatic cells may therefore include, for example and without limitation, neurons, glia cells, blood cells (e.g. leucocytes), liver cells (e.g. hepatocytes), muscle cells (e.g. myocytes) or fibroblasts. In one embodiment, the somatic cell may be an adult cell or a cell derived from an adult which displays one or more detectable characteristics of an adult or non-embryonic cell. References herein to a somatic cell “derived from an iPSC by forward programming” refer to cells which comprise the phenotype and/or characteristics of a somatic cell as defined herein (e.g. the surface phenotype and/or functional characteristics associated with a particular lineage) and have been forward programmed from a pluripotent stem cell which has previously been reprogrammed as described herein, i.e. an iPSC. Forward programming of an iPSC to a somatic cell comprises the introduction of lineage-specific factors, such as transcription factors, or nucleic acids which encode said lineage-specific factors, for example in the form of mRNA or expression cassettes. Alternatively, forward programming may comprise increasing the expression of lineage-specific factors (e.g. lineage-specific transcription factors), such as by increasing the expression of said lineage-specific factor genes and/or their protein expression. The expression of an exogenous or endogenous (in particular an exogenous) transcription factor may be increased. In one embodiment, forward programming comprises introducing into the iPSC a nucleic acid or protein preparation which encodes or provides a lineage-specific transcription factor or combinations thereof, and culturing the cell under conditions suitable for reprogramming the cell into a somatic cell.

It will be understood that increasing the expression of lineage-specific factors, such as transcription factors, in the iPSCs to be forward programmed into somatic cells may include any method known in the art, for example, by induction of expression of one or more expression cassettes previously introduced into the cells, or by introduction of nucleic acids (such as DNA or RNA), polypeptides or small molecules to the cells. Increasing the expression of certain endogenous but transcriptionally repressed genes may also reverse the silencing or inhibitory effect on the expression of these genes by regulating the upstream transcription factor expression or epigenetic modulation. Therefore, methods of the invention may involve culturing the cell population under conditions to artificially increase the expression level of one or more lineage-specific transcription factors.

It will be further understood that such methods of increasing expression, such as by induction of expression of one or more expression cassettes or by introduction of nucleic acids, polypeptides or small molecules as described herein, may also be utilised to introduce one or more ageing-inducing factor into the cell or to expose the cell to one or more exogenous ageing-inducting factor, as well as to alter the expression and/or activity of one or more gene or combination of genes identified as being involved in the reversal of an ageing phenotype or in the maintenance of a non-aged phenotype in a cell by the methods described herein. Thus, references herein to methods of increasing the expression of lineage-specific transcription factors also apply to increasing the expression/introducing the one or more ageing-inducing factor and vice versa, as well as to altering the expression/activity of one or more gene or combination of genes identified by the methods described herein. Related methods of controlling expression may also be used to actively remove/reduce the one or more ageing-inducing factor, by reducing/inhibiting the expression of a sequence encoding said one or more ageing-inducing factor.

Thus, in one embodiment the expression of the lineage-specific transcription factors is increased by contacting the iPSC to be forward programmed with the transcription factors (i.e. the transcription factor proteins). In a further embodiment, the one or more ageing-inducing factor is introduced by contacting the stem cell or somatic cell with said ageing-inducing factor. Delivery may occur using direct electroporation of lineage-specific transcription factor proteins and/or ageing-inducing factor to the cells. Alternatively, delivery of the ageing-inducing factor may be by direct treatment or administration to the cell, such as wherein the ageing-inducing factor is an ageing inducing compound, and/or by exposing cells to physical stressors, such as radiation. As will be understood by the methods described herein, the ageing-inducing factor may be introduced prior to increasing the expression of lineage-specific transcription factors or may be introduced after increasing the expression of lineage-specific transcription factors, i.e. prior to or after forward programming of the iPSC. Thus, in one embodiment one or more ageing-inducing factor is first introduced into the iPSC and is followed by contacting the iPSC with lineage-specific transcription factors. In an alternative embodiment, the iPSC is first contacted with lineage-specific transcription factors and is followed by introducing into the iPSC one or more ageing-inducing factor.

In an alternative embodiment, the expression of the lineage-specific transcription factors is increased by contacting the iPSC to be forward programmed with one or more agents that activate or increase the expression or amount of the transcription factors. In a yet further embodiment, the one or more ageing-inducing factor is introduced by contacting the stem cell and/or somatic cell with one or more agents that activate or increase the expression of said ageing-inducing factor. Expression of the ageing-inducing factor may be activated or increased prior to increasing the expression of lineage-specific transcription factors or may be activated or increased after increasing the expression of lineage-specific transcription factors, i.e. prior to or after forward programming of the iPSC, respectively. Thus, in one embodiment the iPSC is first contacted with one or more agents that activate or increase the expression of one or more ageing-inducing factor and is followed by contacting the iPSC with one or more agents that increase the expression of lineage-specific transcription factors. In an alternative embodiment, the iPSC is first contacted with one or more agents that increase the expression of lineage-specific transcription factors and is followed by contacting the iPSC with one or more agents that activate or increase the expression of one or more ageing-inducing factor.

In one embodiment, the agent is selected from the group consisting of: a nucleic acid (i.e. a polynucleotide, such as a messenger RNA (mRNA) or coding DNA sequence); a protein (e.g. an antibody); an aptamer and small molecule; ribosome; RNAi agent; guide RNA (gRNA); and peptide nucleic acid (PNA) and analogues or variants thereof. In one embodiment, the agent is a transcriptional activation system (e.g. a gRNA for use in a gene activation system such as CRISPR/Cas9 or TALEN) for increasing the expression of the one or more endogenous lineage-specific transcription factors and/or ageing-inducing factor.

### Forward Programming & Introduction/Removal of Ageing-Inducing Factors

The method of forward programming an iPSC to a somatic cell may comprise delivering to the cells a nucleic acid comprising an open reading frame encoding one or more of the lineage-specific transcription factors (e.g. in an expression cassette), the transcription factor protein, or an activator of transcription of the open reading frame encoding the transcription factor. This results in the amount of the lineage-specific transcription factor in the cells being increased, and the cells are programmed to form somatic cells. Said open reading frame may be part of a recombinant expression cassette. Introduction of one or more ageing-inducing factor may also comprise delivering to the cells a nucleic acid comprising an open reading frame encoding said ageing-inducing factor or an activator of transcription of the open reading frame encoding the ageing-inducing factor.

In one embodiment, the nucleic acid comprises a recombinant or exogenous expression cassette comprising the one or more lineage-specific transcription factor sequences (or genes) in a sufficient number to cause forward programming of iPSCs to somatic cells. In a further embodiment, the nucleic acid comprises a recombinant or exogenous expression cassette comprising the one or more ageing-inducing factor sequences (e.g. a sequence encoding the ageing-inducing factor) in a sufficient number to induce ageing of the cell as described herein, such as by inducing an ageing phenotype. The exogenous expression cassette may comprise an externally inducible transcriptional regulatory element for inducible expression of the one or more transcription factors and/or ageing-inducing factor, such as an inducible promoter, e.g. comprising a tetracycline response element or variant thereof. In a yet further embodiment, the nucleic acid comprises a recombinant or exogenous expression cassette comprising the one or more lineage-specific transcription factors and the one or more ageing-inducing factor (e.g. sequences encoding the transcription and ageing-inducing factors), such that forward programming of an iPSC to a somatic cell and ageing of the cell are induced. Thus, the exogenous expression cassette may comprise two or more externally inducible transcriptional regulatory elements for inducible expression of the one or more transcription factors and ageing-inducing factor, such as two or more different inducible transcriptional regulatory elements (e.g. inducible promoters), for inducible expression of the one or more transcription factors and ageing-inducing factor either concurrently or independently.

If expression of the lineage-specific transcription factors and/or one or more ageing-inducing factor is increased by introducing an exogenous sequence encoding the transcription and/or ageing-inducing factor (e.g. the transcription factor gene and/or the ageing-inducing factor gene), then it would be understood that any suitable system for delivering the sequence may be used. The gene delivery system may be a transposon system; a viral gene delivery system; an episomal gene delivery system; or a homologous recombination system such as utilising a zinc finger nuclease, a transcription activator-like effector nuclease (TALENs), or a meganuclease, or a CRISPR/Cas9, or the like.

Alternatively, introduction of a nucleic acid, such as DNA or RNA, into cells may use any suitable methods for nucleic acid delivery for transformation of a cell, as described herein or as would be known to one of ordinary skill in the art. Such methods include, but are not limited to, direct delivery of DNA such as by ex vivo transfection, by injection (including microinjection), by electroporation, by calcium phosphate precipitation, by using DEAE-dextran followed by polyethylene glycol, by direct sonic loading, by liposome mediated transfection, by receptor-mediated transfection, by microprojectile bombardment, by agitation with silicon carbide fibers, by Agrobacterium-mediated transformation, and any combination of such methods. Through the application of these techniques, cells may be stably or transiently transformed. For example, transient transfection of a nucleic acid encoding the one or more ageing-inducing factor may find utility in the present methods for introducing one or more ageing-inducing factor into the cell or exposing the cell to one or more exogenous ageing-inducing factor for a period of time in step (i). Thus, step (i) may comprise transient transfection. In a certain embodiment, wherein the cell is a stem cell, step (i) comprises transient transfection. Furthermore, the one or more expression cassette may be transiently transfected into the cell. Thus, in a further embodiment the one or more expression cassette is transiently transfected into the stem cell. Still further, removing or reducing the one or more ageing-inducing factor in step (ii) may also comprise transient transfection, since such transfection introduces the one or more ageing-inducing factor into the cell for a period of time, after which the nucleic acid encoding the one or more ageing-inducing factor is cleared by, no longer expressed in or expression of which is reduced in the cell. Thus, step (ii) may comprise culturing the cell, i.e. to effectively reduce or remove the one or more ageing-inducing factor in said cell. In one embodiment, step (ii) comprises culturing the stem cell, i.e. to effectively reduce or remove the one or more ageing-inducing factor in said stem cell. Additionally and/or alternatively, step (ii) may comprise passaging the cell in culture. Thus, in a further embodiment step (ii) comprises passaging the stem cell in culture. However, as will be appreciated by the disclosures herein, wherein the introduction/expression of one or more ageing-inducing factor is by controlled expression of a sequence encoding said ageing-inducing factor (e.g. using an exogenously supplied substance), transient transfection of a nucleic acid encoding the one or more ageing-inducing factor is not performed to introduce said ageing-inducing factor for a period of time in step (i). Thus, in certain embodiments step (i) does not comprise transient transfection. In a particular embodiment, wherein the cell is a somatic cell derived from an iPSC by forward programming, step (i) does not comprise transient transfection. In a further embodiment, the one or more expression cassette is not transiently transfected into the cell, i.e. it is stably introduced into the cell as described herein and/or using methods known in the art. In a yet further embodiment, the one or more expression cassette is not transiently transfected into the somatic cell derived from a forward programmed iPSC. According to these embodiments, removing or reducing the one or more ageing-inducing factor in step (ii) also does not comprise transient transfection. For example, removing or reducing the one or more ageing-inducing factor is active removal/reduction which does not rely on the clearing of the nucleic acid by the cell. In one embodiment, step (ii) does not comprise passaging the cell in culture. In another embodiment, step (ii) does not comprise passaging the somatic cell derived from a forward programmed iPSC in culture.

Further, the expression cassette (e.g. an inducible recombinant expression cassette comprising sequences encoding lineage-specific transcription factors and/or one or more ageing-inducing factor) may include cleavable sequences. Such sequences are sequences that are recognised by an entity capable of specifically cutting DNA, and include restriction sites, which are the target sequences for restriction enzymes or sequences for recognition by other DNA cleaving entities, such as nucleases, recombinases, ribozymes or artificial constructs. At least one cleavable sequence may be included, but preferably two or more are present. These cleavable sequences may be at any suitable point in the cassette, such that a selected portion of the cassette, or the entire cassette, can be selectively removed if desired. The cleavable sites may thus flank the part/all of the genetic sequence that it may be desired to remove. The method may therefore also comprise removal of the expression cassette and/or the genetic material.

### Vectors

In one embodiment, the lineage-specific transcription factors and/or one or more ageing-inducing factor are introduced into the cell population using a vector. One of skill in the art would be well equipped to construct a vector through standard recombinant techniques. Vectors include but are not limited to plasmids, cosmids, viruses (bacteriophage, animal viruses and plant viruses) and artificial chromosomes (e.g. YACs).

In one embodiment, the vector is a viral vector. The viral gene delivery system may be an RNA-based or DNA-based viral vector. Viral vectors include retroviral vectors, lentiviral vectors (e.g. derived from HIV-1, HIV-2, SIV, BIV, FIV etc.), gammaretroviral vectors, adenoviral (Ad) vectors (including replication competent, replication deficient and gutless forms thereof), adeno-associated virus-derived (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumour virus vectors, Rous sarcoma virus vectors and Sendai virus vectors. In a further embodiment, the viral vector is selected from: a lentiviral vector, an adeno-associated virus vector or a Sendai virus vector. In a yet further embodiment, the viral vector is a lentiviral vector.

Lentiviral vectors are well known in the art. Lentiviral vectors are complex retroviruses capable of integrating randomly into the host cell genome, which, in addition to the common retroviral genes gag, pol, and env, contain other genes with regulatory or structural function (e.g. accessory genes Vif, Nef, Vpu, Vpr). Lentiviral vectors have the advantage of being able to infect non-dividing cells and can be used for both in vivo and ex vivo gene transfer and expression of nucleic acid sequences. For example, recombinant lentiviral vector capable of infecting a non-dividing cell wherein a suitable host cell is transfected with two or more vectors carrying the packaging functions, namely gag, pol and env, as well as rev and tat.

In one embodiment, a nucleic acid sequence encoding the lineage-specific transcription factors and/or one or more ageing-inducing factor is introduced into a cell by a plasmid. In one embodiment, at least one nucleic acid sequence encoding a lineage-specific transcription factor is introduced into a cell on a single plasmid. In a further embodiment, at least one nucleic acid sequence encoding the lineage-specific transcription factors and the one or more ageing-inducing factor is introduced into the cell on a single plasmid. In an alternative embodiment, the sequences encoding the lineage-specific transcription factors and the one or more ageing-inducing factor are introduced into the cell on separate plasmids.

In one embodiment, the plasmid is episomal. Episomal vectors are able to introduce large fragments of DNA into a cell but are maintained extra-chromosomally, replicated once per cell cycle, partitioned to daughter cells efficiently, and elicit substantially no immune response. In alternative embodiments, an Epstein-Barr virus (EBV)-based episomal vector, a yeast-based vector, an adenovirus-based vector, a simian virus 40 (SV40)-based episomal vector, or a bovine papilloma virus (BPV)-based vector may be used.

### Site-Specific Delivery and Targeting

Any suitable technique for insertion of a nucleic acid sequence into a specific sequence may be used, and several are described in the art. Suitable techniques include any method which introduces a break at the desired location and permits recombination of the vector into the gap. Thus, a crucial first step for targeted site-specific genomic modification is the creation of a double-strand DNA break (DSB) at the genomic locus to be modified. Distinct cellular repair mechanisms can be exploited to repair the DSB and to introduce the desired sequence, and these are non-homologous end joining repair (NHEJ), which is more prone to error; and homologous recombination repair (HR) mediated by a donor DNA template, that can be used to insert inducible cassettes.

Several techniques exist to allow customised site-specific generation of DSB in the genome. Many of these involve the use of customized endonucleases, such as zinc finger nucleases, TALENs or the clustered regularly interspaced short palindromic repeats/CRISPR associated protein (CRISPR/Cas9) system.

Zinc finger nucleases are artificial enzymes which are generated by fusion of a zinc-finger DNA-binding domain to the nuclease domain of the restriction enzyme Fokl. The latter has a non-specific cleavage domain which must dimerise in order to cleave DNA. This means that two zinc finger nuclease monomers are required to allow dimerisation of the Fokl domains and to cleave the DNA. The DNA binding domain may be designed to target any genomic sequence of interest, and is a tandem array of Cys2His2 zinc fingers, each of which recognises three contiguous nucleotides in the target sequence. The two binding sites are separated by 5-7 bp to allow optimal dimerization of the Fokl domains. The enzyme thus is able to cleave DNA at a specific site, and target specificity is increased by ensuring that two proximal DNA-binding events must occur to achieve a double-strand break.

Transcription activator-like effector nucleases, or TALENs, are dimeric transcription factor/nucleases. They are made by fusing a TAL effector DNA-binding domain to a DNA cleavage domain (a nuclease). Transcription activator-like effectors (TALEs) can be engineered to bind practically any desired DNA sequence, so when combined with a nuclease, DNA can be cut at specific locations. TAL effectors are proteins that are secreted by Xanthomonas bacteria, the DNA binding domain of which contains a repeated highly conserved 33-34 amino acid sequence with divergent 12th and 13th amino acids. These two positions are highly variable and show a strong correlation with specific nucleotide recognition. This straightforward relationship between amino acid sequence and DNA recognition has allowed for the engineering of specific DNA-binding domains by selecting a combination of repeat segments containing appropriate residues at the two variable positions. TALENs are thus built from arrays of 33 to 35 amino acid modules, each of which targets a single nucleotide. By selecting the array of the modules, almost any sequence may be targeted. Again, the nuclease used may be Fokl or a derivative thereof.

Three types of CRISPR mechanisms have been identified, of which type II is the most studied. The CRISPR/Cas9 system (type II) utilises the Cas9 nuclease to make a double-stranded break in DNA at a site determined by a short guide RNA. The CRISPR/Cas system is a prokaryotic immune system that confers resistance to foreign genetic elements. CRISPR are segments of prokaryotic DNA containing short repetitions of base sequences. Each repetition is followed by short segments of “protospacer DNA” from previous exposures to foreign genetic elements. CRISPR spacers recognise and cut the exogenous genetic elements using RNA interference. The CRISPR immune response occurs through two steps: CRISPR-RNA (crRNA) biogenesis and crRNA-guided interference. CrRNA molecules are composed of a variable sequence transcribed from the protospacer DNA and a CRISPR repeat. Each crRNA molecule then hybridizes with a second RNA, known as the trans-activating CRISPR RNA (tracrRNA) and together these two eventually form a complex with the nuclease Cas9. The protospacer DNA encoded section of the crRNA directs Cas9 to cleave complementary target DNA sequences, if they are adjacent to short sequences known as protospacer adjacent motifs (PAMs). This natural system has been engineered and exploited to introduce DSB breaks in specific sites in genomic DNA, amongst many other applications. In particular, the CRISPR type II system from Streptococcus pyogenes may be used. At its simplest, the CRISPR/Cas9 system comprises two components that are delivered to the cell to provide genome editing: the Cas9 nuclease itself and a guide RNA (gRNA). The gRNA is a fusion of a customised, site-specific crRNA (directed to the target sequence) and a standardised tracrRNA.

Once a DSB has been made, a donor template with homology to the targeted locus is supplied; the DSB may be repaired by the homology-directed repair (HDR) pathway allowing for precise insertions to be made.

Derivatives of this system are also possible. Mutant forms of Cas9 are available, such as Cas9D10A with only nickase activity. This means it cleaves only one DNA strand and does not activate NHEJ. Instead, when provided with a homologous repair template, DNA repairs are conducted via the high-fidelity HDR pathway only. Cas9D10A may be used in paired Cas9 complexes designed to generate adjacent DNA nicks in conjunction with two sgRNAs complementary to the adjacent area on opposite strands of the target site, which may be particularly advantageous.

The elements for making the double-strand DNA break may be introduced in one or more vectors, such as plasmids, for expression in the stem or somatic cell described herein.

Thus, any method of making specific, targeted double strand breaks in the genome in order to affect the insertion of a gene/inducible cassette may be used in the method of the invention. It may be preferred that the method for inserting the gene/inducible cassette utilises any one or more of zinc finger nucleases, TALENs and/or CRISPR/Cas9 systems or any derivative thereof.

Once the DSB has been made by any appropriate means, the gene/inducible cassette for insertion may be supplied in any suitable fashion as described herein. The gene/inducible cassette and associated genetic material form the donor DNA for repair of the DNA at the DSB and are inserted using standard cellular repair machinery/pathways. How the break is initiated will alter which pathway is used to repair the damage, as noted above.

### Controlled Expression

In one embodiment, expression of the lineage-specific transcription factors is under controlled transcription. In this aspect of the invention, the transcription and translation (expression) of the transcription factors may be controlled within the cell. This permits overexpression of the transcription factors, if required. In a further embodiment, expression of the one or more ageing-inducing factor is under controlled transcription. Thus, in a certain embodiment the one or more ageing-inducing factor is introduced into the cell using one or more expression cassette comprising a sequence encoding the one or more ageing-inducing factor, i.e. introducing one or more ageing-inducing factor in step (i) comprises one or more expression cassette comprising a sequence encoding the one or more ageing-inducing factor.

An exogenous expression cassette carrying the lineage-specific transcription factors and/or one or more ageing-inducing factor may comprise an externally inducible transcriptional regulatory element (i.e. an inducible promoter) for inducible expression of the transcription and/or ageing-inducing factors. Said inducible expression cassette may be controlled by addition of an exogenous substance. Whatever culturing conditions are used, the exogenous substance will control expression of the genetic sequence within the inducible expression cassette; and may either be supplied continuously and then withdrawn in order to induce transcription or supplied as transcription is required, dependent upon its mode of action.

Expression of the lineage-specific transcription factors and/or one or more ageing-inducing factor described herein may be increased using the dual cassette expression system described in WO2018096343, which is incorporated herein by reference. This system targets genetic safe harbour (GSH) sites which provides a reduced risk of epigenetic silencing of the inserted genetic material.

Therefore, in one embodiment a sequence encoding lineage-specific transcription factors and/or one or more ageing-inducing factors as described herein are introduced into the stem or somatic cell using a method comprising:
targeted insertion of a gene encoding a transcriptional regulator protein into a first genetic safe harbour site of the cell; and
targeted insertion of an inducible cassette into a second genetic safe harbour site of the cell, wherein said inducible cassette comprises said transcription factor and/or one or more ageing-inducing factor sequences operably linked to an inducible promoter, and said promoter is regulated by the transcriptional regulator protein.

These embodiments of the invention provide a dual expression cassette system. The insertion of the gene encoding a transcriptional regulator protein into the first genetic safe harbour (GSH) provides the control mechanism for the expression of the inducible cassette which is operably linked to the inducible promoter and inserted into a second GSH site. In one embodiment, the first and second GSH are different.

In a particular embodiment, a gene encoding a transcriptional regulator protein is inserted into a first genetic safe harbour site and the expression cassette comprising a sequence encoding the one or more ageing-inducing factor is inserted into a second genetic safe harbour site,
wherein the expression cassette further comprises an inducible promoter which is regulated by the transcriptional regulator protein, and
wherein the first and second genetic safe harbour sites are different.

In an alternative embodiment, the expression cassette and a gene encoding a transcriptional regulator protein are inserted into a genetic safe harbour site, such as a single genetic safe harbour site,
wherein the expression cassette further comprises an inducible promoter which is regulated by the transcriptional regulator protein.

A GSH site is a locus within the genome wherein a gene or other genetic material may be inserted without any deleterious effects on the cell or on the inserted genetic material. Most beneficial is a GSH site in which expression of the inserted gene sequence is not perturbed by any read-through expression from neighbouring genes and expression of the inducible cassette minimises interference with the endogenous transcription programme. More formal criteria have been proposed that assist in the determination of whether a particular locus is a GSH site in future (Papapetrou et al. (2011)). These criteria include a site that is: (i) 50 kb or more from the 5′ end of any gene; (ii) 300 kb or more from any gene related to cancer; (iii) 300 kb or more from any microRNA (miRNA); (iv) located outside a transcription unit; and (v) located 1 outside ultraconserved regions (UCR). It may not be necessary to satisfy all of these proposed criteria, since GSH already identified do not fulfil all of the criteria. It is thought that a suitable GSH will satisfy at least 2, 3, 4 or all of these criteria. Any suitable GSH site may be used in the method of the invention, on the basis that the site allows insertion of genetic material without deleterious effects to the cell and permits transcription of the inserted genetic material. Those skilled in the art may use these simplified criteria to identify a suitable GSH, and/or the more formal criteria set out above.

In one embodiment, the first and second genetic safe harbour sites (GSHs) are selected from (in particular any two) of the hROSA26 locus, the AAVS1 locus, the CLYBL gene, the CCR5 gene or the HPRT gene. Insertions specifically within genetic safe harbour sites is preferred over random genome integration, since this is expected to be a safer modification of the genome, and is less likely to lead to unwanted side effects such as silencing natural gene expression or causing mutations that lead to cancerous cell types. In another embodiment, the single genetic safe harbour site is selected from any GSH described herein.

The adeno-associated virus integration site 1 locus (AAVS1) is located within the protein phosphatase 1, regulatory subunit 12C (PPP1R12C) gene on human chromosome 19, which is expressed uniformly and ubiquitously in human tissues. AAVS1 has been shown to be a favourable environment for transcription, since it comprises an open chromatin structure and native chromosomal insulators that enable resistance of the inducible cassettes against silencing. There are no known adverse effects on the cell resulting from disruption of the PPP1R12C gene. Moreover, an inducible cassette inserted into this site remains transcriptionally active in many diverse cell types.

The hROSA26 site has been identified on the basis of sequence analogy with a GSH from mice (ROSA26—reverse oriented splice acceptor site #26). The hROSA26 locus is on chromosome 3 (3p25.3) and can be found within the Ensembl database (GenBank: CR624523). The integration site lies within the open reading frame (ORF) of the THUMPD3 long non-coding RNA (reverse strand). Since the hROSA26 site has an endogenous promoter, the inserted genetic material may take advantage of that endogenous promoter, or alternatively may be inserted operably linked to a promoter.

Intron 2 of the Citrate Lyase Beta-like (CLYBL) gene, on the long arm of Chromosome 13, was identified as a suitable GSH since it is one of the identified integration hot-spots of the phage derived phiC31 integrase. Studies have demonstrated that randomly inserted inducible cassettes into this locus are stable and expressed. It has been shown that insertion of inducible cassettes at this GSH do not perturb local gene expression (Cerbini et al. (2015)). CLYBL thus provides a GSH which may be suitable for use in the present invention.

CCR5, which is located on chromosome 3 (position 3p21.31) is a gene which codes for HIV-1 major co-receptor. Interest in the use of this site as a GSH arises from the null mutation in this gene that appears to have no adverse effects, but predisposes to HIV-1 infection resistance. Zinc-finger nucleases that target the third exon have been developed, thus allowing for insertion of genetic material at this locus.

The hypoxanthine-guanine phosphoribosyltransferase (HPRT) gene encodes a transferase enzyme that plays a central role in the generation of purine nucleotides through the purine salvage pathway.

GSH in other organisms have been identified and include ROSA26, HRPT and Hipp11 (H11) loci in mice. Mammalian genomes may include GSH sites based upon pseudo attP sites. For such sites, hiC31 integrase, the Streptomyces phage-derived recombinase, has been developed as a non-viral insertion tool, because it has the ability to integrate an inducible cassette-containing plasmid carrying an attB site into pseudo attP sites.

Technically, the insertions into the first and/or second GSH may occur on one chromosome, or on both chromosomes. The GSH exists at the same genetic loci on both chromosomes of diploid organisms. Insertion within both chromosomes is advantageous since it may enable an increase in the level of transcription from the inserted genetic material within the inducible cassette, thus achieving particularly high levels of transcription.

Specific insertion of genetic material into the particular GSH based upon customised site-specific generation of DNA double-strand breaks at the GSH may be achieved. The genetic material may then be introduced using any suitable mechanism, such as homologous recombination. Any method of making a specific DSB in the genome may be used, but preferred systems include CRISPR/Cas9 and modified versions thereof, zinc finger nucleases and the TALEN system.

One or more genetic sequences may be controllably transcribed from within the second and/or further GSH or from within the single GSH. Indeed, the inducible cassette may contain 1, 2, 3, 4, 5, 6, 7, 8, 9 or 10 genetic sequences (e.g. ageing-inducing factor sequences) which it is desired to insert into the GSH and the transcription of which be controllably induced. Therefore, the lineage-specific transcription factors and/or the one or more ageing-inducing factors may be included within the same cassette introduced into the second genetic safe harbour site. For example, the lineage-specific transcription factors and/or one or more ageing-inducing factors may be included in several mono-cistronic constructs or one or more bi-cistronic or tri-cistronic construct as required. It will be understood that similar combinations of constructs may be used to achieve higher orders of expression. In one embodiment, the lineage-specific transcription factors and the one or more ageing-inducing factor are included in one bi-cistronic construct.

Alternatively, in instances where sequences encoding lineage-specific transcription factors and one or more ageing-inducing factors are used, the sequence(s) encoding lineage-specific transcription factors may be introduced into separate GSHs and/or under the control of different inducible promoters to the sequence(s) encoding the one or more ageing-inducing factor. Therefore, in one embodiment the lineage-specific transcription factors are introduced into a separate GSH to the ageing-inducing factor. This may be achieved by utilising two or more different GSH sites or by utilising the fact that a GSH exists at the same genetic loci on both chromosomes of diploid organisms, e.g. introducing the transcription factors into the GSH on one chromosome and the one or more ageing-inducing factor into the same GSH on the other chromosome. This embodiment is advantageous if different expression levels or timing of expression of the transcription factors and ageing-inducing factor is desired. In an alternative embodiment, the sequences encoding the lineage-specific transcription factors and one or more ageing-inducing factor are introduced into the same GSH but are under the control of different inducible promoters. The use of different inducible promoters also achieves different expression levels or timing of expression of the transcription factors and ageing-inducing factor. For example, use of different inducible promoters allows the introduction of an ageing-inducing factor into a somatic cell derived from a forward programmed iPSC either prior to said forward programming (i.e. wherein expression of the ageing-inducing factor has been induced before expression of the lineage-specific transcription factors) or after said forward programming (i.e. wherein expression of the ageing-inducing factor is induced after expression of the lineage-specific transcription factors). Thus, in certain embodiments wherein the cell is a somatic cell derived from an iPSC by forward programming, the sequence encoding the one or more ageing-inducing factor is introduced into the same GSH as the sequence encoding the lineage-specific transcription factors, wherein each of the ageing-inducing factor encoding sequences and lineage-specific transcription factor sequences are under the control of different inducible promoters.

A transcriptional regulator protein is a protein that binds to DNA, preferably sequence-specifically to a DNA site located in or near a promoter, and either facilitating the binding of the transcription machinery to the promoter, and thus transcription of the DNA sequence (a transcriptional activator) or blocks this process (a transcriptional repressor).

The DNA sequence that a transcriptional regulator protein binds to is called a transcription factor-binding site or response element, and these are found in or near the promoter of the regulated DNA sequence. Transcriptional activator proteins bind to the response element and promote gene expression. Such proteins are preferred in the methods of the present invention for controlling inducible cassette expression. Transcriptional repressor proteins bind to the response element and prevent gene expression.

Transcriptional regulator proteins may be activated or deactivated by a number of mechanisms including binding of a substance, interaction with other transcription factors (e.g. homo- or hetero-dimerisation) or coregulatory proteins, phosphorylation, and/or methylation. The transcriptional regulator protein may be controlled by activation or deactivation.

If the transcriptional regulator protein is a transcriptional activator protein, it is preferred that the transcriptional activator protein requires activation. This activation may be through any suitable means, but it is preferred that the transcriptional regulator protein is activated through the addition of an exogenous substance to the cell. The supply of an exogenous substance to the cell can be controlled, and thus the activation of the transcriptional regulator protein can be controlled. Alternatively, an exogenous substance can be supplied in order to deactivate a transcriptional regulator protein, and then supply withdrawn in order to activate the transcriptional regulator protein.

If the transcriptional regulator protein is a transcriptional repressor protein, it is preferred that the transcriptional repressor protein requires deactivation. Thus, a substance is supplied to prevent the transcriptional repressor protein repressing transcription, and thus transcription is permitted.

Any suitable transcriptional regulator protein may be used, preferably one that may be activated or deactivated. It is preferred that an exogenous substance may be supplied to control the transcriptional regulator protein. Such transcriptional regulator proteins are also called inducible transcriptional regulator proteins.

Tetracycline-Controlled Transcriptional Activation is a method of inducible gene expression where transcription is reversibly turned on or off in the presence of the antibiotic tetracycline or one of its derivatives (e.g. doxycycline which is more stable). In this system, the transcriptional activator protein is tetracycline-responsive transcriptional activator protein (rtTa) or a derivative thereof. The rtTA protein is able to bind to DNA at specific TetO operator sequences. Several repeats of such TetO sequences are placed upstream of a minimal promoter (such as the CMV promoter), which together form a tetracycline response element (TRE). There are two forms of this system, depending on whether the addition of tetracycline or a derivative activates (Tet-On) or deactivates (Tet-Off) the rtTA protein.

In a Tet-Off system, tetracycline or a derivative thereof binds rtTA and deactivates the rtTA, rendering it incapable of binding to TRE sequences, thereby preventing transcription of TRE-controlled genes. This system was first described in Gossen et al. (1992).

The Tet-On system is composed of two components; (1) the constitutively expressed tetracycline-responsive transcriptional activator protein (rtTa) and the rtTa-sensitive inducible promoter (Tet Responsive Element, TRE). This may be bound by tetracycline or its more stable derivatives, including doxycycline (dox), resulting in activation of rtTa, allowing it to bind to TRE sequences and inducing expression of TRE-controlled genes. The use of this may be preferred in the method of the invention.

Thus, the transcriptional regulator protein may be a tetracycline-responsive transcriptional activator (rtTa) protein, which can be activated or deactivated by tetracycline or one of its derivatives, which are supplied exogenously. If the transcriptional regulator protein is rtTA, then the inducible promoter inserted into the second GSH site includes the tetracycline response element (TRE). The exogenously supplied substance is tetracycline or one of its derivatives. Variants and modified rtTa proteins may also be used in the methods of the invention, these include Tet-On Advanced transactivator (also known as rtTA2S-M2) and Tet-On 3G (also known as rtTA-V16, derived from rtTA2S-S2).

The tetracycline response element (TRE) generally consists of 7 repeats of the 19 bp bacterial TetO sequence separated by spacer sequences, together with a minimal promoter. Variants and modifications of the TRE sequence are possible, since the minimal promoter can be any suitable promoter. Preferably the minimal promoter shows no or minimal expression levels in the absence of rtTa binding. The inducible promoter inserted into the second GSH may thus comprise a TRE.

A modified system based upon tetracycline control is the T-REX System (Thermo-Fisher Scientific), in which the transcriptional regulator protein is a transcriptional repressor protein, TetR. The components of this system include an inducible promoter comprising a strong human cytomegalovirus immediate-early (CMV) promoter and two tetracycline operator 2 (TetO2) sites, and a Tet repressor (TetR). In the absence of tetracycline, the Tet repressor forms a homodimer that binds with extremely high affinity to each TetO2 sequence in the inducible promoter, and prevents transcription from the promoter. Once added, tetracycline binds with high affinity to each Tet repressor homodimer rendering it unable to bind to the Tet operator. The Tet repressor: tetracycline complex then dissociates from the Tet operator and allows induction of expression. In this instance, the transcriptional regulator protein is TetR and the inducible promoter comprises two TetO2 sites. The exogenously supplied substance is tetracycline or a derivative thereof.

The cumate switch is another method of inducible gene expression where transcription is reversibly turned on or off in the presence of the cumate. This system is available in both activator and repressor configurations, where the presence of cumate leads to the repression of transcription or activation of transcription, respectively. In the repressor configuration, regulation is mediated by the binding of the repressor (CymR) to the operator site (CuO), placed downstream of a constitutive promoter. Addition of cumate, a small molecule, relieves the repression and allows transcription to proceed. In the activator configuration, a chimeric transactivator (cTA) protein, formed by the fusion of CymR with the activation domain of VP16, is able to activate transcription when bound to the CuO operator site, placed upstream of the constitutive promoter. Cumate addition abrogates DNA binding and therefore transactivation by cTA, stopping transcription.

Thus, in some embodiments the transcriptional regulator protein may be a Tet-responsive transcriptional activator protein (rtTa) and/or a cumate repressor (CymR). In one embodiment, expression of the lineage-specific transcription factors is under the control of a Tet-responsive element. Thus in a further embodiment, the sequence encoding the lineage-specific transcription factors comprises a Tet-response element (TRE). In a yet further embodiment, expression of the sequence encoding the lineage-specific transcription factors is controlled, such as induced, by exogenous addition of tetracycline or a derivative thereof (e.g. doxycycline). In another embodiment, expression of the one or more ageing-inducing factor is under the control of a cumate switch. Thus, in a yet further embodiment, the sequence encoding the one or more ageing-inducing factors comprises a CuO site. In a still further embodiment, expression of the sequence encoding the ageing-inducing factor or the activity of the transcriptional regulator protein is controlled by an exogenously supplied substance, such as tetracycline and derivatives thereof or cumate. In a particular embodiment, expression of the ageing-inducing factor is controlled, such as induced, by exogenous addition of cumate.

Therefore, in other embodiments wherein the cell is a somatic cell derived from an iPSC by forward programming, said forward programming of the iPSC to a somatic cell is by expression of lineage-specific transcription factors upon exogenous addition of tetracycline or a derivative thereof (e.g. doxycycline) and ageing is induced by expression of the ageing-inducing factor upon exogenous addition of cumate. In one embodiment, forward programming of the iPSC to a somatic cell by expression of lineage-specific transcription factors upon exogenous addition of tetracycline or a derivative thereof is followed by inducing ageing by expression of the ageing-inducing factor upon exogenous addition of cumate. In an alternative embodiment, ageing is induced by expression of the ageing-inducing factor upon exogenous addition of cumate followed by forward programming of the iPSC to a somatic cell by expression of lineage-specific transcription factors upon exogenous addition of tetracycline or a derivative thereof.

Other inducible expression systems are known and can be used in the method of the invention. These include the Complete Control Inducible system from Agilent Technologies. This is based upon the insect hormone ecdysone or its analogue ponasterone A (ponA) which can activate transcription in mammalian cells which are transfected with both the gene for the Drosophila melanogaster ecdysone receptor (EcR) and an inducible promoter comprising a binding site for the ecdysone receptor. The EcR is a member of the retinoid-X-receptor (RXR) family of nuclear receptors. In humans, EcR forms a heterodimer with RXR that binds to the ecdysone-responsive element (EcRE). In the absence of PonA, transcription is repressed by the heterodimer.

Thus, the transcriptional regulator protein can be a repressor protein, such as CymR, an ecdysone receptor or a derivative thereof. Examples of the latter include the VgEcR synthetic receptor from Agilent technologies which is a fusion of EcR, the DNA binding domain of the glucocorticoid receptor and the transcriptional activation domain of Herpes Simplex Virus VP16. The inducible promoter comprises the EcRE sequence or modified versions thereof together with a minimal promoter. Modified versions include the E/GRE recognition sequence of Agilent Technologies, in which mutations to the sequence have been made. The E/GRE recognition sequence comprises inverted half-site recognition elements for the retinoid-X-receptor (RXR) and GR binding domains. In all permutations, the exogenously supplied substance is ponasterone A, which removes the repressive effect of EcR or derivatives thereof on the inducible promoter, and allows transcription to take place.

In one embodiment, the stem cell or somatic cell is from a mammal. In a further embodiment, the mammal is a human. Thus, in a particular embodiment the stem cell or somatic cell is from a human and is a human stem cell or a human somatic cell, such as a somatic cell derived from a re-programmed human iPSC. In an alternative embodiment, the mammal is a mouse, optionally such that the stem cell or somatic cell is a mouse stem cell or a mouse somatic cell, such as a somatic cell derived from a re-programmed mouse iPSC.

In other aspects of the invention there is provided an artificially aged cell produced by the methods described herein. In one aspect, there is provided an artificially aged stem cell produced by the methods described herein. Thus, in one embodiment there is provided an artificially aged stem cell. In another aspect, there is provided an artificially aged somatic cell derived from an iPSC by forward programming produced by the methods described herein. Thus, in a further embodiment there is provided an artificially aged somatic cell.

### Uses and Screening Methods

In further aspects of the invention, there is provided a use of an artificially aged cell described herein in a method of identifying a gene or a combination of genes involved in the reversal of an ageing phenotype or in the maintenance of a non-aged phenotype in the cell. In one aspect, there is provided the use of an artificially aged stem cell as described herein in a method of identifying a gene or a combination of genes involved in the reversal of an ageing phenotype or in the maintenance of a non-aged phenotype in the cell. In another aspect, there is provided the use of an artificially aged somatic cell derived from an iPSC by forward programming as described herein in a method of identifying a gene or a combination of genes involved in the reversal of an ageing phenotype or in the maintenance of a non-aged phenotype in the cell. In one embodiment, the use comprises use of an artificially aged stem cell and an artificially aged somatic cell derived from an iPSC by forward programming as described herein.

Such uses are based on the observation described herein that after removal or reduction of one or more ageing-inducing factor stem cells reverse the changes in ageing phenotypes seen upon the induction of ageing. Therefore, the unique regenerative capacity of stem cells which actively reverses the induced ageing phenotypes may be used to screen genes and combinations of genes involved in the reversal of an ageing phenotype or the maintenance of a non-aged phenotype. In other words, by selectively targeting and inactivating, knocking-out or activating a gene suspected to be involved in the reversal of an ageing phenotype or in the maintenance of a non-aged phenotype in a stem cell, the effect of said targeting on the induction of ageing by the methods herein may be observed.

Identification of genes and combinations of genes may be by sequencing. For example, genes or combinations of genes involved in reversal of an ageing phenotype or maintenance of a non-aged phenotype which have been targeted may be identified by transcriptome sequencing to determine the expression, such as the expression level, following targeting. Alternatively, said genes or combinations of genes may be identified by detecting the agent used to target said gene(s). In one embodiment, identification of genes or combinations of genes involved in reversal of an ageing phenotype or maintenance of a non-aged phenotype is by detection of the guide RNA used to target said gene or detection of the multiple guide RNAs used to target said combination of genes. In a further embodiment, the detection of the guide RNA is by sequencing. In a yet further embodiment, the guide RNA comprises a barcode sequence and detection of the guide RNA is by sequencing using said barcode sequence.

Thus, according to one aspect of the invention there is provided a method of identifying a gene or a combination of genes involved in the reversal of an ageing phenotype or in the maintenance of a non-aged phenotype in a cell, said method comprising the steps of:
i) introducing one or more ageing-inducing factor into the cell or exposing the cell to one or more exogenous ageing-inducing factor for a period of time;
ii) optionally performing a loss-of-function, inhibitory, knock-out, gain-of-function or combinatorial screen in the cell;
iii) measuring an ageing phenotype of the cell; and
iv) identifying a gene or a combination of genes as being involved in the reversal of an ageing phenotype or in the maintenance of a non-aged phenotype when the ageing phenotype is altered and/or its progression is altered in the cell when a loss-of-function, inhibitory, knock-out, gain-of-function or combinatorial screen is performed compared to when the cell is not subjected to a screen.

In one embodiment, the method of identifying a gene or a combination of genes involved in the reversal of an ageing phenotype or in the maintenance of a non-aged phenotype in the cell using the artificially aged stem cell and/or somatic cell derived from an iPSC by forward programming described herein comprises the steps of:
i) performing a loss-of-function, inhibitory, knock-out, gain-of-function or combinatorial screen in the artificially aged stem cell or somatic cell;
ii) measuring an ageing phenotype of the cell; and
iii) identifying a gene or a combination of genes as being involved in the reversal of an ageing phenotype or in the maintenance of a non-aged phenotype when the ageing phenotype is altered and/or its progression is altered in the cell compared to an artificially aged stem cell or somatic cell as defined herein which is not subjected to a loss-of-function, inhibitory, knock-out, gain-of-function or combinatorial screen.

In certain embodiments, the ageing phenotype is as described herein. In one embodiment, the ageing phenotype measured in the method is the transcriptomic clock of the cell, optionally in combination with one or more further biological clock as described herein (e.g. the epigenetic clock and/or the ribosome clock). In a further embodiment, the gene or combination of genes involved in the reversal of an ageing phenotype or in the maintenance of a non-aged phenotype is identified as described herein. In a particular embodiment, the gene or combination of genes is identified by sequencing, such as described herein.

In some embodiments, the somatic cell is a neuron, glia cell, blood cell, immune cell, liver cell, hepatocyte, muscle cell, myocyte, fibroblast, skin cell, bone cell, cartilage cell, epithelial cell, endothelial cell or adipocyte. In further embodiments, the somatic cell is derived from an iPSC by forward programming as described herein. In a particular embodiment, the somatic cell is a neuron derived from an iPSC by forward programming. In a further embodiment, wherein the somatic cell is a neuron as described herein, the ageing phenotype may optionally be a phenotype of a neurological degenerative disease, such as reduced process density and/or connectivity, reduced average dendritic length, reduced neurite diameter, downregulation of neuronal marker genes or reduced electrophysiological activity

In one embodiment, the loss-of-function, inhibitory, knock-out, gain-of-function or combinatorial screen comprises nucleic acids, polypeptides (e.g. proteins, such as antibodies), aptamers and/or small molecules (e.g. small molecule compounds). Such nucleic acids, polypeptides, aptamers and small molecules include those which act as activators/agonists as well as those which act as inhibitors/antagonists. Thus, it will be appreciated that the methods described herein find utility in screening any modulator (e.g. modulators of expression or activity) of a gene or a combination of genes involved in the reversal of an ageing phenotype or in the maintenance of a non-aged phenotype in a cell.

In another embodiment, the loss-of-function, inhibitory, knock-out, gain-of-function or combinatorial screen comprises a whole-genome screen. Such whole-genome screens, also known as genome-wide screens, aim to elucidate the relationship between genotype and phenotype by altering the expression or activity of a gene or gene product on a genome-wide scale and studying the resulting phenotypic alterations, such as in step iii) of the method described herein. As their name suggests, loss-of-function, inhibitory or knock-out screens perturb the expression of genes, preventing them from functioning as normal. Thus, when loss-of-function, inhibitory or knock-out screens are performed a gene or combination of genes will be identified as being involved in the reversal of an ageing-phenotype when said ageing-phenotype persists for longer or is not reversed in an artificially aged cell following induction of ageing compared to an artificially aged cell in which no loss-of-function, inhibition or knock-out of the gene or combination of genes has occurred. Alternatively, when loss-of-function, inhibitory or knock-out screens are performed a gene or combination of genes will be identified as being involved in the maintenance of a non-aged phenotype when said phenotype is lost in an artificially aged cell, i.e. the cell displays an aged-phenotype, following induction of ageing compared to an artificially aged cell in which no loss-of-function, inhibition or knock-out of the gene or combination of genes has occurred. Gain-of-function screens enhance the activity or expression of a gene or gene product. When gain-of-function screens are used a gene or combination of genes will be identified as being involved in the reversal of an ageing-phenotype when said ageing-phenotype is reversed more quickly or persists for a shorter period of time in an artificially aged cell following induction of ageing compared to an artificially aged cell in which no gain-of-function of the gene or combination of genes has occurred. Alternatively, a gene or combination of genes will be identified as being involved in the maintenance of a non-aged phenotype when said phenotype persists in an artificially aged cell, i.e. the cell does not display an aged phenotype, following induction of ageing compared to an artificially aged cell in which no gain-of-function of the gene or combination of genes has occurred. A combinatorial screen comprises a combination of one or more of loss-of-function, inhibitory, knock-out and gain-of-function as described herein. Such combinatorial screens allow the accurate identification of combinations of genes involved in the reversal of an ageing phenotype or maintenance of a non-aged phenotype.

Methods of performing whole-genome screens are known in the art and any such method will be appreciated to find utility in the methods described herein. However, of particular utility are screens performed using CRISPR as described herein. Thus, in one embodiment, the loss-of-function, inhibitory, knock-out, gain-of-function or combinatorial screen is performed using CRISPR. In a further embodiment, wherein the screen is a loss-of-function or inhibitory screen the CRISPR is CRISPRi. CRISPRi can sterically repress transcription by blocking either transcriptional initiation or elongation. This is accomplished by designing single guide RNA (sgRNA) complementary to the promoter or exonic sequences of the gene to be transcriptionally inhibited or repressed. Depending on the nature of the CRISPR effector, targeting either the template or non-template strand leads to stronger repression. For example, with dCas9, repression is stronger when the guide RNA (gRNA) is complementary to the non-template strand. Unlike transcription elongation block, inhibition/repression by CRISPRi is independent of the targeted DNA strand when targeting the transcriptional start site. In a yet further embodiment, wherein the screen is a knock-out screen the CRISPR is CRISPR-ko. CRISPR-ko involves the insertion or deletion of bases upon repair of double stand DNA breaks created by the Cas9 nuclease in genes to which it has been targeted by a gRNA. Following creation of the double strand break, the imprecise non-homologous end joining repair (NHEJ) pathway is used but results in inserted or deleted bases, thus creating indels or producing gene knockouts. In another embodiment, wherein the screen is a gain-of-function screen the CRISPR is CRISPRa. CRISPR activation (CRISPRa) uses modified versions of CRISPR effectors which do not have endonuclease activity but comprise added transcriptional activators on dCas9 and/or the gRNAs. The transcriptional activators fused to the CRISPRa components therefore increase expression of genes of interest following targeting to the gene by the gRNA. In a still further embodiment, wherein the screen is a combinatorial screen the CRISPR is a combination of two or more of: CRISPRi, CRISPR-ko and CRISPRa, i.e. a combination of two or more of any of the above mentioned CRISPR techniques.

### Methods of Rejuvenation

In another aspect of the invention, there is provided a method of rejuvenating a somatic cell or a tissue-specific stem cell comprising altering the expression and/or activity of one or more gene identified by the methods described herein. It will be appreciated that references herein to “a” or “the” cell, such as a or the somatic cell, include a single or small number of cells, as well as to a population of cells, which may be large in number. Thus, it will be appreciated that any references herein, including any aspect or embodiment, to singular include plural and vice versa, unless explicitly stated otherwise.

The term “rejuvenating” as used herein refers to the reversal or maintenance of an ageing phenotype, such as those described herein. Such rejuvenation will therefore be appreciated to maintain a cell in a younger or non-aged stated compared to a cell in a non-rejuvenated state, or will alter the age of a cell so that it is younger or in a less-aged state than prior to rejuvenation. Whether a cell is “younger”, “non-aged” or “less-aged” may be determined using any ageing phenotype described herein, in particular using an epigenetic and/or transcriptomic clock. For example, a rejuvenated somatic cell or tissue-specific stem cell may display fewer alterations in cellular and/or nuclear morphology compared to cell in a non-rejuvenated state. Therefore, in one embodiment the rejuvenated somatic cell or tissue-specific stem cell comprises a methylation age (e.g. as determined using an epigenetic clock) of younger or less than prior to rejuvenation or than a somatic cell/tissue-specific stem cell in a non-rejuvenated state. In a further embodiment, the rejuvenated somatic cell or tissue-specific stem cell comprises a transcriptomic age (e.g. as determined using a transcriptomic clock) of younger or less than prior to rejuvenation or than a somatic cell/tissue-specific stem cell in a non-rejuvenated state. In another embodiment, the younger or less-aged methylation age corresponds to that of a somatic cell/tissue-specific stem cell from an earlier point in the life cycle of the tissue or organism from which the somatic cell was obtained. In a further embodiment, the younger or less-aged transcriptomic age corresponds to that of a somatic cell/tissue-specific stem cell from an earlier point in the life cycle of the tissue or organism from which the somatic cell was obtained. In yet further embodiments, the rejuvenated somatic cell or tissue-specific stem cell comprises, with respect to an ageing phenotype as described herein, the phenotype of a reprogrammed stem cell, such as a pluripotent stem cell and/or an iPSC. In some embodiments, the rejuvenated somatic cell or tissue-specific stem cell comprises a methylation age or a transcriptomic age corresponding to that of a reprogrammed stem cell, such as a pluripotent stem cell and/or an iPSC. Thus, in one embodiment the rejuvenated somatic cell or tissue-specific stem cell comprises a methylation age or a transcriptomic age of a pluripotent stem cell. In a further embodiment, the rejuvenated somatic cell or tissue-specific stem cell comprises a methylation age or a transcriptomic age of an iPSC. In a yet further embodiment, the rejuvenated somatic cell or tissue-specific stem cell comprises a methylation age and a transcriptomic age of pluripotent stem cell. In a still further embodiment, the rejuvenated somatic cell or tissue-specific stem cell comprises a methylation age and a transcriptomic age of an iPSC.

Any method known in the art for modulating and/or altering the expression and/or activity of one or more gene or combination of genes identified by the methods described herein may be used. For example, wherein expression and/or activity is to be increased, induction of expression/activity using one or more expression cassettes or by introduction of nucleic acids, polypeptides or small molecules (e.g. activatory/agonist polypeptides or small molecules) as described herein may be utilised. Alternatively, wherein expression and/or activity is to be reduced and/or repressed, any inhibitory small molecule, antagonist polypeptide, interference nucleic acids or interference/knock-out gene editing methods may be used. Such methods and approaches are known in the art and described herein. In particular embodiments, the expression of one or more gene or combination of genes identified by the methods described herein is altered using CRISPR, such as by CRISPRi and/or CRISPRa. It will be appreciated that, in the context of a combination of genes, expression of one gene or set of genes may be increased while expression of another gene or set of genes is inhibited/repressed. In one embodiment, the expression of one or more gene or combination of genes found to be involved in the reversal of an ageing phenotype or in the maintenance of a non-aged phenotype (i.e. to promote/positively affect said reversal or maintenance and promote a young, non-aged or rejuvenated state) is increased, such as activated or enhanced. In a further embodiment, expression is increased using CRISPRa. In another embodiment, the expression of one or more gene or combination of genes found to promote an ageing phenotype or accelerate the loss of a non-aged phenotype (i.e. to promote ageing) is decreased and/or inhibited. In a still further embodiment, expression is decreased and/or inhibited using CRISPRi and/or CRISPR-ko. In some embodiments, expression of a combination of genes is increased (e.g. activated or enhanced) and decreased and/or inhibited.

In some embodiments, the rejuvenated somatic cell comprises a reduced and/or slowed progression of an ageing phenotype compared to a non-rejuvenated somatic cell, wherein the ageing phenotype is as described herein. In a further embodiment, the rejuvenated somatic cell does not comprise an ageing phenotype found in a somatic cell in a non-rejuvenated state. For example, the somatic cell may comprise an ageing phenotype as described herein prior to rejuvenation and said ageing phenotype is eliminated following rejuvenation.

In some embodiments, the somatic cell is a neuron, glia cell, blood cell, immune cell, liver cell, hepatocyte, muscle cell, myocyte, fibroblast, skin cell, bone cell, cartilage cell, epithelial cell, endothelial cell or adipocyte. In further embodiments, the somatic cell is derived from an iPSC by forward programming as described herein.

It will be readily understood that the methods and uses described herein, or any/all steps thereof, may be performed in vivo, ex vivo or in vitro. In a particular embodiment, the methods and uses described herein, or any/all steps thereof, are performed in vitro, such as ex vivo.

Unless defined otherwise, all technical and scientific terms used herein have the meaning commonly understood by a person skilled in the art to which this invention belongs. As used herein, the term “about” when used herein includes up to and including 10% greater and up to and including 10% lower than the value specified, suitably up to and including 5% greater and up to and including 5% lower than the value specified, especially the value specified. The term “between” as used herein includes the values of the specified boundaries.

It will be understood that all embodiments described herein may be applied to all aspects of the invention and vice versa.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art.

### CLAUSES

A set of clauses defining the invention, its aspects and embodiments is as follows:

1. A method of transiently inducing ageing in a stem cell, said method comprising:
(i) introducing one or more ageing-inducing factor into the cell or exposing the cell to one or more exogenous ageing-inducing factor for a period of time, and
(ii) removing or reducing the one or more ageing-inducing factor, thereby leading to a reversal of one or more ageing phenotype in the cell.

2. The method of clause 1, wherein the stem cell is a pluripotent stem cell, an induced pluripotent stem cell (iPSC), a germline stem cell, a multipotent stem cell, an oligopotent stem cell, a unipotent stem cell or a tissue-resident stem cell.

3. A method of transiently inducing ageing in a somatic cell derived from an induced pluripotent stem cell (iPSC) by forward programming, said method comprising:
(i) introducing one or more ageing-inducing factor into the cell or exposing the cell to one or more exogenous ageing-inducing factor for a period of time prior to or after forward programming of the iPSC, and
(ii) removing or reducing the one or more ageing-inducing factor, thereby leading to a reversal of one or more ageing phenotype in the cell.

4. The method of clause 3, wherein the somatic cell is a neuron, glia cell, blood cell, immune cell, liver cell, muscle cell, myocyte, fibroblast, skin cell, bone cell, cartilage cell, epithelial cell, endothelial cell or adipocyte.

5. The method of any one of clauses 1 to 4, wherein introducing one or more ageing-inducing factor in step (i) comprises one or more expression cassette comprising a sequence encoding the one or more ageing-inducing factor.

6 The method of clause 5, wherein the one or more expression cassette is transiently transfected into the cell.

7. The method of clause 5, wherein the expression cassette and a gene encoding a transcriptional regulator protein are inserted into a genetic safe harbour site,
wherein the expression cassette further comprises an inducible promoter which is regulated by the transcriptional regulator protein.

8. The method of clause 5, wherein a gene encoding a transcriptional regulator protein is inserted into a first genetic safe harbour site and the expression cassette is inserted into a second genetic safe harbour site,
wherein the expression cassette further comprises an inducible promoter which is regulated by the transcriptional regulator protein, and
wherein the first and second genetic safe harbour sites are different.

9. The method of any one of clauses 5 to 8, wherein expression of the sequence encoding the ageing-inducing factor or the activity of the transcriptional regulator protein is controlled by an exogenously supplied substance, such as tetracycline and derivatives thereof or cumate.

10. The method of any one of clauses 1 to 7, wherein the one or more ageing-inducing factor is an alternative splice form of Lamin A, such as progerin.

11. The method of any one of clauses 1 to 10, wherein the one or more ageing-inducing factor is a mitochondrial DNA mutation, such as deletion of DNA polymerase subunit gamma (POLG).

12. The method of any one of clauses 1 to 11, wherein the one or more ageing-inducing factor is an ageing-inducing compound, such as a telomerase inhibitor, reactive oxygen species (ROS) or a compound that disrupts or stresses mitochondria function, such as 6-hydroxydopamine (6-OHDA), valinomycin, CCCP, rotenone or hydrogen peroxide.

13. The method of any one of clauses 1 to 12, wherein the one or more ageing-inducing factor is an inducer of DNA damage, such as ionizing radiation, platinum-containing drugs, cyclophosphamide, chlorambucil or temozolomide.

14. An artificially aged stem cell or somatic cell derived from an iPSC by forward programming produced by the method of any one of clauses 1 to 13.

15. Use of the artificially aged stem cell and/or somatic cell derived from an iPSC by forward programming of clause 14 in a method of identifying a gene or a combination of genes involved in the reversal of an ageing phenotype or in the maintenance of a non-aged phenotype in the cell.

16. A method of identifying a gene or a combination of genes involved in the reversal of an ageing phenotype or in the maintenance of a non-aged phenotype in a cell, said method comprising the steps of:
i) introducing one or more ageing-inducing factor into the cell or exposing the cell to one or more exogenous ageing-inducing factor for a period of time;
ii) optionally performing a loss-of-function, inhibitory, knock-out, gain-of-function or combinatorial screen in the cell;
iii) measuring an ageing phenotype of the cell; and
iv) identifying a gene or a combination of genes as being involved in the reversal of an ageing phenotype or in the maintenance of a non-aged phenotype when the ageing phenotype is altered and/or its progression is altered in the cell when a loss-of-function, inhibitory, knock-out, gain-of-function or combinatorial screen is performed compared to when the cell is not subjected to a screen.

17. The method of clause 16, wherein the loss-of-function, inhibitory, knock-out, gain-of-function or combinatorial screen comprises a whole-genome screen.

18. The method of clause 16 or clause 17, wherein the loss-of-function, inhibitory, knock-out, gain-of-function or combinatorial screen is performed using CRISPR.

19. The method of clause 18, wherein the screen is a loss-of-function or inhibitory screen and the CRISPR is CRISPRi, a knock-out screen and the CRISPR is CRISPR-ko, a gain-of-function screen and the CRISPR is CRISPRa, or a combinatorial screen and the CRISPR is a combination of two or more of: CRISPRi, CRISPR-ko and CRISPRa.

20. The use of clause 15 or the method of any one of clauses 16 to 19, wherein the ageing phenotype of the cell is selected from one or more of: proliferation, senescence, changes in cell and/or nuclear morphology, mitochondrial function, changes in gene expression, upregulation of Lamin-A and/or Lamin-C nuclear protein, epigenetic marks associated with ageing, altered DNA or histone methylation, methylation entropy, DNA double strand breaks, telomere length, and a transcriptomic and/or epigenetic clock.

21. The use or method of clause 20, wherein the ageing phenotype is reduced proliferation and/or increased senescence,
wherein the changes in nuclear morphology are folding abnormalities, blebbing and/or loss of nuclear circularity,
wherein the mitochondrial function is reduced oxygen consumption and/or increased mitochondrial reactive oxygen species (ROS),
wherein the changes in gene expression are selected from one or more of: downregulation of somatic cell lineage identity genes, downregulation of mitochondrial genes, upregulation of apoptosis- and/or senescence-related genes, and upregulation of DNA damage-related genes,
wherein the epigenetic marks associated with ageing are selected from one or more of: reduced heterochromatin trimethylated H3K9 (H3K9me3), reduced HP1γ, and increased γH2AX,
wherein the telomere length is shortened, and/or
wherein the transcriptomic and/or epigenetic clock is a single cell transcriptomic and/or epigenetic clock.

22. The use or method of any one of clauses 15 to 21, wherein the cell is a neuron derived from an iPSC by forward programming, and optionally wherein the ageing phenotype is a phenotype of a neurological degenerative disease, such as reduced process density and/or connectivity, reduced average dendritic length, reduced neurite diameter, downregulation of neuronal marker genes or reduced electrophysiological activity.

23. A method of rejuvenating a somatic cell comprising altering the expression and/or activity of one or more gene identified by the use or method of any one of clauses 15 to 22.

24. The method of clause 23, wherein the rejuvenated somatic cell comprises a reduced and/or slowed progression of an ageing phenotype compared to a non-rejuvenated somatic cell, and wherein the ageing phenotype is as defined in clause 20 or clause 21.

25. The method or use of any one of clauses 1 to 13 or clauses 15 to 24, wherein the method or use is performed in vitro.

The invention will now be described using the following, non-limiting examples:

### EXAMPLES

### Example 1—Materials and Methods

hiPSC Culture

Human induced pluripotent stem cells Bob (A1ATD-iPSCs) (Yusa et al., 2011) and KOLF2 (HPSI0114i; Human Induced Pluripotent Stem Cell Initiative, www.hipsci.org) cultures were performed as previously described (Pawlowski et al. (2017); and Vallier 2011). Briefly, cells were plated on Geltrex coated culture dishes and cultured in essential E8 medium (Life Technologies). Cells were passaged in small clumps using ReLeSR (StemCell Technologies) every 6-7 days. In order to age NGN2-Progerin iPSCs, at a 60% confluency, cumtae (System Biosciences) was added to the medium for 5 days, after that cumate was withdraw and iPSCs were dissociated for induction.

### Gene Targeting Constructs and Molecular Cloning

For the initial construct based on the OPTi-OX system, the previously designed vector pR26_CAG-rtTA was used (Pawlowski et al. (2017) and WO 2018/096343, the sequences of which are hereby incorporated by reference). For the inducible AAVS1 targeting vector, GFP-Progerin (IDT) was cloned into the previously designed pAAV_TRE-NGN2 vector via a P2A site (WO 2018/096343, the sequence of which disclosed therein is hereby incorporated by reference), or to the pAAV_HS4 (courtesy of Prof. Cédric Ghevaert). To construct the dual inducible system, (OPTi-OX)², pR26_CAG-rtTA-CymR targeting vector was constructed by ligating the cumate repressor CymR (Addgene, plasmid 48099) via a P2A-site into our original pR26_CAGrtTA vector. For the inducible AAVS1 targeting vector, the original pAAV_TRE-NGN2 vector was used in order to construct the aging vector. GFP-Progerin and the cumate operator CuO (Addgene, plasmid 48099) were ligated by Gibson Assembly (New England Biolabs) after NGN2 fragment separated by a STOP codon and a polyA sequence.

### HEK 293T Culture and Transfection

Human embryonic kidney 293T (HEK 293T) cells were cultured in DMEM-high glucose supplemented with 5% fetal bovine serum (FBS). pR26_CAG-rtTA-CymR and pAAVS1_NGN2-Progerin targeting vectors were transiently co-transfected into the 293T cells in the presence of a lipofection agent (ThermoFisher Scientific). 2 million 293T cells were plated on each 6-well plate for the transfection. The following day, when cells had reached 50-70% confluency, a transfection mixture consisting of OPTI-MEM medium (Gibco), lipofection gent, the targeting vectors, the two gRNAs and the two ZFNs was prepared and added to the plated 293T cells. The following day, the cells were washed once carefully with PBS, then the medium was changed to DMEN-high glucose with 5% FBS in presence or absence of doxycycline and/or cumate. The following day, cells were visualized under EVOS FL microscope (Life Technologies).

### Gene Targeting

Targeting of the hROSA26 and AAVS1 loci was performed as described previously (Bertero et al. (2016), Pawlowski et al. (2017) and WO 2018/096343, the sequences and methods of which are hereby incorporated by reference). Targeting of the hROSA26 locus was by nucleofection. Neomycin-resistant colonies were picked and screened by genotyping. Correctly hROSA26-rtTA-CymR-targeted clones were subsequently targeted with the inducible transgene cassette in the AAVS1 locus by nucleofection. Resulting puromycin-resistant colonies were picked and re-analysed by genotyping.

Targeting of the hROSA26 locus (Irion et al. (2007)) was performed by nucleofection. Human induced PSCs were dissociated to single cells with Accutase (Gibco), and 2×10⁶ cells were nucleofected (100 μL reaction volume; total of 12 μg of DNA, which was equally divided between the two gRNA/Cas9n plasmids and the targeting vector) using the Lonza P3 Primary Cell 4D-Nucleofector X Kit and cycle CA-137 of the Lonza 4D-Nucleofector System. Nucleofected hiPSCs were plated onto Geltrex (Thermo Fisher Scientific) coated dish and cultured in essential E8 medium. Colne-R (StemCell Technologies) was added for 48h after nucleofection to promote cell survival. After 3-4 days, neomycin-resistant hiPSCs were selected by adding G418 (50 μg/mL, Sigma-Aldrich) for 7 days. Subsequently, individual clones were picked, expanded and finally analysed by genotyping.

Targeting of the AAVS1 locus (Hockemeyer et al. 2009) was performed by nucleofection similar as for the hROSA26 locus (100 μL reaction volume; total of 12 μg of DNA, which was equally divided between the two AAVS1 ZFN plasmids and the targeting vector). After 3-4 days, resistant hiPSCs were selected by adding puromycin (1 μg/mL, Sigma-Aldrich) for 4-5 days. Subsequently, individual clones were picked, expanded and analysed by genotyping.

Drug-resistant hiPSC clones from targeting experiments were screened by genomic PCR to verify site-specific transgene integration, to determine the number of targeted alleles, and to exclude off-target integrations. PCRs were performed with LongAmp Taq DNA Polymerase (New England Biolabs).

Generation of iNeurons and Aged iNeurons from hiPSCs

NGN2 and NGN2-Progerin iPSCs were dissociated into single cells with Accutase and plated onto PDL/Geltrex coated dishes at a density of 30K cells per cm². Forward programming was initiated 24 hours after the split. The induction was performed in DMEM/F-12 supplemented with Glutamax (100×), Non-Essential Amino Acids (100×), 50 μM 2-Mercaptoethanol (Gibco), 1% Penicillin/Streptomycin, 1 μg/mL Dox. After 2 days of induction, the medium was switched to Neurobasal-medium supplemented with Glutamax (100×), B27 (50×), 10 ng/mL BDNF (Peprotech), 10 ng/mL NT3 (R&D Systems), 1% Penicillin/Streptomycin, and 1 μg/mL Dox. In order to age NGN2-Progerin iNeurons (iNs), at D7 post induction, cumate was added to the medium for 7 days. Pre-aged iNs were induced as described above from 5 days aged NGN-Progerin iPSCs, no additional cumate was added during the induction process.

### Primary Rodent Astrocytes Culture for Mito Stress Test Assay

All experiments on animals or involving the use of animal tissues were performed in accordance with a project license held under the Animals (Scientific Procedures) Act 1986, Amendment Regulations 2012, following ethical review by the University of Cambridge animal welfare and ethical review body.

Primary mixed glial cultures were derived from the cerebral cortices of P0-P2 neonatal Spraque Dawley rats and were generated along the previous guidelines (McCarthy and De Vellis (1980)), with minor modifications (Syed et al. (2008)). Mixed glia cells were maintained for 10 days in culture after which flasks were shaken for 1 hour at 260 rpm on an orbital shaker to remove the loosely attached microglia, and then overnight at 260 rpm to dislodge oligodendrocyte precursors. Astrocyte cultures were then maintained in glial culture medium (Dulbecco's modified eagle's medium (DMEM) supplemented with 10% FBS, glutamine and 1% penicillin/streptomycin) for at least two weeks before passaging and platting with iNs for multi-electrode array recording.

### Immunocytochemistry

Cells were fixed in 4% paraformaldehyde (Alfa Aesar) for 15 minutes at room temperature and subsequently washed three times with PBS. The cells were then permeabilised with 0.1% Triton-X-100 for 15 min at room temperature. Then, blocked with 10% goat or donkey serum (abcam) and 0.3% Triton X-100 (diluted in PBS) for 30-45 minutes at room temperature. Subsequently, cells were incubated with appropriately diluted primary antibodies in 2% goat or donkey serum and 0.1% Triton X-100 (diluted in PBS) at 4° C. overnight. After three washes with PBS, the cells were incubated for 1 hour at room temperature with corresponding secondary antibodies in PBS supplemented with 1% goat or donkey serum. Nuclei were visualised with 4′,6-diamidino-2-phenylindole (DAPI, Thermo Fisher Scientific).

Images were acquired using a Zeiss LSM 700 confocal microscope (Leica). To quantify cellular fluorescence intensity, images were processed using ImageJ software (version 2.00, NIH). Corrected total cell fluorescence (CTCF) was assessed from cells for each of the tested markers and presented in bar graphs. The number of γH2Ax foci was calculated manually and was plotted into a bar graph.

### Neurite Measurements

Dendrite lengths were measured using ImageJ software with NeuronJ plugin to trace MAP2 labelled neurites and are presented in bar graphs. Neurite diameters were measured using ImageJ software and are presented in a scatter plot and in a Gaussian distribution graph using non-linear regression.

### Cell Mito Stress Test Assay

iNs, post-aged iNs and pre-aged iNs were induced for 3 days in culture, then dissociated into single cells using accutase and replated on Seahorse XFp Cell Culture Miniplates (Agilent). Induction was followed as described above. At day 14 post induction, prior to the experiment, induction medium was replaced by XF DMEM medium containing 10 mM glucose, 1 mM pyruvate and 2 mM L-Glutamine. A Cell Mito Stress test kit (Agilent) was used to assess mitochondrial function. Cells were treated with 10 μM oligomycin, 10 μM Carbonyl cyanide-4 (trifluoromethoxy)phenylhydrazone (FCCP) and 5M rotenone/antimycin A. Oxygen consumption rates (OCRs) were measured with a Seahorse XFp Extracellular Flux analyser (Agilent) at 37° C. using the Mito Stress Test program.

### Multi Electrode Array (MEA) Recordings

Non-aged (NGN2) iNs, post-aged and pre-aged (N2P) iNs were dissociated into single cells at day 3 post induction and replated on PDL/Geltrex coated wells of CytoView MEA 48 (Axion Biosystems) with or without rat cortical astrocytes, at a ratio of 1:1. Co-cultures were maintained in Neurobasal medium for the duration of the recordings. Recordings were performed at D14 and D21 post induction of iNeurons, using Maestro Pro MEA system (Axion Biosystems).

### RNA Extraction

Total RNA was extracted from hiPSCs and iNeurons for each condition using GenElute mammalian total RNA miniprep kit (Sigma-Aldrich) with On-Column DNasel Digestion Set (Sigma-Aldrich).

### Quantitative Real-Time PCR (RT-PCR)

cDNA synthesis was performed with the Maxima First Strand cDNA Synthesis Kit (Thermo Fisher Scientific). PowerUp SYBR Green Master Mix (Thermo Fisher Scientific) was used for RT-PCR. Samples were run on the QuantStudio 6 Flex Real-Timer PCR System machine (Applied Biosystems). All samples were analysed in technical duplicates and normalised to the house-keeping gene Porphobilinogen Deaminase 1 (PBGD1). Results were analysed with the ΔΔCt method.

### RNA Sequencing

Read pre-processing, QC, alignment, and gene quantification. Prior to differential expression (DE) analysis, raw reads were evaluated for quality check in terms of sequencing quality and contamination. Trimming was performed using BBDuk [from BBMap v38.76] (Bushnell, 2017). Reads were then aligned to latest reference genome (hg38) with GTF from Gencode (v33) using STAR aligner v2.7.1a (Dobin et al., 2013). Aligned reads were transformed into read counts per gene using RSEM v1.3.3 tool (Li & Dewey, 2011).

Differential expression analysis. Pairwise expression analysis was performed using DESeq2 R package (Love et al., 2014) with multiple combinations of samples on the basis of control, aged and iNs/iPSCs. Briefly, expression counts were scaled and normalized to correct the sequencing depth and batch differences among samples for the pairwise group. These normalized counts were then used for differential expression analysis and to generate fold change values in log2 scale [log2(sample2/sample1)] for contrasts. Genes with lower read count can generate higher fold change values, which may lead to possible false positives. Hence, to adjust the fold changes that are risen due to ratio between lower read counts in samples, the fold change shrinkage estimator approach from DESeq2 was employed. Genes with adjusted p.value ≤0.05 and log2fold change of +1 or −1 were considered as significantly up and down-regulated genes. Further, using desiR v1.2.1 R package (Lazic, 2015), genes were scored based on P-value, average TPM and logFC to prioritize the highly significant genes.

Annotation and interpretation. Principal component analysis (PCA) and volcano plots were generated using DESeq2 and ggplot2 (Wickham, 2016) for different groups and conditions. Venn diagrams were generated using VennDiagram v1.6.20 R package (H. Chen & Boutros, 2011) by calculating intersection/overlap of significant DE genes across various contrasts. Heatmaps for marker genes and Gene-Ontology (GO) specific genes were plotted using ComplexHeatmap v2.2.0 R package (Gu et al., 2016). GO analysis was performed on up and down regulated genes from different analysis with the EnrichR v2.1 R package (E. Y. Chen et al., 2013). Statistically enriched biological processes (adjusted P value <=0.05) were considered.

### DNA Methylation

Genomic DNA was extracted from the different samples using Qiagen DNeasy Blood and tissue kit according to the manufacturer protocol. Samples were sent for DNA methylation services by Illumina Infinium MethylationEPIC BeadChip array via Erasmus MC (University Medical Center Rotterdam, NL).

### Epigenetic Clock Analysis

Epigenetic clock analysis of the DNA Methylation raw data was performed by Steve Horvath group according to their previous published work (Horvath 2013).

### Transcriptomic Clock Analysis

Transcriptomic clock analysis of the RNA raw data was performed using a proprietary algorithm from ShiftBioscience Ltd.

### Entropy Analysis

Shannon entropy analysis was performed as previously described (Hannum et al. (2013), Molecular Cell). The following formula was used:

$$Entropy = \frac{1}{N * log ( \frac{1}{2} )} {\sum }_{i} [ MFi * log ( MFi ) + ( 1 - MFi ) * log ( 1 - MFi ) ] ,$$

where MFi is the methylation fraction of the i^{th} methylation marker and N is the number of markers.

### Statistical Analysis

Statistical analysis was performed with GraphPad Prism (v8). The number of replicates, the statistical test used, and the test results are described in the figure legends. Unless stated otherwise data is presented as mean±SEM. Two-group comparisons were analysed using Student's t tests. For comparisons between two groups, the unpaired student t-test was used. For multiple comparisons, the one-way analysis of variance (ANOVA) was used with Dunnett's or Tukey's multiple comparisons test.

### Engineering of a Cas9 Line for CRISPR Screens

Three transgenes were integrated into GSH by genome engineering. Firstly, a CAG promoter-driven rtTA cassette for doxycycline-controlled transactivation of a second recombinant transgene was integrated into the ROSA26 locus. Secondly, a fusion protein of GFP and Progerin under the control of doxycycline-responsive promoter was integrated into the AAVS1 locus. Lastly, the CAG promoter-driven full-length Cas9 protein was integrated into the CLYBL locus, upstream of a P2A ribosomal-skipping sequence and the fluorescent protein mCherry for verification of integration.

### CRISPR Screens

A CROP-seq screen (Datlinger et al. (2017) Nature Methods) was performed to ensure the genotype-phenotype linkage in the downstream analysis, i.e. changes of the transcriptome of single cells can be traced back to the presence of certain sgRNAs and thus, to the KO of genes. The engineered hiPSC line (KOLF2-GFP-Progerin-Cas9) was transduced with a lentivirus encoding an sgRNA library targeting 1,000 genes at a low MOI (MOI<0.1) to ensure single integration of individual sgRNAs. Subsequently, transduced cells were selected by puromycin treatment, which enabled the puromycin resistance cassette which is present in the CROP-seq vector. The engineered hiPSC line harbouring single gene knockouts was treated for 5 days with doxycycline to induce a progerin-mediated ageing phenotype, followed by 14 days of rejuvenation (i.e. removal of doxycycline). At the end of the time course, rejuvenated iPSCs were processed as single cells for subsequent scRNA-seq analysis using the 10× Genomics Chromium 5′ Chemistry.

### Identification of Gene Knockout Influencing Aging/Rejuvenation

To identify the influence of single gene knockouts on the ageing/rejuvenation of hiPSCs on a single-cell level, a linear discriminant analysis (LDA) using the ageing signature was performed to create an ageing scale. That is, the aging signature was used to create a continuous variable (i.e. the ageing score) describing how “aged” a single cell is on a transcriptional level. This ageing scale was integrated into a Uniform Manifold Approximation and Projection (UMAP) plot of all single cells processed within the CRISPR knockout screen. This scale was used to evaluate the effect of knockouts on the rejuvenation process compared to cells transduced with a non-targeting sgRNA (“non-targeting”) that served as a control.

### Example 2—Ageing and Rejuvenation of Aged hiPSCs

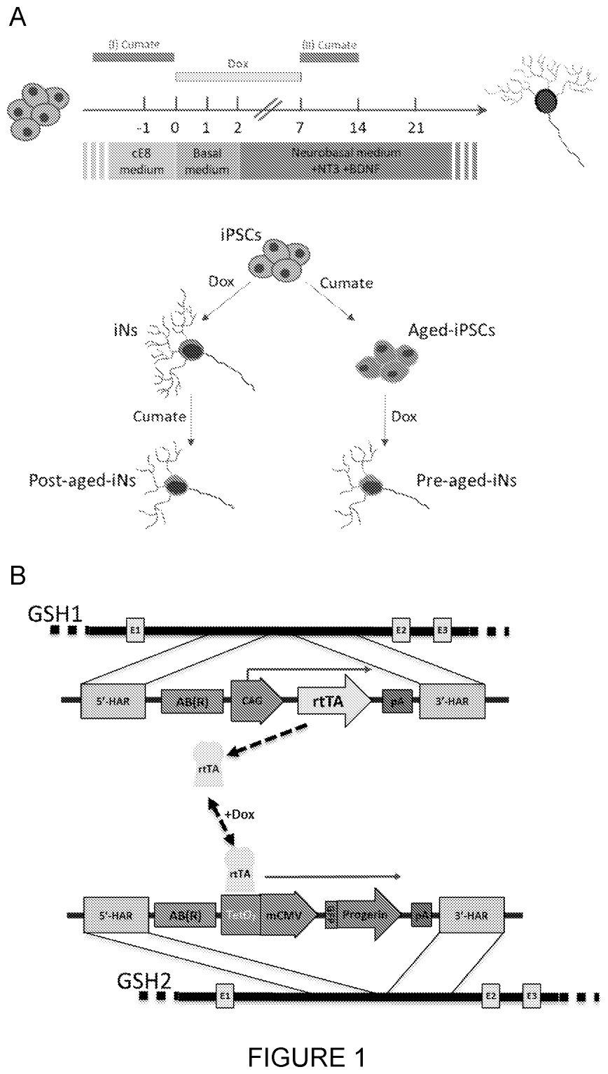

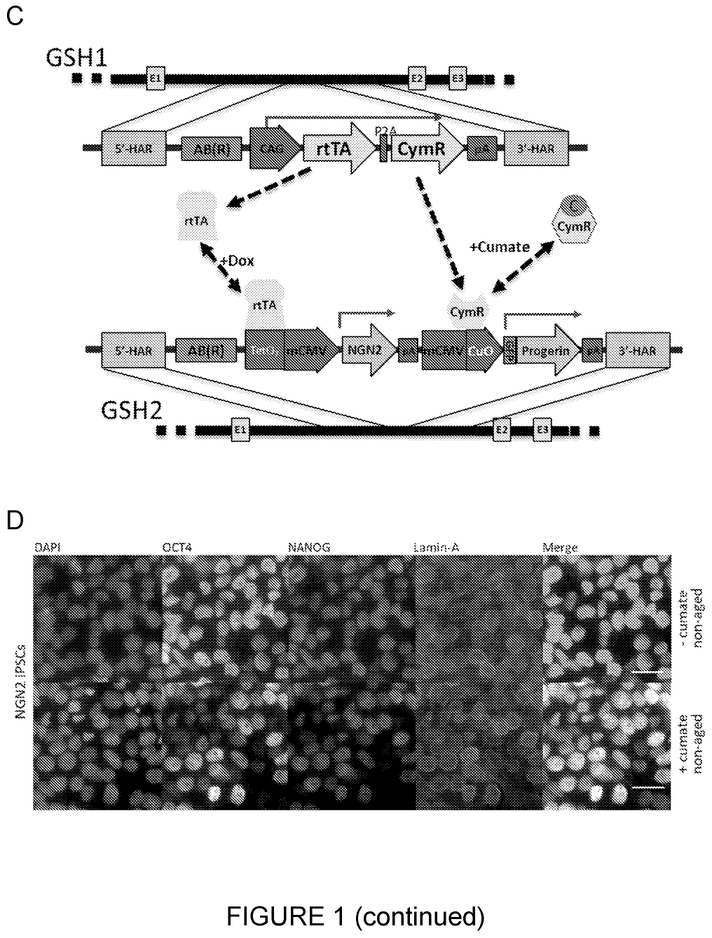

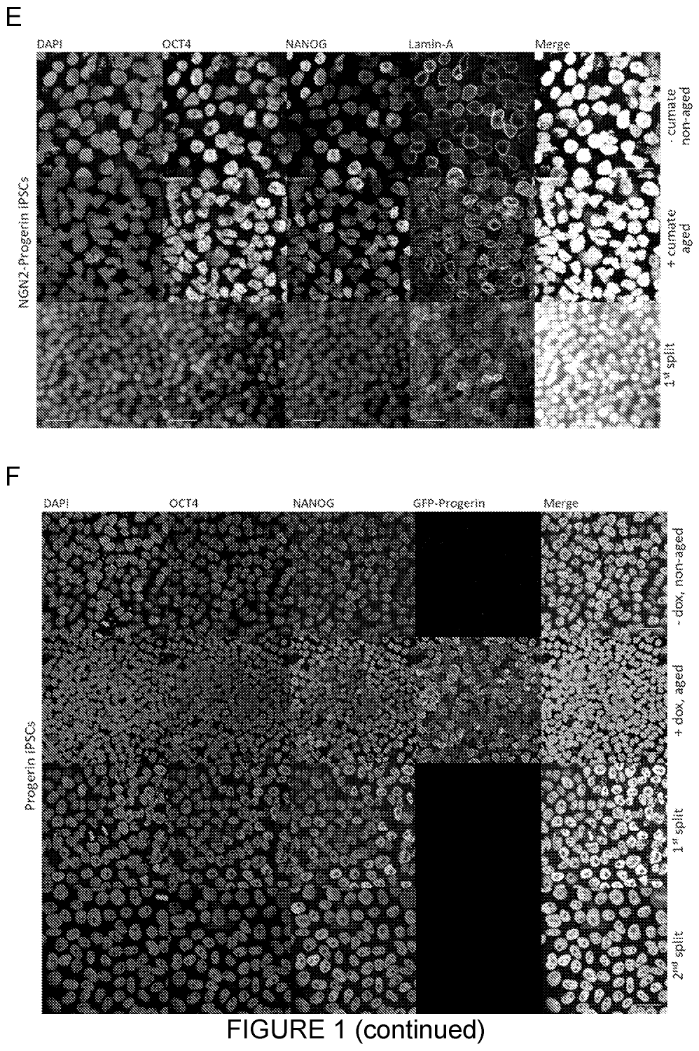

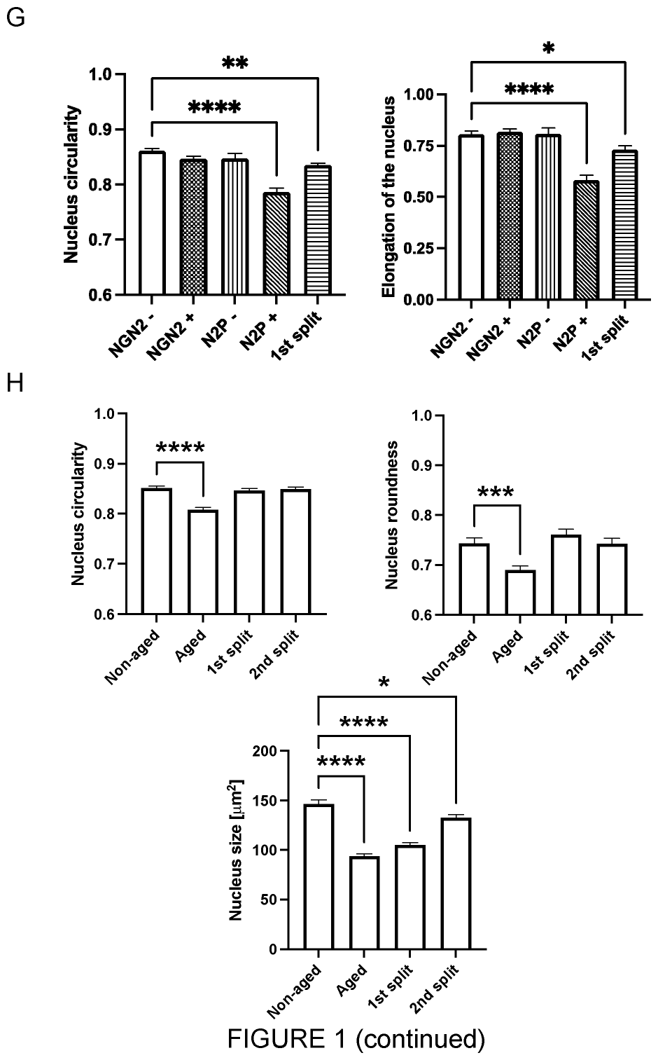

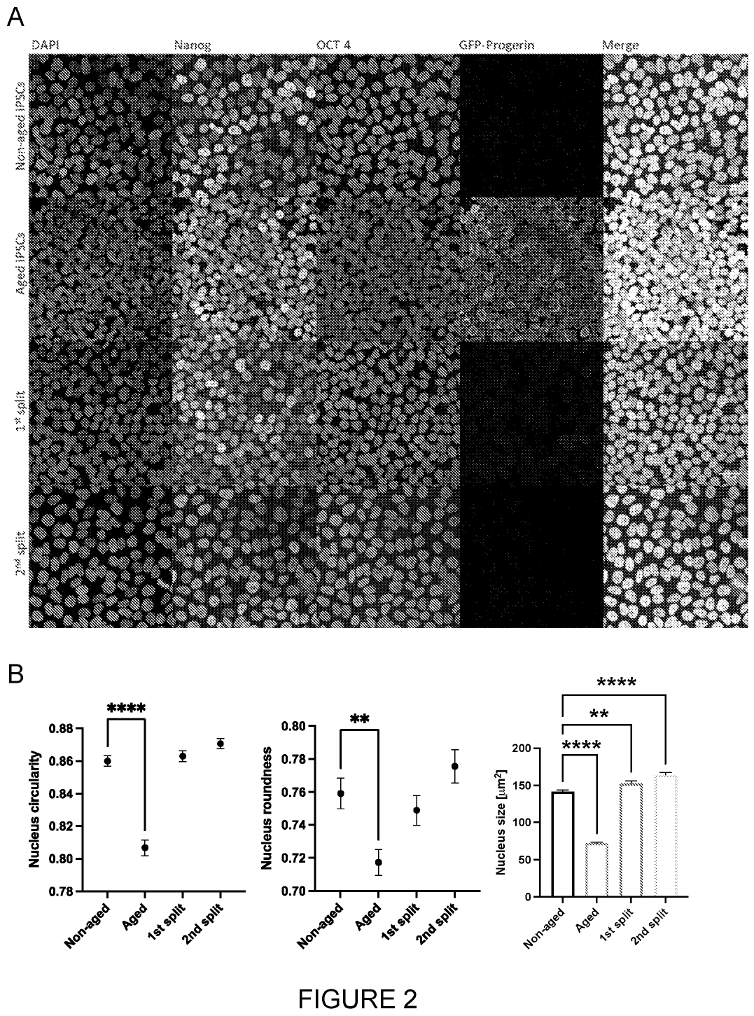

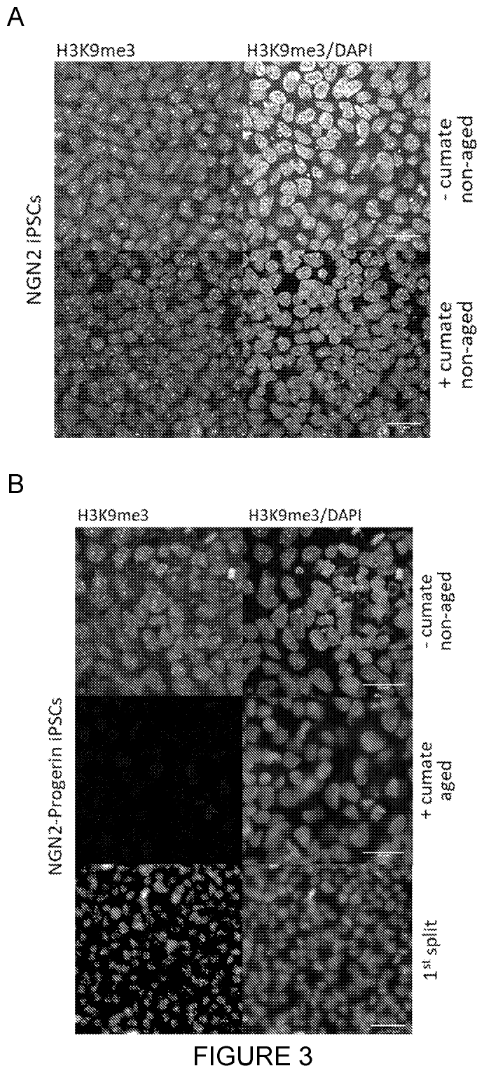

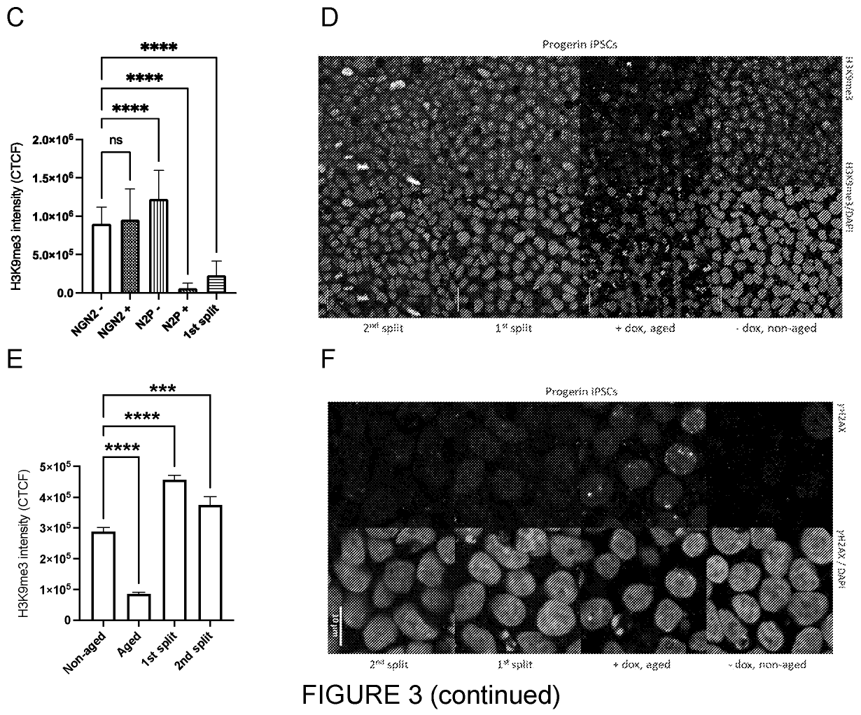

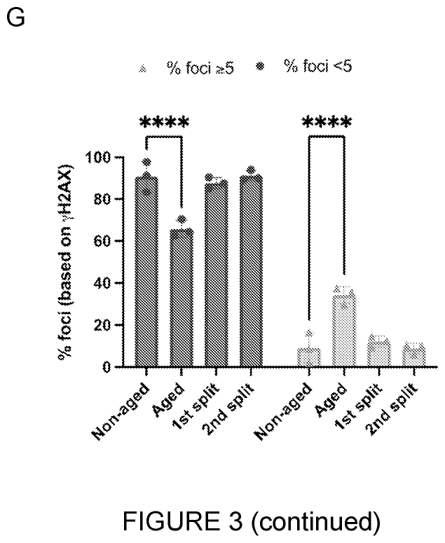

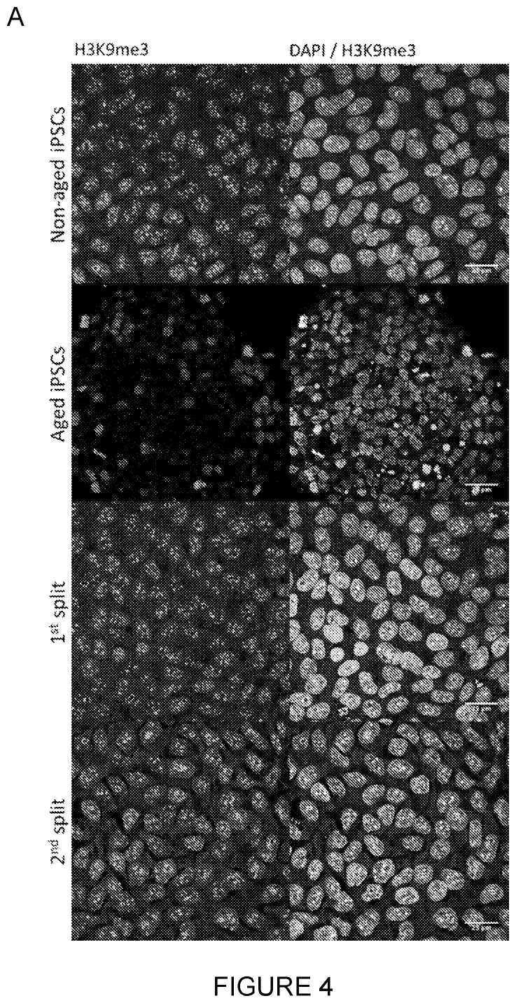

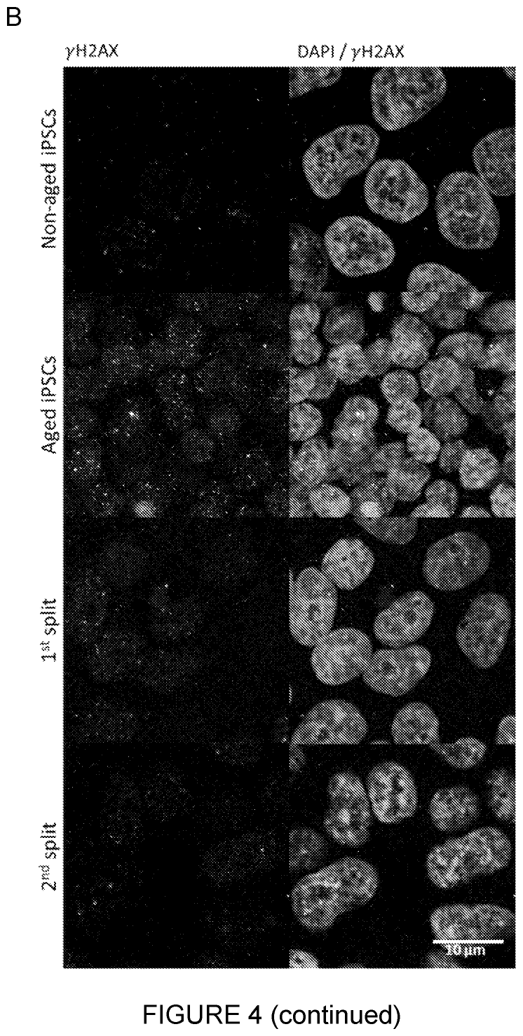

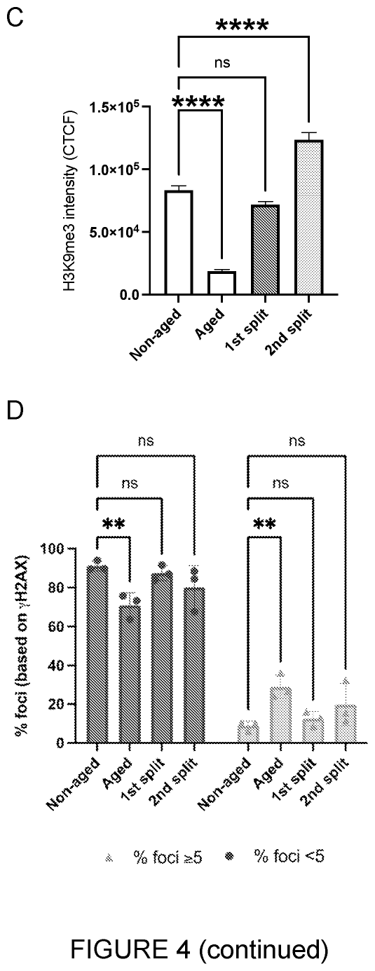

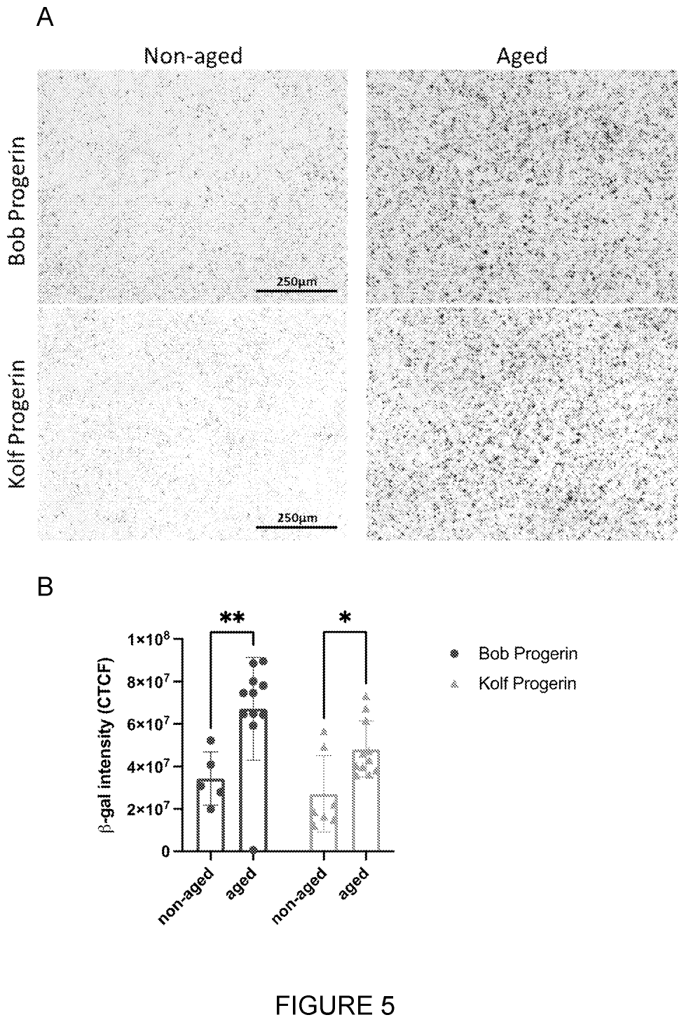

The iPSC^{N2P} line enabled the study of three ageing paradigms (FIG. 1A): 1) ‘pre-ageing’ of cells at the iPSC stage, that can be subsequently converted to neurons; 2) ‘post-ageing’ of cells at the iNeuron stage, and finally; 3) ageing of iPSCs without conversion into human iNeurons. One of the fundamental differences in this paradigm is that the cells are post-mitotic at the post-aged stage. It was therefore hypothesised that iPSCs and iNs may react differently to progerin expression and that this may lead to differences between the ‘pre-’ and ‘post-’ aged iNs. As iPSCs reset their ageing identity during reprogramming, it was tested whether the system could induce aging-related features in these cells, as a control iPSC^{NGN2} cells were used, which do not overexpress progerin. In addition, iPSCs expressing Progerin only under the TetOn system were also generated, referred to as Bob^{Progerin} and Kolf^{Progerin} (FIG. 1B). Induction of progerin over 5 days induced the typical morphological changes that have been observed in nuclei of HGPS patients. The aged iPSC^{N2P} and iPSC^{progerin} cells showed nuclear morphology abnormalities, such as folding, blebbing and loss of circularity compared to non-treated iPSC^{N2P/Progerin} cells or control iPSC^{NGN2} cells (FIGS. 1C-I and FIGS. 2A-B). In addition, the epigenetic marks associated with physiological ageing were studied. It was found that the aged iPSCs showed a significant loss of the heterochromatin trimethylated H3K9 (H3K9me3), which is commonly reduced with physiological ageing, and increase in DNA double-strand breaks (FIGS. 3 and 4). Another phenotype associated with ageing is senescence. Whether the aged iPSCs undergo senescence was tested by staining for the senescence-activated β-galactosidase (SA-β-gal) marker, which confirmed a significant increase in SA-β-gal for the aged iPSCs (FIG. 5). To test whether the cells maintained their ageing phenotype, the aged iPSCs were subjected to passaging. Following the first passage post ageing, the iPSCs seemed to rejuvenate and the ageing phenotypes were reversed, as can be seen from their normal nucleus morphology, levels of H3K9me3 expression and number of DNA double-strand breaks (FIGS. 1C-G, 2, 3 and 4).

### Example 3—Development of a Dual Inducible System (OPTI-Ox) 2, to Artificially Age iPSCs and iNeurons

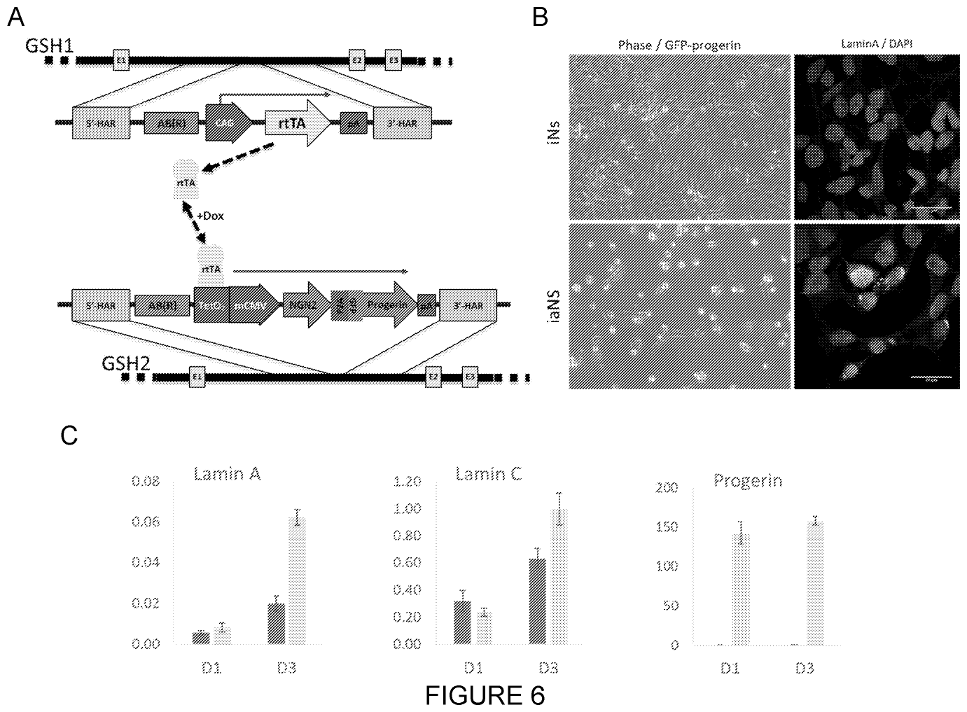

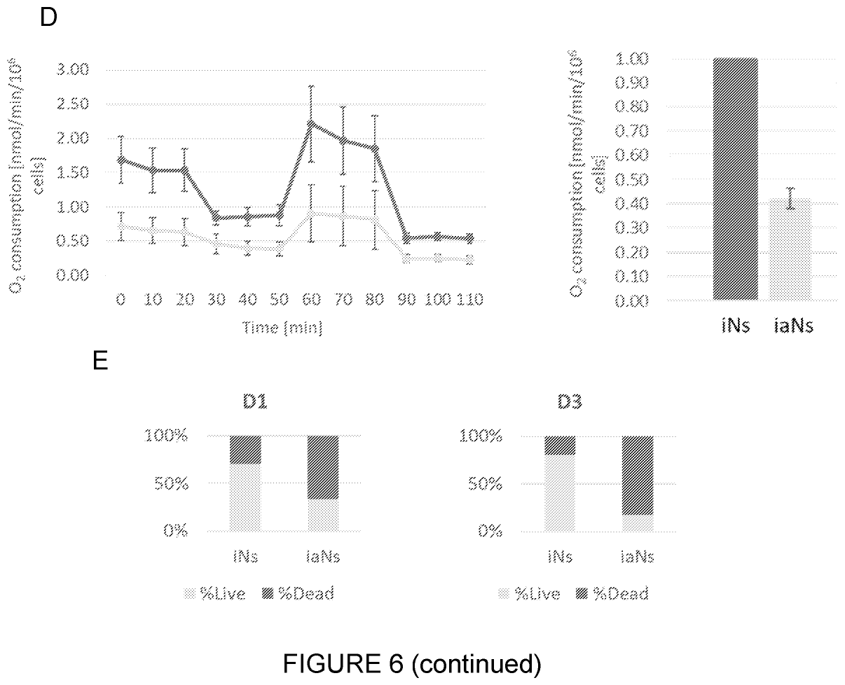

As a first proof of principle, a test paradigm using a single inducible gene switch based on a single cassette combining progerin and NGN2 expression was investigated. To that end, the previously published protocol for inducing neurons based on the OPTi-OX system was used. This system has proven to forward program hiPSCs into functional cortical neurons by overexpression of NGN2 from a genetically safe harbour (Pawlowski et al. (2017)). These induced neurons (iNeurons) represent a homogeneous population of glutamatergic neurons and were extensively characterised, showing to express relevant neuronal markers and to possess electrophysiology properties (Lam et al. (2017); Pawlowski et al. (2017); Tourigny et al. (2018); and Zhang et al. (2013)). A line of iNeurons (iNs) expressing GFP fused to progerin was generated to enable following progerin expression in the cells. In this cassette the reprogramming factor and progerin are linked by a 2A sequence (FIG. 6A). This cassette ensured efficient transgene expression of progerin as soon as the reprogramming factor NGN2 was expressed. Progerin expression was found to be highly efficient in the resulting neurons. These iNs were shown to have artificial ageing phenotypes, such as changes in nuclear morphology and upregulation of Lamin-A and Lamin-C, compared to control NGN2-iNs (FIGS. 6B-C). In addition, mitochondrial dysfunction was observed as reflected by the reduction in oxygen consumption levels (FIG. 6D). However, cell viability was compromised at an early stage (FIG. 6E). Titration of the doxycycline concentration used to induce NGN2-progerin expression demonstrated that this is likely a result of the high levels of progerin that are generated via this approach (data not shown). Thus, it was hypothesised that a new system is required to express NGN2 and progerin at different time points which would be more beneficial.

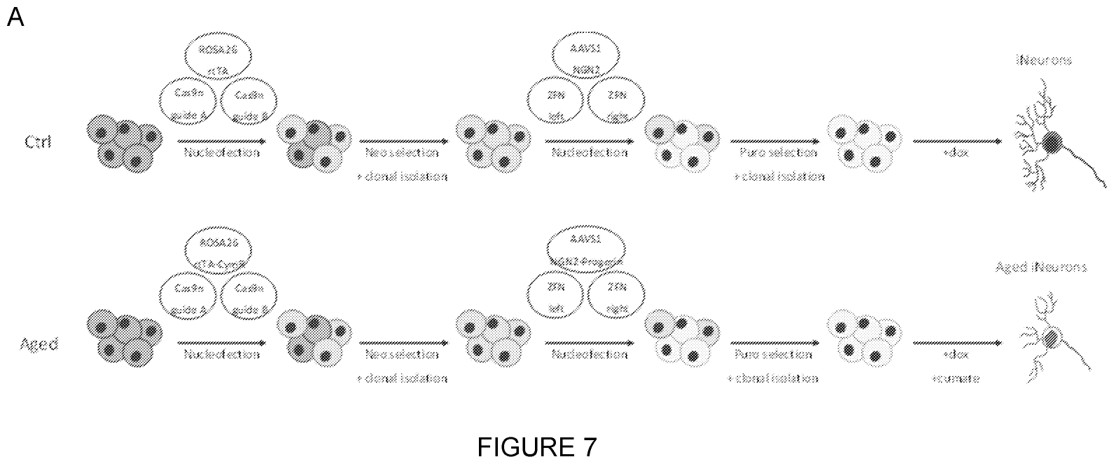

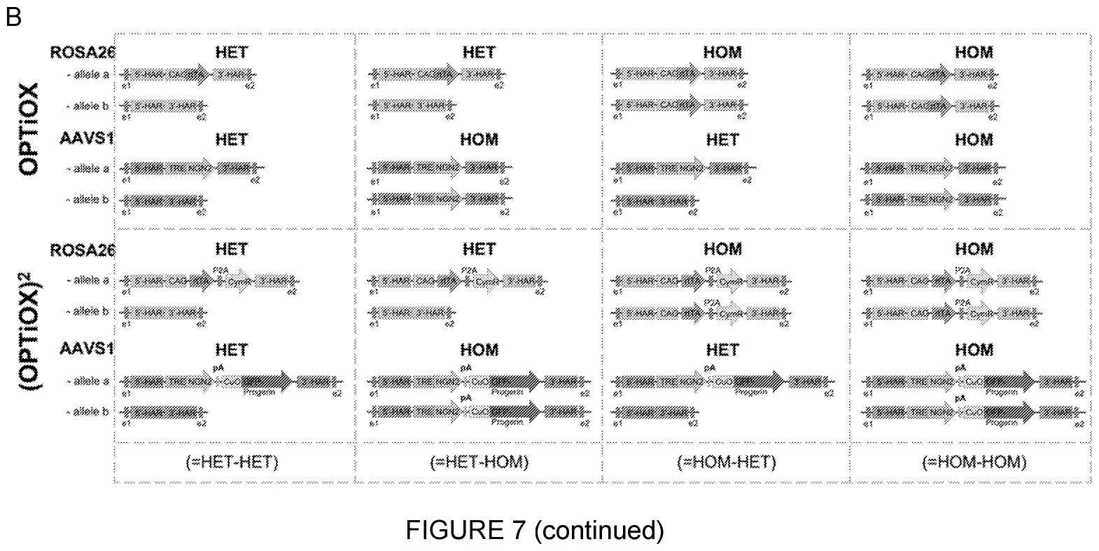

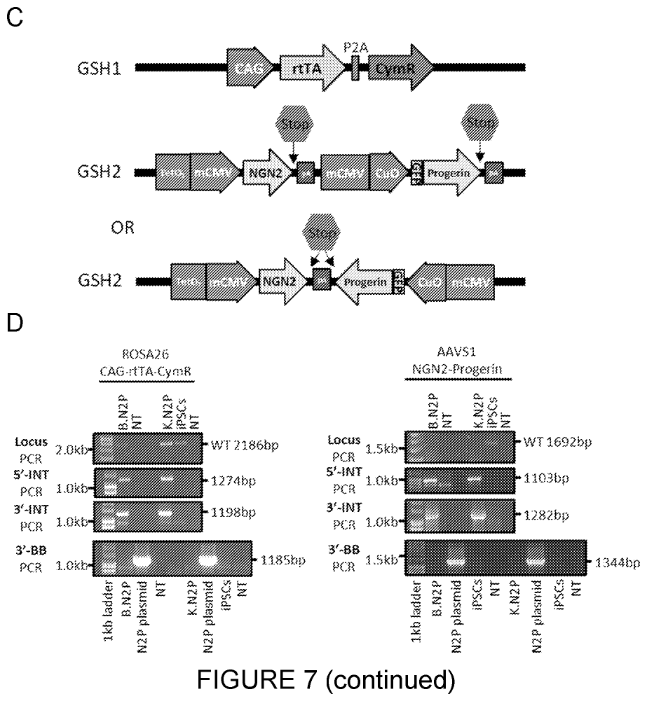

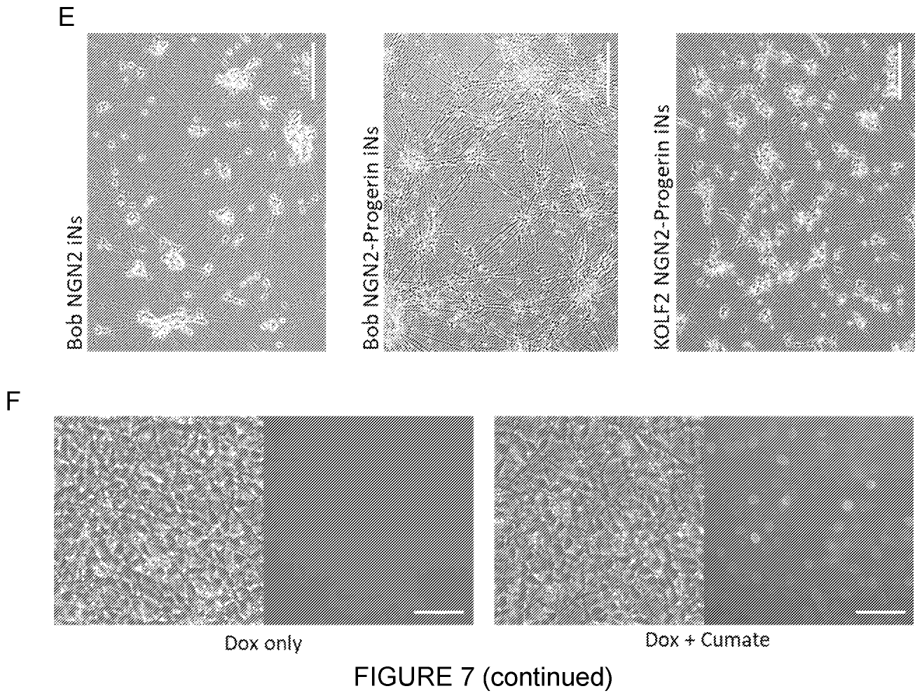

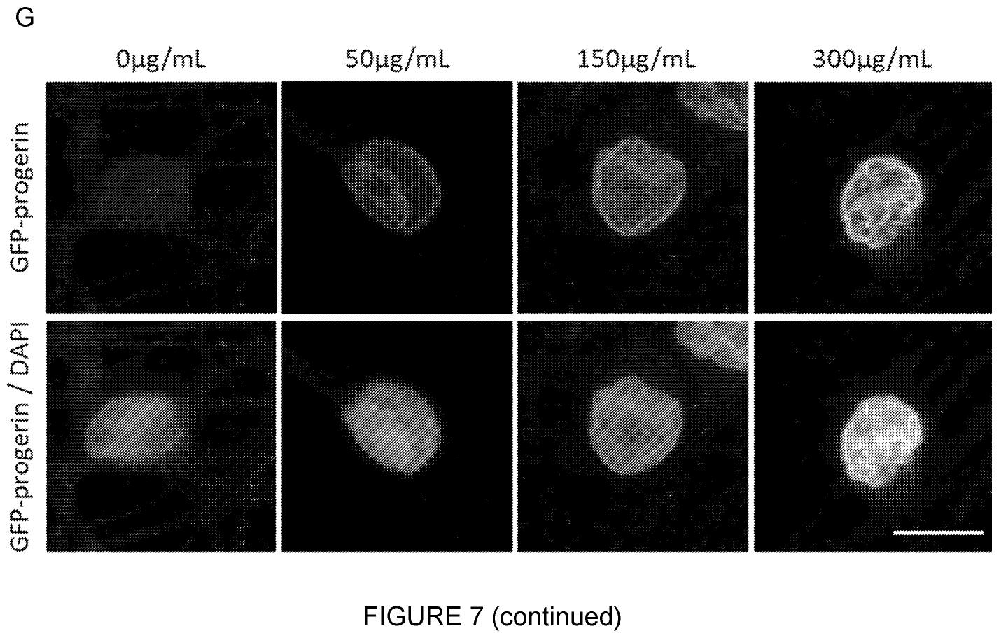

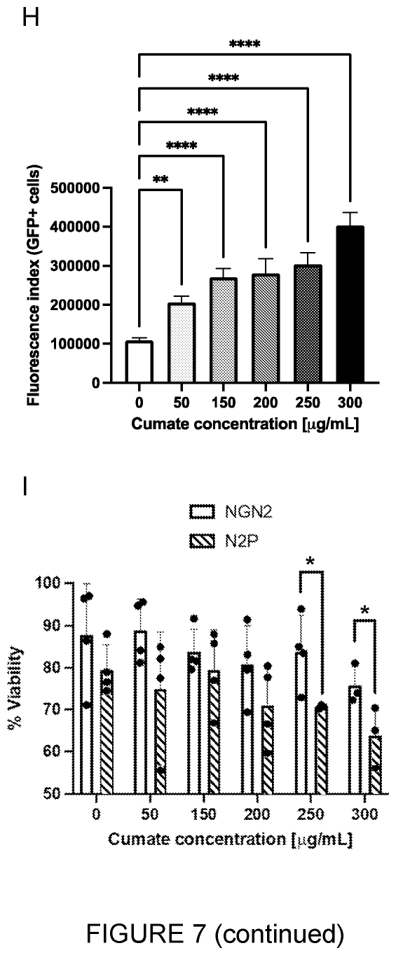

In order to allow for a separate control of NGN2 and progerin expression, the OPTi-OX technology was used and a dual system designed which is based on both the TetOn (Baron and Bujard (2000)) and the cumate switch (Mullick et al. (2006); Sato et al. (2020); and Seo and Schmidt-Dannert (2019)) inducible systems. This new system was referred to as (Opti-OX)². The cumate repressor (CymR) was first cloned into the original ROSA26 targeting vector, linked by a 2A sequence to rtTA (FIGS. 7A-F). Next, the cumate operator (CuO) followed by GFP-Progerin was cloned into the original targeting cassette of the cortical neurons (AAVS1-NGN2; FIGS. 7A-F). Initially, two constructs were tested for the AAVS1 locus. The first construct orients the cassettes in opposing directions to mitigate potential leakage from the NGN2 promoter (referred as ‘reverse’), whilst the second construct orients the progerin and NGN2 cassettes in the same direction (referred as ‘forward’; FIG. 7D). Both AAVS1 constructs, together with the rtTA-CymR construct, were transfected into HEK293 cells. The cells were then treated with cumate which demonstrated successful induction of progerin in AAVS1-NGN2-Progerin ‘forward’ but not ‘reverse’ transfected cells (data not shown). Following this, rtTA-CymR and AAVS1-NGN2-Progerin ‘forward’ constructs were used to generate NGN-Progerin (N2P) line in both Bob and KOLF2 iPSCs backgrounds. Drug-resistant hiPSC clones from the two lines were screened by genomic PCR to verify site-specific transgene integration, to determine the number of targeted alleles, and to exclude off-target integrations (FIG. 7E). To assess whether the resulting iPSCN2P cells are able to generate neurons, doxycycline was added to the medium without activating the cumate system. Both lines showed similar development of neurons and formation of neuronal networks as for the original NGN2-iNs system (FIG. 7F). In addition, no differences were observed in NGN2 overexpression based on RT-PCR analysis (data not shown). In a substantial series of experiments, administration schedules and cumate concentration was varied in order to optimise progerin expression level (FIGS. 7G-J). GFP expression was detectable in neurons induced with both doxycycline and cumate but not in those induced with doxycycline only (FIG. 7H). Moreover, GFP expression could be titrated by adjusting the dose of cumate (FIG. 7I). A slight background expression was detectable from the cells with no cumate due to cells autofluorescence, as demonstrated in FIGS. 7H-I.

### Example 4—Pre- and Post-Aged iNeurons Exhibit Ageing Phenotypes

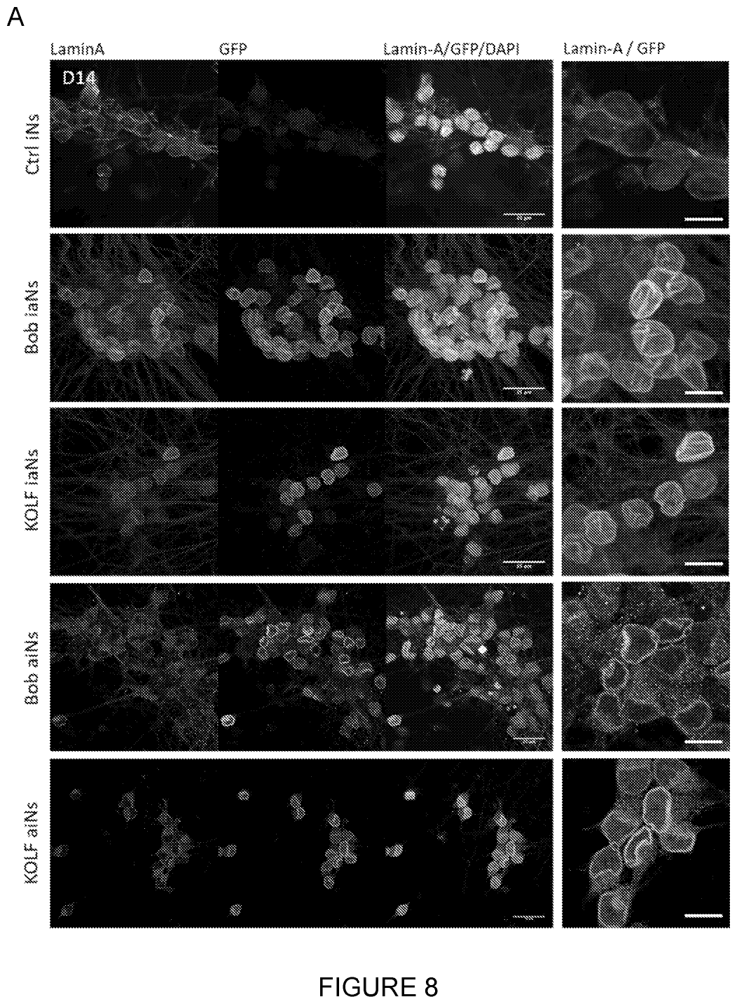

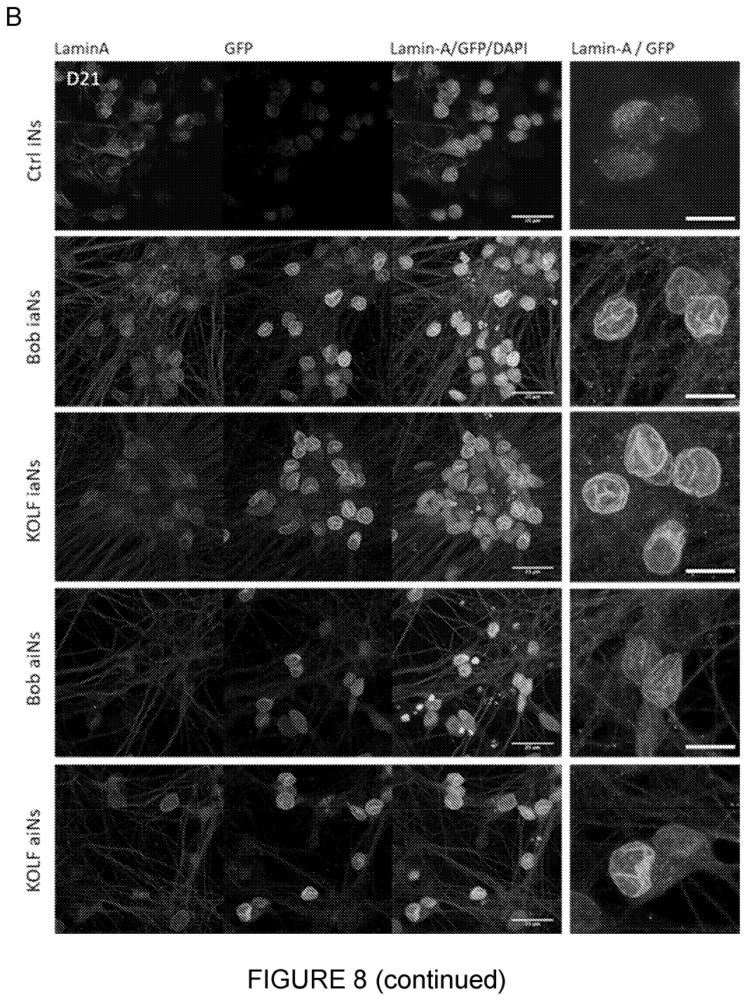

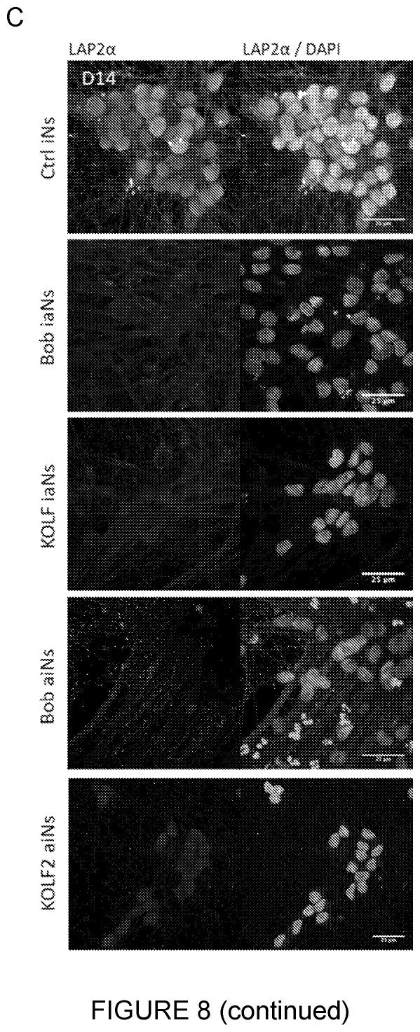

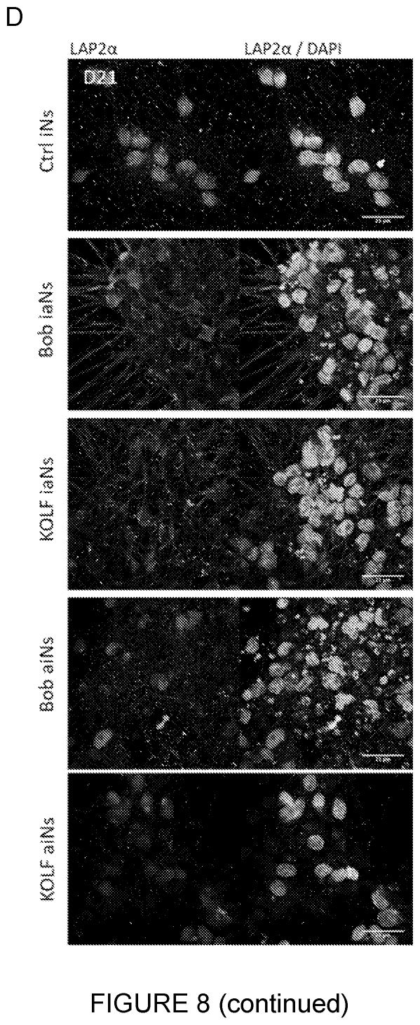

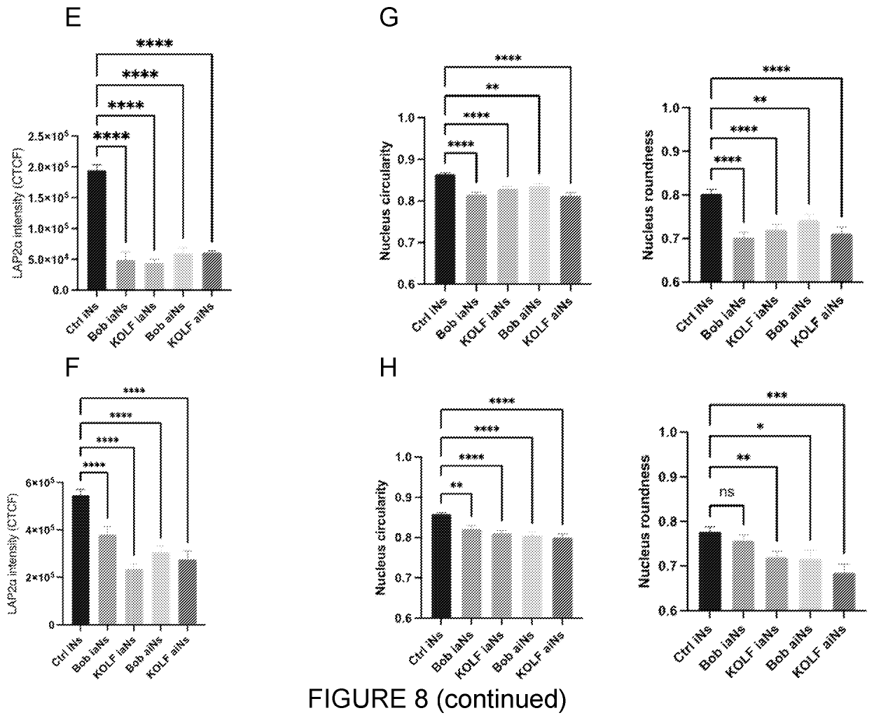

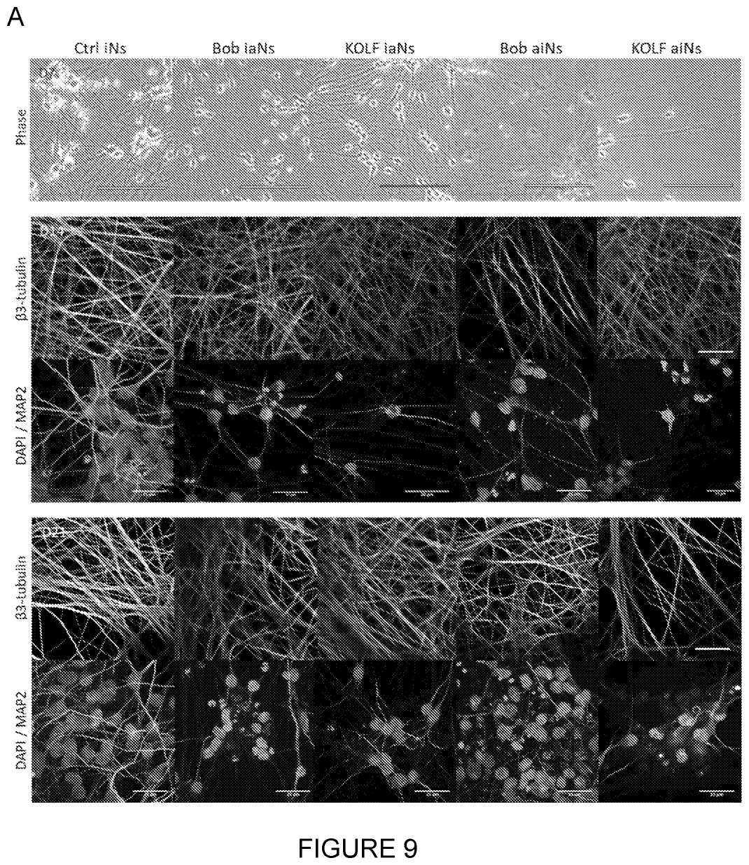

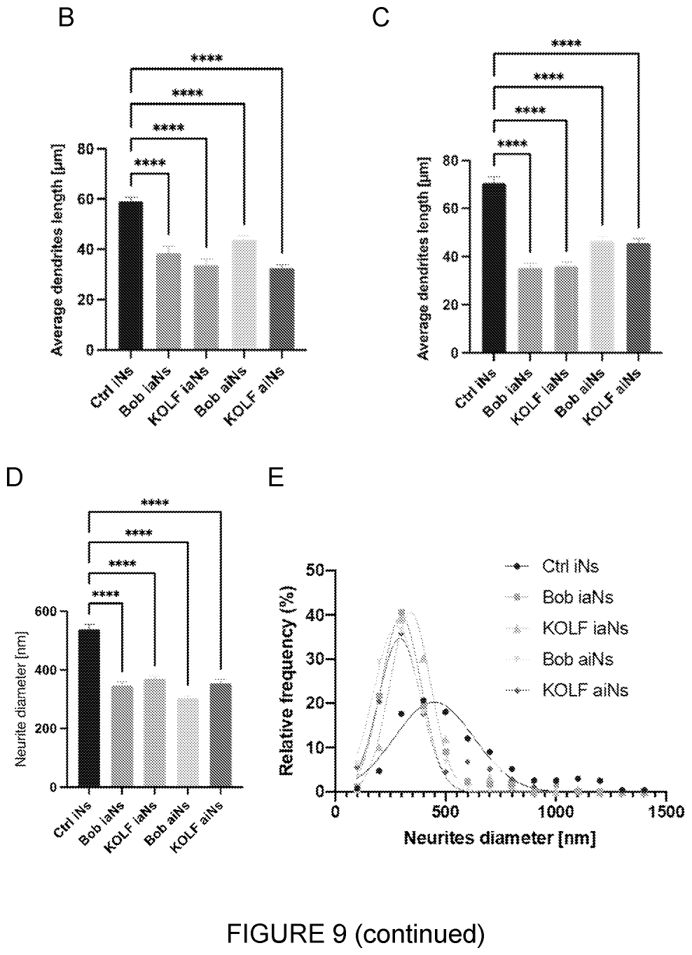

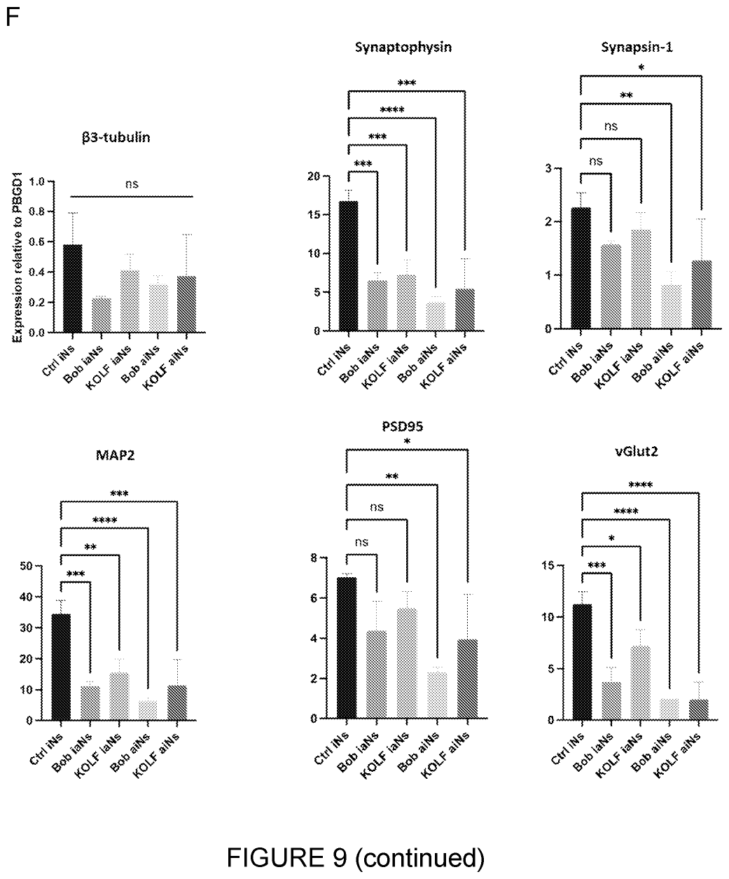

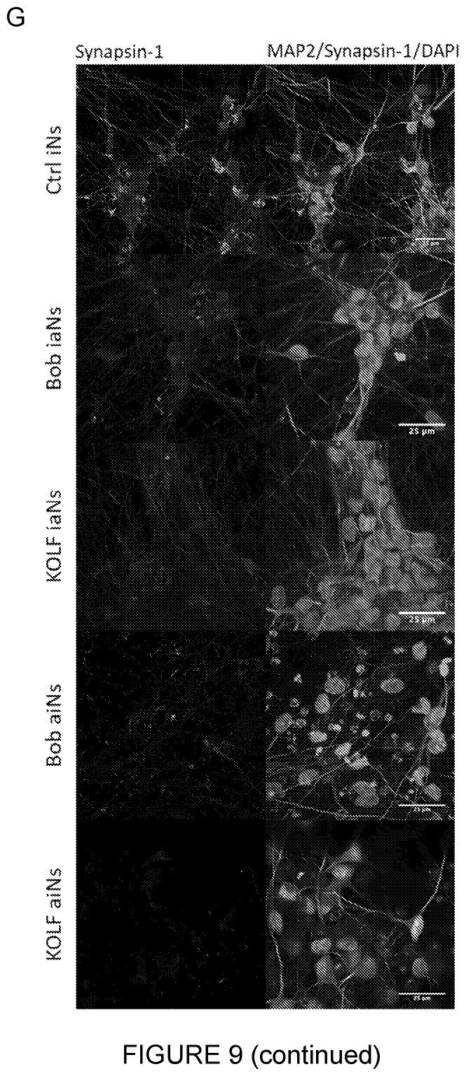

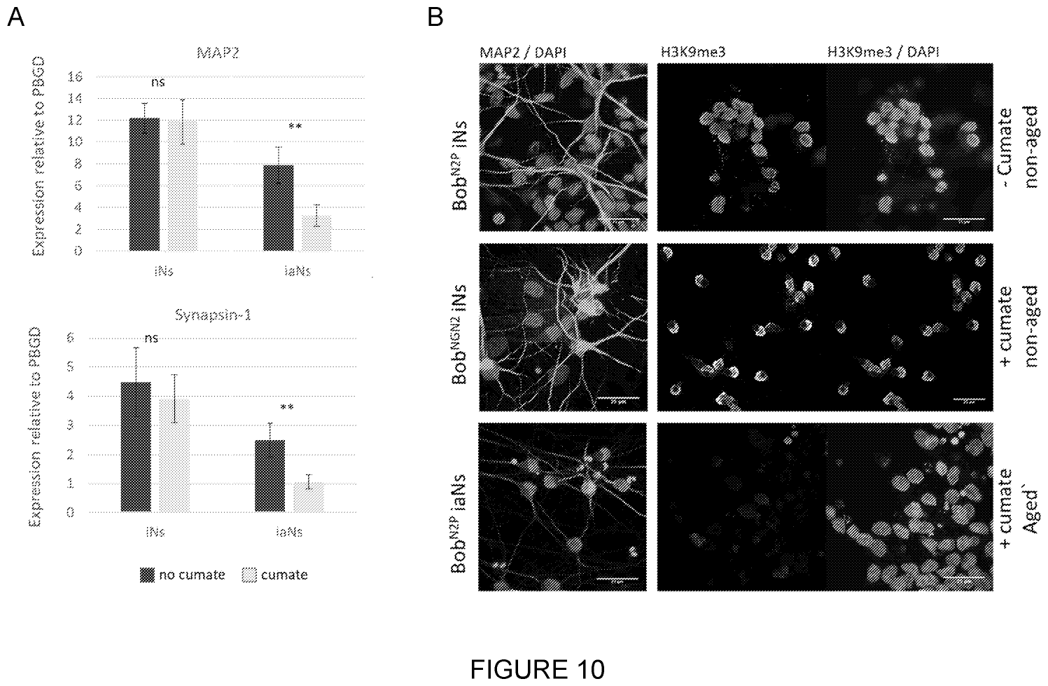

The other two paradigms were next tested, pre-aged iNs (aiNs) and post-aged iNs (iaNs) in parallel. To that end, Bob and KOLF IPSC^{N2P} targeted cells and iPSC^{NGN2} cells were treated with doxycycline for 7 days in chemically defined neuronal culture medium. For pre-aged iNs, iPSC^{N2P} cells were aged before inducing NGN2 expression as was described above. To initiate ageing in the post-aged group, cumate was added to the medium at day 7 post induction for a week. Following progerin overexpression, aged iNs (pre and post) showed evidence of enhanced nuclear folding and blebbing at both day 14 and day 21 post induction, based on GFP and Lamin-A expression while control iNs nuclei remained intact (FIGS. 8A-B). Interestingly, GFP expression was observed also for the pre-aged iNs at day 14 and day 21 post induction, even though they were treated with cumate during their iPSC stage only, suggesting on a slow turnover of progerin in the nuclear envelope. Immunostaining for the lamina associated protein LAP2a also showed a marked reduction in expression for both pre- and post-aged iNs compared to control iNs (FIGS. 8C-E and 8G). Similarly, as with the aged iPSCs, the nucleus morphology of the aged iNs was also found to be less circular and more elongated (FIGS. 8F and 8H). The parameters related to degenerative changes in neuronal network complexity were then examined. It was found that aged iNs displayed reduced process density and connectivity as compared to control non-aged iNs (FIG. 9A). The reduction in the complexity of processes was reflected by a marked reduction in average dendritic length (based on MAP2 expression) and by reduced neurite diameter for the aged iNs (FIGS. 9B-E). This was supported by RT-PCR quantitative analysis which showed downregulation in several neuronal markers (i.e. MAP2, vGLUT2, synapsin-1, synaptophysin and PSD95) for the aged iNs and by ICC for the synaptic marker synapsin-1 (FIGS. 9F-G). To eliminate the possibility of cumate by itself being responsible for these changes, RT-PCR analysis was performed for both iPSC^{NGN2}- and iPSC^{N2P}-derived iNs treated with and without cumate. These data confirmed that the reduction in neuronal markers was due to progerin overexpression and not due to the exposure to the cumate molecule (FIG. 10).

### Example 5—Peripheral Heterochromatic Marker Expression and DNA Damage in Aged iNeurons

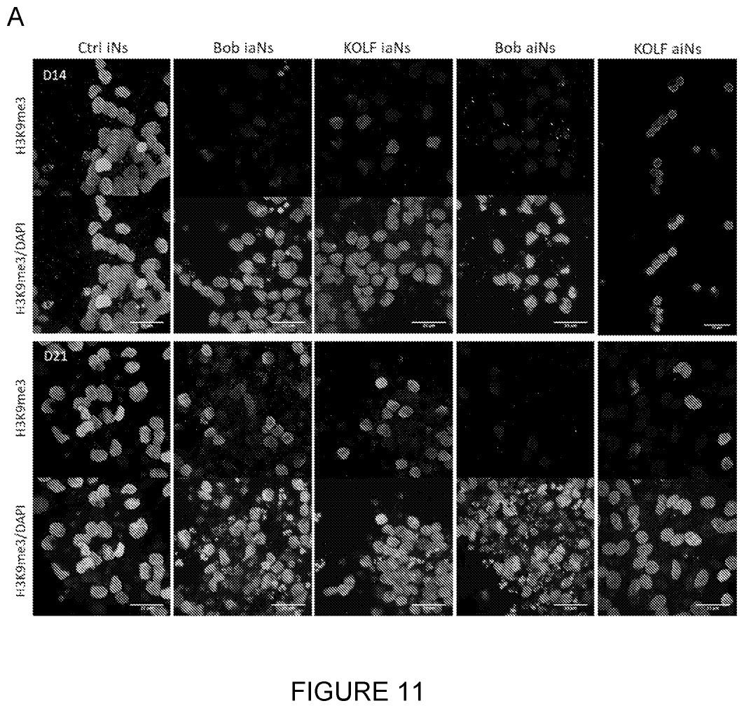

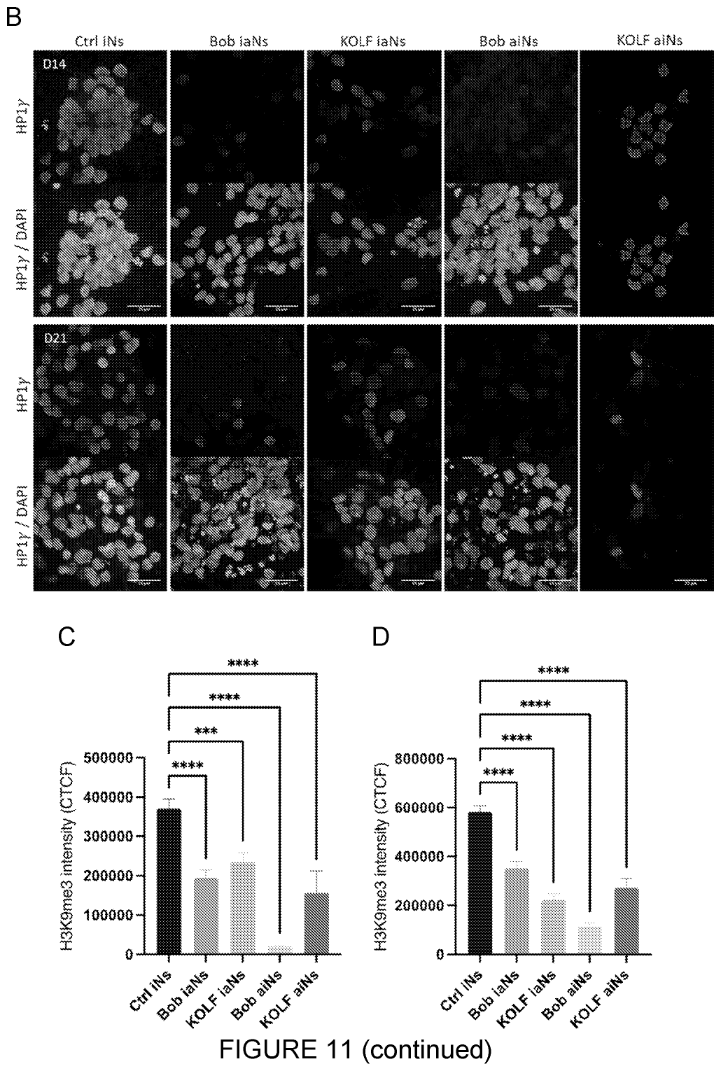

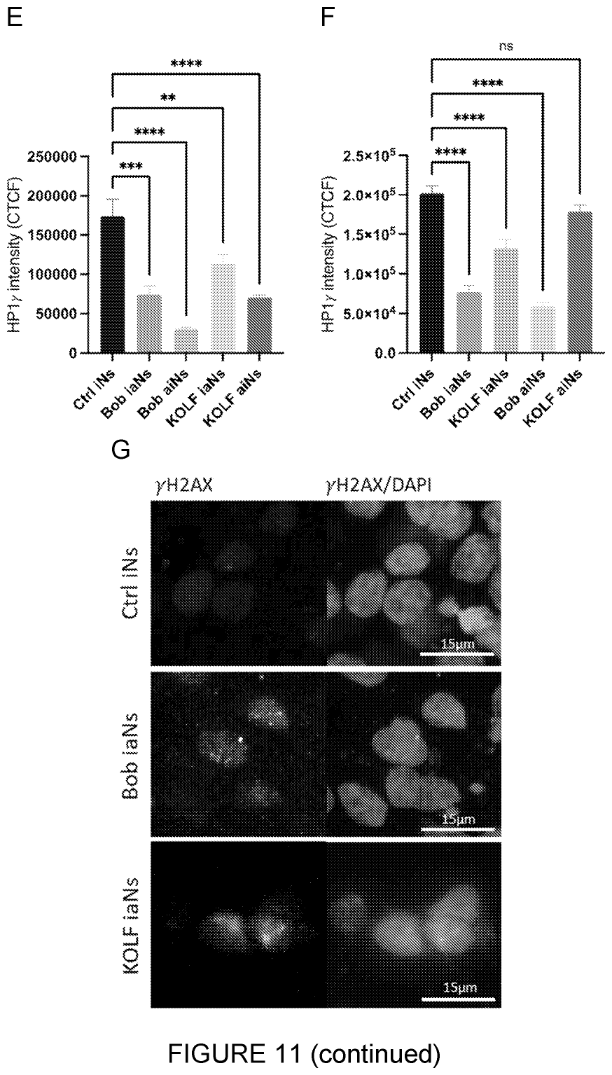

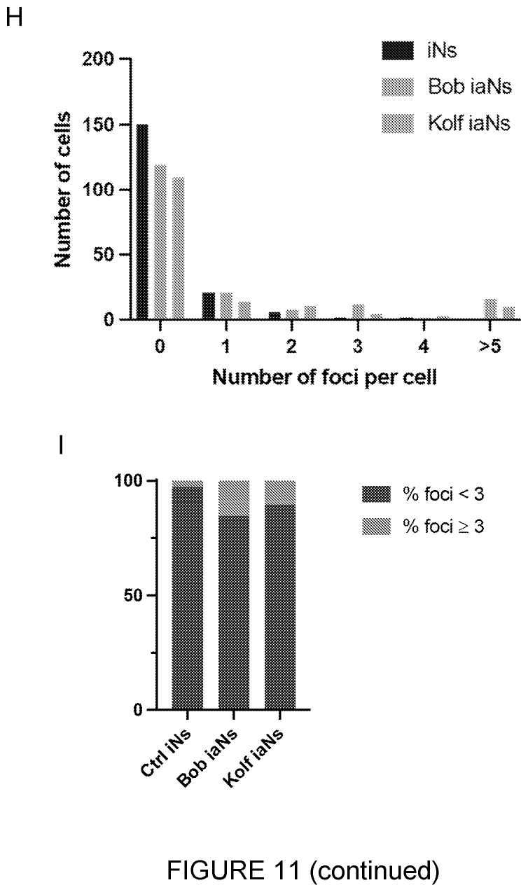

To further characterise the age-like phenotype of the iNs following progerin overexpression, the expression of two heterochromatin markers, H3K9me3 and HP1γ were assessed. Remarkably, both markers were significantly downregulated in the aged neurons at day 14 and day 21 post induction (FIGS. 11A-F). These age-like phenotypes have not been observed previously in neurons overexpressing progerin (Miller et al. (2013)). In addition, formation of DNA double-strand breaks, an important hallmark of physiological ageing, was observed in the aged neurons, based on γH2AX staining, where the aged iNs showed higher percentage of cells with foci >5 than the control non-aged iNs (FIG. 11G).

### Example 6—Mitochondrial Dysfunction in Aged iNeurons

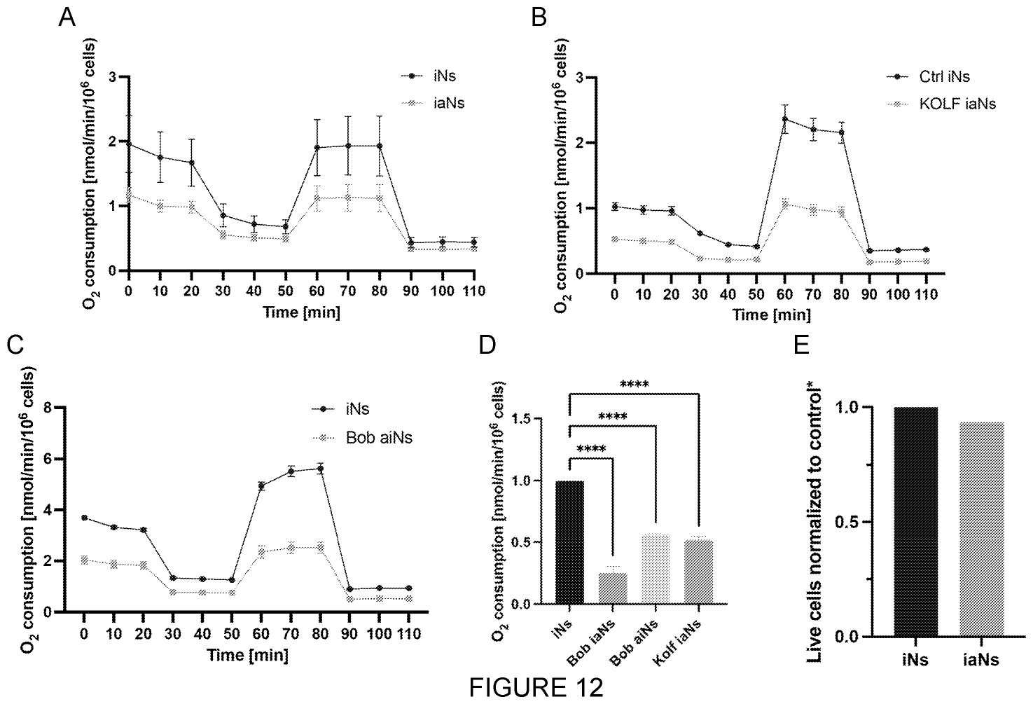

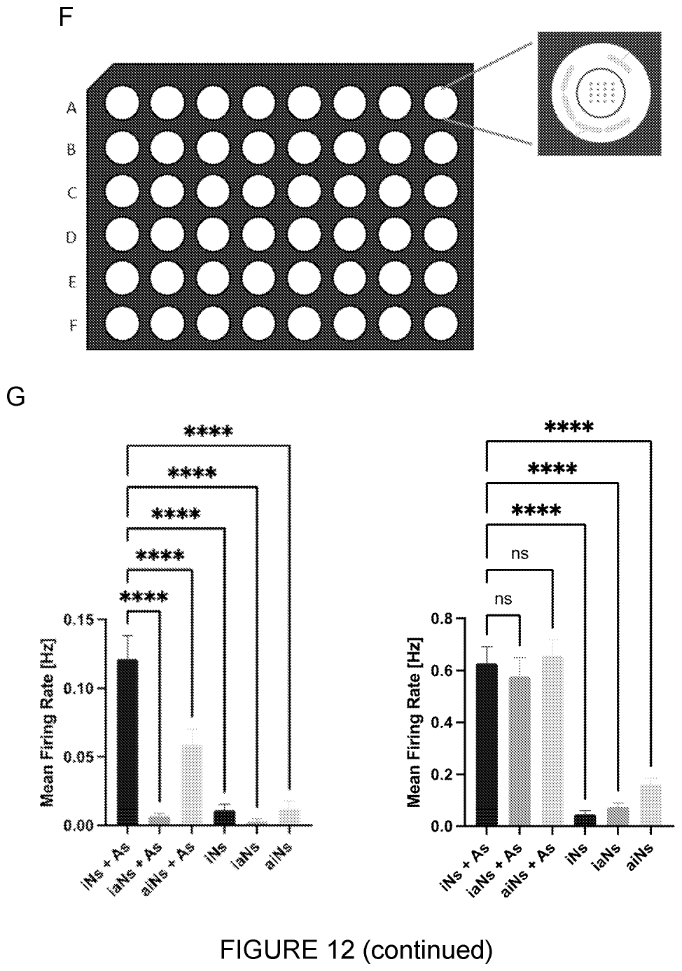

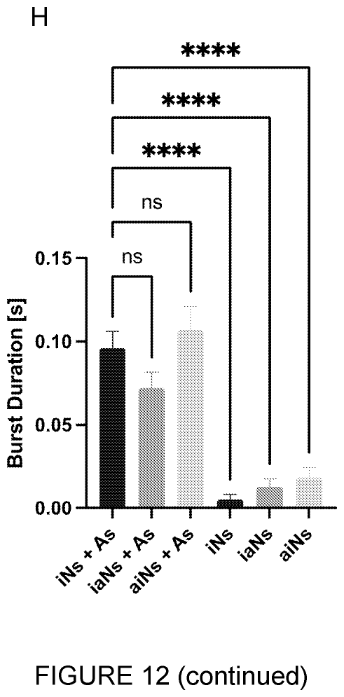

Mitochondrial dysfunction is a well-recognized hallmark of ageing, manifesting as reduced oxygen consumption. Building on the data showing changes in process formation and select ageing hallmarks, the mitochondrial activity in progerin-exposed hiNeurons was investigated using Seahorse Mito Stress Test assay. As with physiological ageing, it was found that progerin-exposed hiNeurons exhibited a reduction in basal and peak oxygen consumption for both pre- and post-aged iNeurons (FIGS. 12A-D). To rule out the possibility of a massive cell death leading to this reduction in oxygen consumption rates (OCRs), the viability of the cells was tested which confirmed the majority of cells were viable (FIG. 12E).

### Example 7—Electrophysiology Recordings in Aged iNeurons

To test the functionality of the aged iNs based on their electrophysiology properties, the cells were on multi electrode arrays with and without rodent astrocytes. At D14 post induction the aged iNs showed reduced activity compared to the non-aged iNs. However, by D21 the aged group activity returned to normal and displayed burst duration similarly as the non-aged group (FIGS. 12F-H). This shows that the neuronal network communication was indeed affected during the early stages of maturation but was able to reach normal levels at a later stage.

### Example 8—Transcriptomic Profiles of Aged hiPSCs and Aged iNeurons

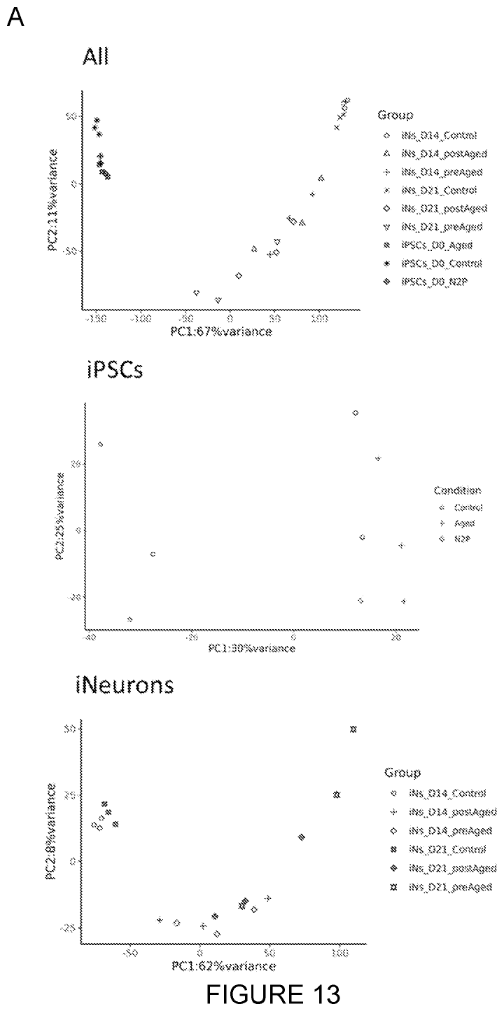

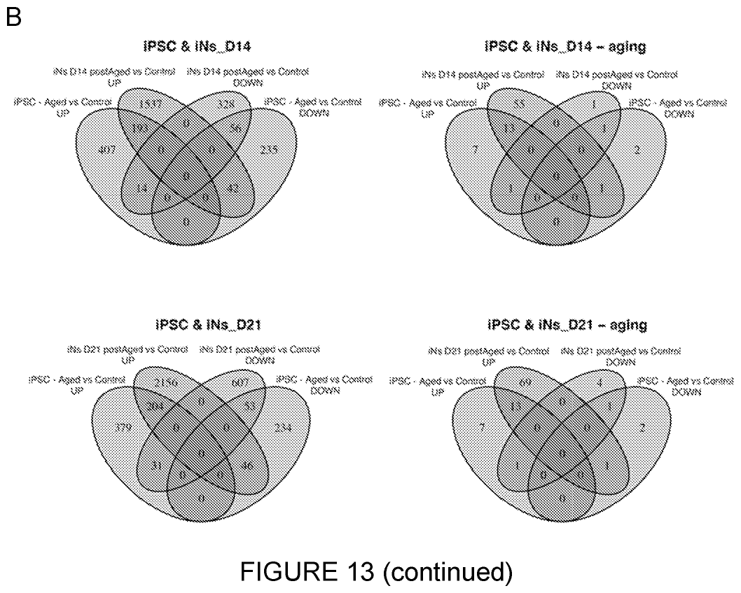

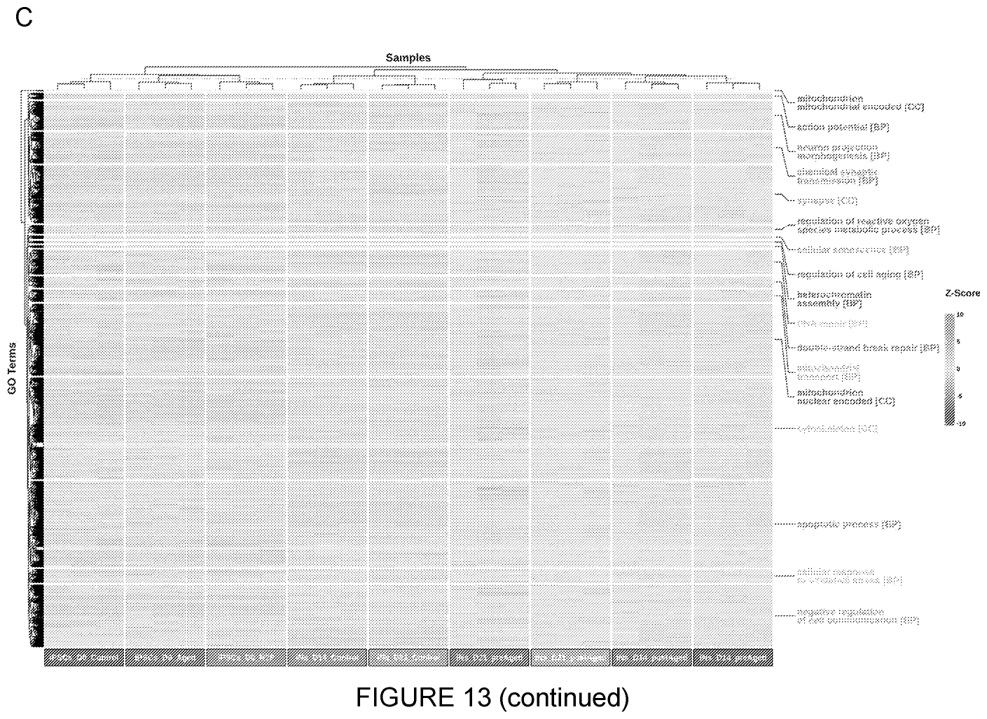

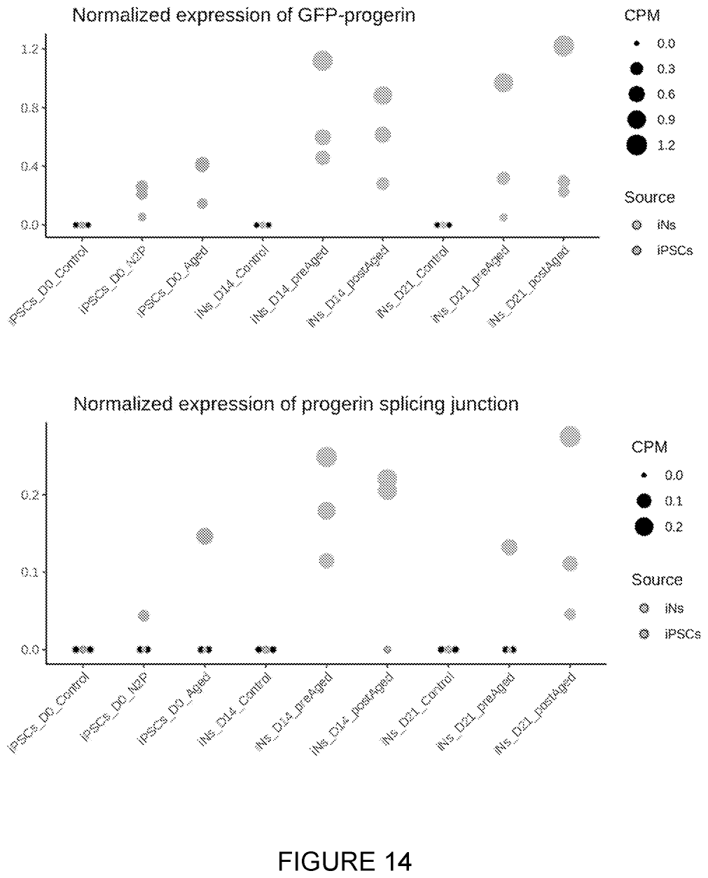

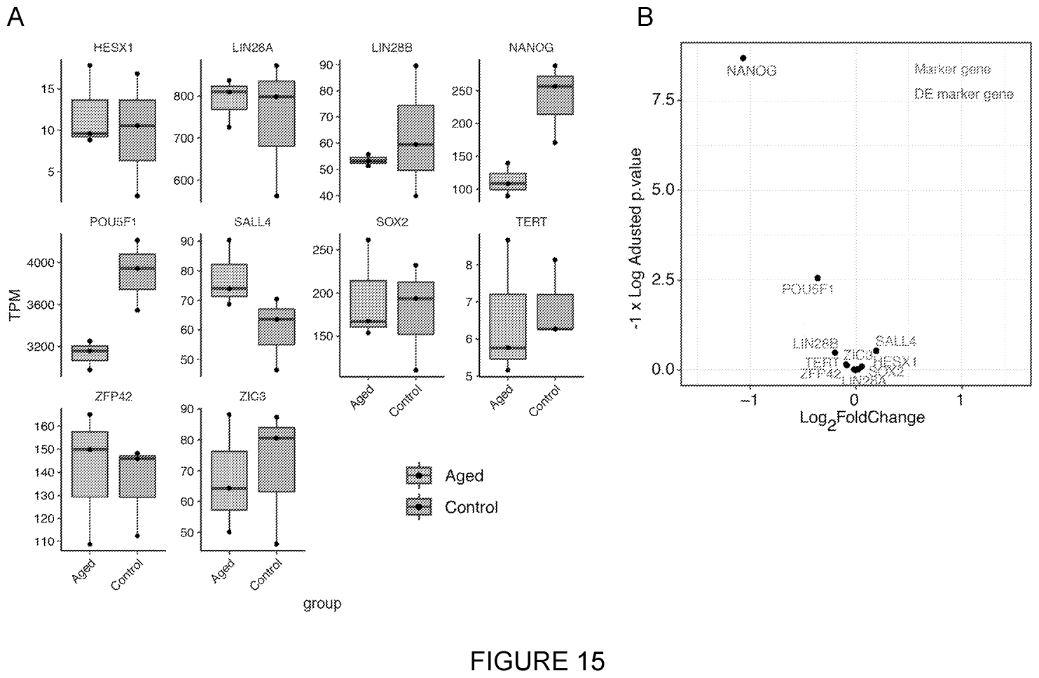

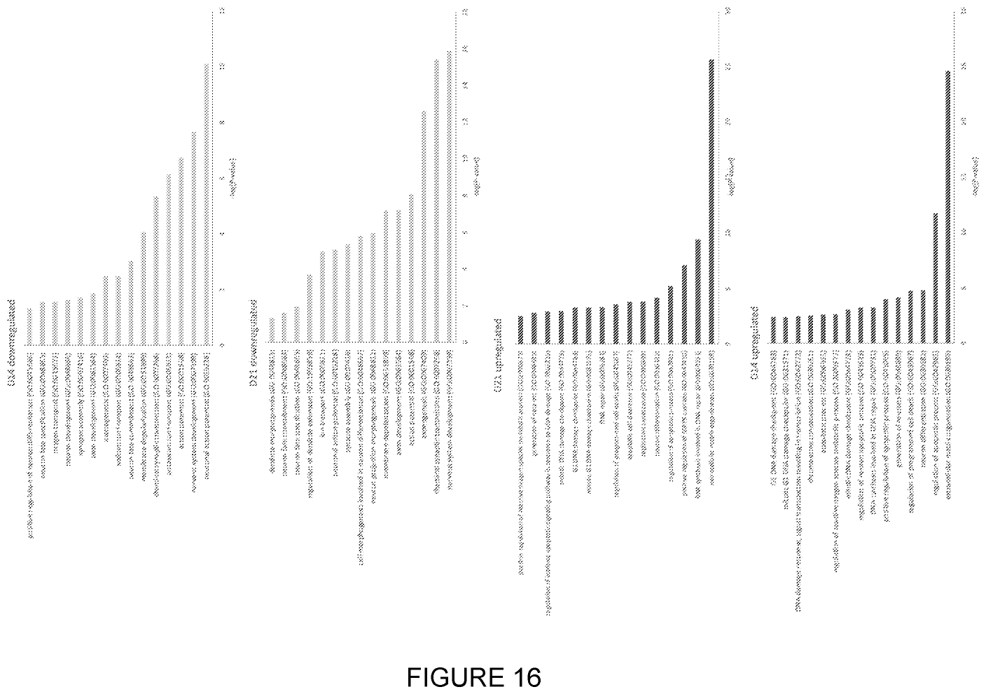

Next, the transcriptomic profiles of the aged iPSCs and iNs were investigated by performing bulk RNA-sequencing (RNA-seq) analysis. As controls iPSCs^{NGN2}, IPSCs^{N2P} and INs^{NGN2} untreated with cumate were used. Principal component analysis (PCA) demonstrated tight clustering of the control non-aged iPSCs group and iNs group while the aged groups were more sparse (FIG. 13A). Surprisingly, the control iPSCs^{N2P} clustered together with the aged iPSCs^{N2P}, which raised concerns that the cumate inducible system might be leaky. To verify this, the number of reads for progerin and GFP from the transcript data were analysed. It was found that indeed the cumate inducible system was leaky and that the control iPSCs^{N2P} showed expression of GFP-progerin even though the cells were not treated with cumate (FIG. 14). Next, the RNA-seq data between the different groups was compared to identify differentially expressed genes (DEGs). This allowed to find transcriptomic changes and to identify unique and common genes between the tested groups which are related to ageing. The comparison identified 2542 DEGs for the aged iNs at D14, out of which 84 were age-related (FIG. 13B). For aged iNs at D21, almost twice the number were identified, 5011 DEGs, out of which 122 were age-related (FIG. 13B). Of the age-related genes, the majority were upregulated: 79 and 110 for D14 and D21 respectively. For the aged iPSCs 25 aged-related and a total of 1031 DEGs were detected, among those DEGs were Nanog and OCT4 (POU5F1) that were both downregulated following overexpression of progerin (FIG. 15). To further support the ageing phenotype, gene ontology (GO) terms were investigated. It was found that the DEGs which were upregulated in the aged iNs at both D14 and D21 were associated with DNA damage, apoptosis, and senescence GO-terms, while those downregulated were associated with synapses, action potential, and neuronal development (FIGS. 13C and 16).

### Example 9—Epigenetic Changes in Aged hiPSCs and iNeurons

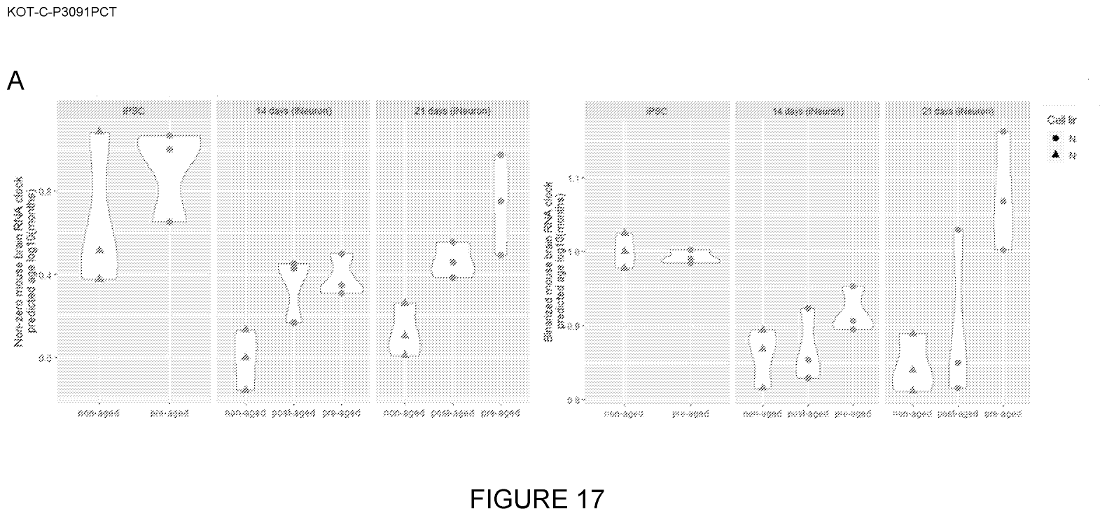

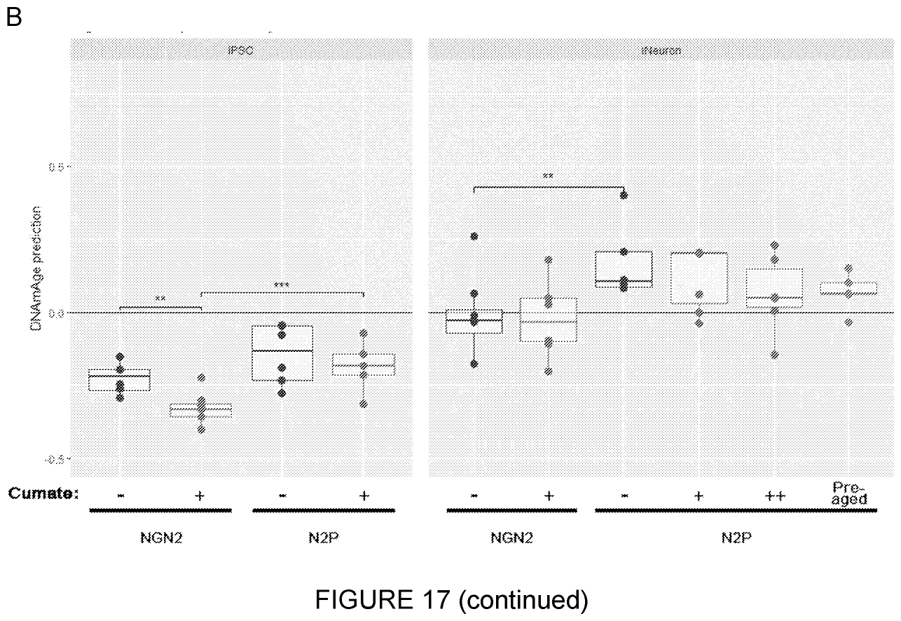

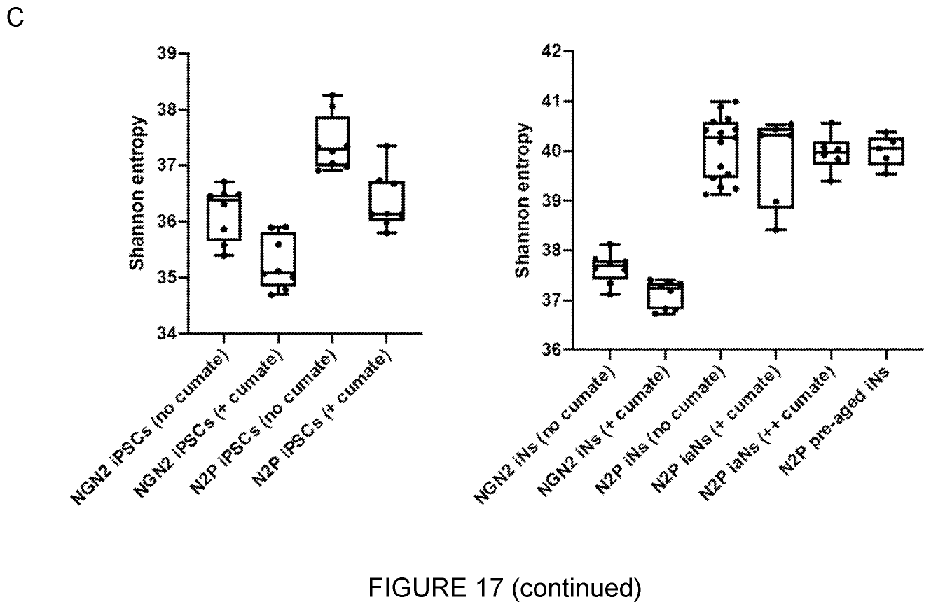

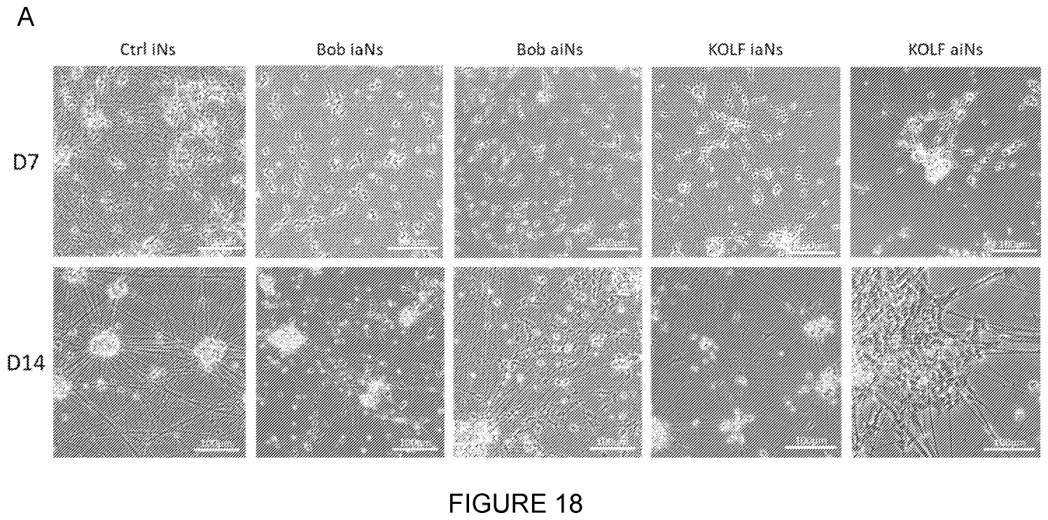

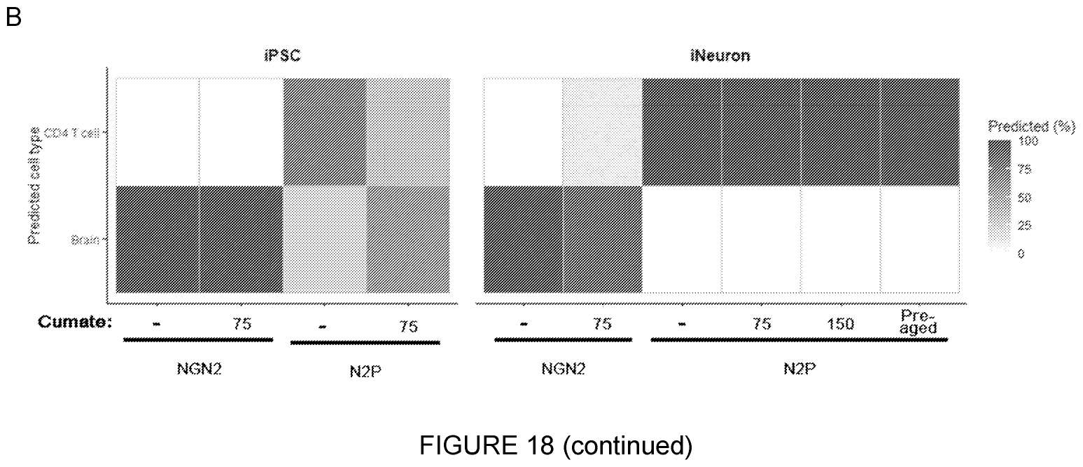

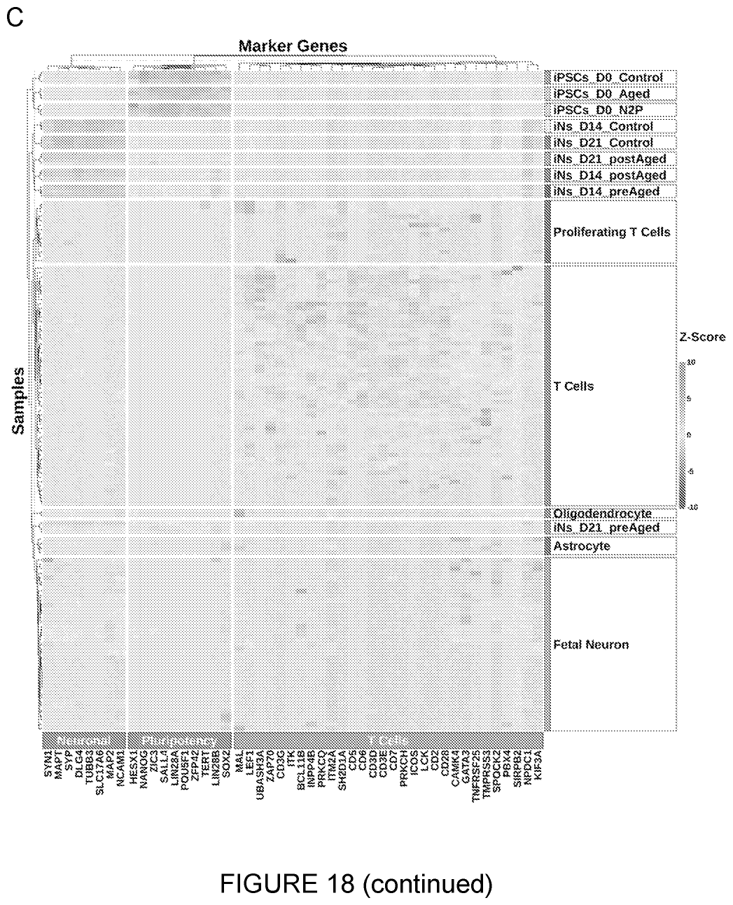

So far, this model system was able to capture physiological ageing phenotypes in both iPSCs and iNs. To test whether the epigenetic landscape of the cells was also affected by the artificial ageing, transcriptomic clock analyses were performed. These clocks are fitting algorithms which can provide an age estimation for the cells based on Tabula Muris Senis, a mouse brain atlas (Almanzar et al., 2020). These data showed that the control non-aged iNs slightly shifted to a more aged phenotype from D14 to D21. Remarkably, when progerin was induced, the ageing signature was significantly affected and both D14 and D21 were shifted to an older age (FIG. 17A). However, when trying to correlate the transcriptomic clock with the iPSCs group, the cells age could not be predicted. Next, the data was analysed based on a different epigenetic clock, the Horvath clock. This clock can predict the cells age based on CpG methylation sites in the DNA. Surprisingly, no significant changes in age were detected for the aged iPSCs and iNs (FIG. 17B). By using a modified version of this clock which correlates epigenetic changes to cell type, it was found that the aged cells are switching into a CD4 T cell identity (FIG. 18). The change of cell morphology was also observed in vitro for the aged group (FIG. 18). However, the transcriptomic profiles of the cells did not match those of CD4 T cells and were still resembling neuronal cells (FIG. 18), suggesting this might be due to a different mechanism. To further estimate the epigenetic changes, Shannon entropy was measured based on the number of methylated CpG sites. This relatively new approach can predict the chronological age of the cells as the entropy increases with age, which also means the epigenome loses information content (de Martin-Herranz Genome Biology 2019). Indeed, it was found that both the aged iPSCs and aged iNs had a higher entropy than the control groups (FIG. 17C). It should be noted that iPSCs_{N2P} and iNs_{N2P} controls were also shown to age, this might be due to the leak in the cumate inducible system that was observed previously.

### Example 10—Engineering and Functionality Testing of CRISPR Screening Line

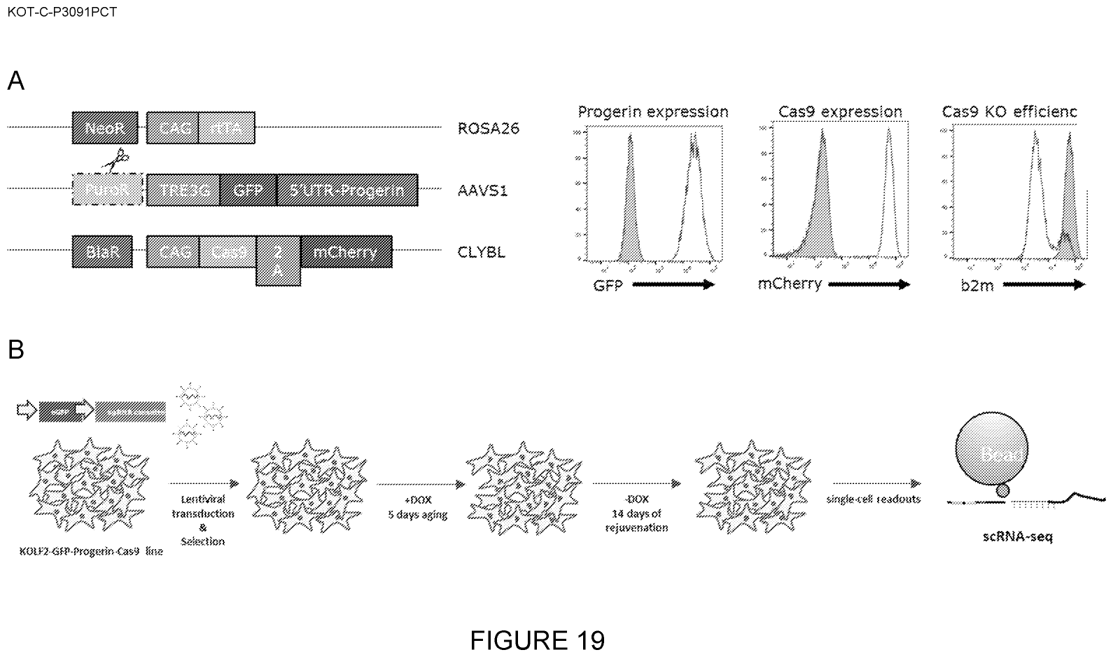

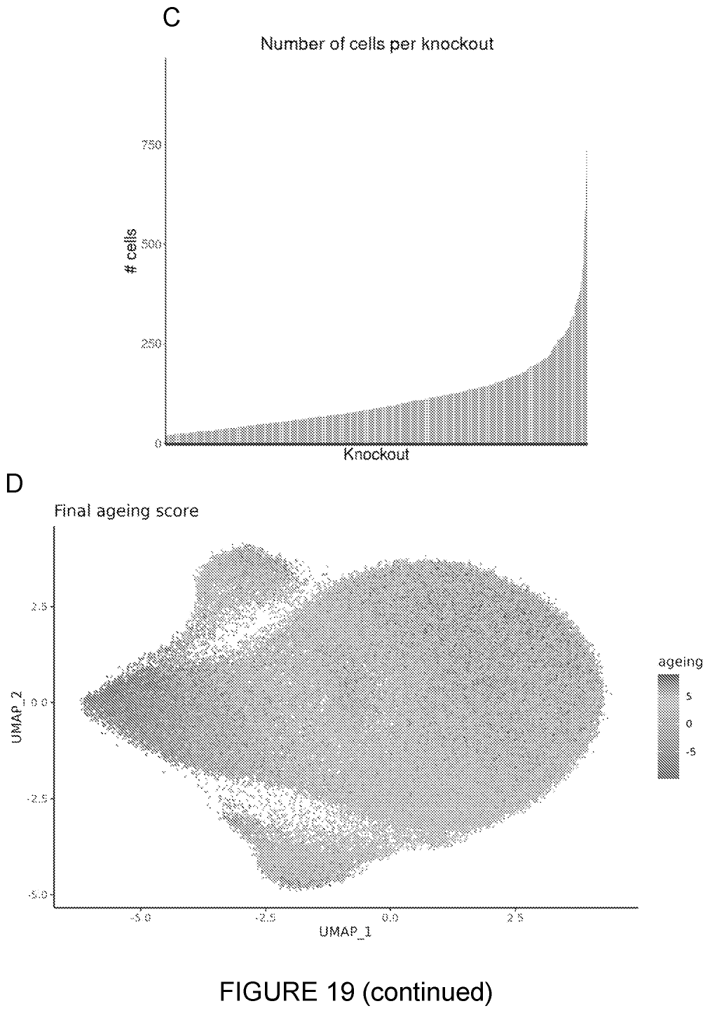

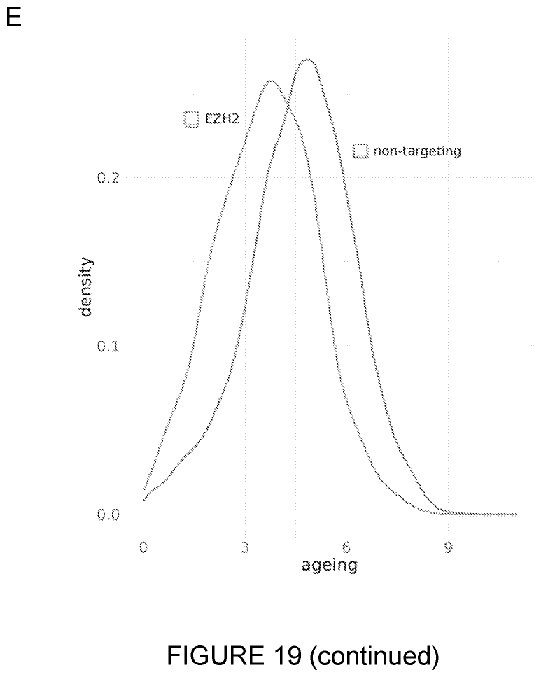

In order to generate a progerin-based ageing model for CRISPR screening, a human iPSC line was created which (i) can be artificially aged by the overexpression of progerin, and (ii) expresses the Cas9 protein in a constitutive manner (FIG. 19A). Expression of the Progerin and Cas9 cassette was confirmed by analysis of GFP (marking Progerin) and mCherry (marking Cas9) activity, respectively. In case of GFP overexpression (and thus progerin), cells were treated for 5 days with doxycycline to ensure stable expression of the transgene. To benchmark the expression and activity of Cas9, the engineered iPSC line was transduced with a lentiviral vector encoding sgRNA targeting the 32 microglobulin (B2M) gene (GGCCGAGATGTCTCGCTCCGGTTTCAGAGCTATGCTGGAAACAGCATAGCAAGTTGAA ATAAGGCTAGTCCGTTATCAACTTGAAAAAGTGGCACCGAGTCGGTGC) which is expressed on all nucleated cells. The complex of Cas9 and sgRNA will disrupt the expression of the B2M protein by introducing double-strand breaks in its genomic region, resulting in insertions or deletions (indels) and thus disrupting the open reading-frame of B2M. Loss of B2M protein expression was observed upon delivery of the specific sgRNA compared to an untreated control, thus confirming Cas9 activity in this cell line (FIG. 19A).

A large-scale CRISPR knock-out (KO) screen was performed, targeting 1,000 genes that are possibly involved in ageing and related processes (rejuvenation and senescence) (FIGS. 19B-C). Out of the 1000 candidate genes screened, 796 Knock-Outs with at least 20 cells were identified. Certain gene knockouts had an impact on cellular fitness, thus confirming the functionality of the CRISPR/Cas9 and sgRNA architecture. And allowing the identification of gene knockouts that influence the rejuvenation of iPSCs after Progerin-induced ageing at the transcriptional level.

Bulk RNA-sequencing for the KOLF2-GFP-Progerin-Cas9 line was performed to decipher the difference in gene expression between iPSCs that were treated for 5 days with doxycycline to induce progerin overexpression, compared to the same cells that were left untreated, thus deriving a transcriptomic ageing signature. To identify the influence of single gene knockouts on the ageing signature of iPSCs at a single-cell level, a linear discriminant analysis (LDA) was performed to create an “ageing scale”. That is, the ageing signature was used to create a continuous variable (i.e. the ageing score) describing how “aged” a single cell is on a transcriptional level. This ageing scale was integrated into a Uniform Manifold Approximation and Projection (UMAP) plot of all single cells processed within the CRISPR knockout screen (FIG. 19D). This scale was used to evaluate the effect of knockouts on the aging/rejuvenation process compared to cells transduced with a non-targeting sgRNA (“non-targeting”) that served as a control.

Several knockouts were identified that had an influence on the ageing/rejuvenation of iPSC. These effects were either (i) pro-ageing (i.e. knockout cells become confined in ageing state) or (ii) anti-ageing (i.e. knockout cells ameliorate the effect of progerin on gene expression). In this example, the knockout of EZH2 had an anti-ageing effect in the LDA, since cells harbouring the respective knockout were less “aged” compared to control conditions (FIG. 19E). EZH2 has been linked to cellular senescence, a process in which cells stop dividing and enter a state of permanent growth arrest, which is a hallmark of ageing (Ito et al. (2018) Cell Reports). Additionally, EZH2 is reported to be part of the protein machinery that shapes the ageing epigenome (Mozhui et al. (2017) Mech Ageing Dev). Overall, these results indicate that utilizing the CROP-sequencing screening platform along with the progerin-based ageing model is a valid approach for pinpointing genes involved in the process of ageing and rejuvenation.

1. A method of transiently inducing ageing in a stem cell, said method comprising:
(i) introducing one or more ageing-inducing factor into the cell or exposing the cell to one or more exogenous ageing-inducing factor for a period of time, and
(ii) removing or reducing the one or more ageing-inducing factor, thereby leading to a reversal of one or more ageing phenotype in the cell.

2. The method of claim 1, wherein the stem cell is a pluripotent stem cell, an induced pluripotent stem cell (iPSC), a germline stem cell, a multipotent stem cell, an oligopotent stem cell, a unipotent stem cell or a tissue-resident stem cell.

3. A method of transiently inducing ageing in a somatic cell derived from an induced pluripotent stem cell (iPSC) by forward programming, said method comprising:
(i) introducing one or more ageing-inducing factor into the cell or exposing the cell to one or more exogenous ageing-inducing factor for a period of time prior to or after forward programming of the iPSC, and
(ii) removing or reducing the one or more ageing-inducing factor, thereby leading to a reversal of one or more ageing phenotype in the cell.

4. The method of claim 3, wherein the somatic cell is a neuron, glia cell, blood cell, immune cell, liver cell, muscle cell, myocyte, fibroblast, skin cell, bone cell, cartilage cell, epithelial cell, endothelial cell or adipocyte.

5. The method of any one of claims 1 to 4, wherein introducing one or more ageing-inducing factor in step (i) comprises one or more expression cassette comprising a sequence encoding the one or more ageing-inducing factor, optionally wherein the expression cassette further comprises an inducible promoter which is regulated by a transcriptional regulator protein.

6. The method of claim 5, wherein the expression cassette and a gene encoding a transcriptional regulator protein are inserted into a genetic safe harbour site,
wherein the expression cassette further comprises an inducible promoter which is regulated by the transcriptional regulator protein.

7. The method of claim 5, wherein a gene encoding a transcriptional regulator protein is inserted into a first genetic safe harbour site and the expression cassette is inserted into a second genetic safe harbour site,
wherein the expression cassette further comprises an inducible promoter which is regulated by the transcriptional regulator protein, and
wherein the first and second genetic safe harbour sites are different.

8. The method of any one of claims 5 to 7, wherein expression of the sequence encoding the ageing-inducing factor or the activity of the transcriptional regulator protein is controlled by an exogenously supplied substance, such as tetracycline and derivatives thereof or cumate.

9. The method of any one of claims 1 to 8, wherein removing or reducing the one or more ageing-inducing factor in step (ii) comprises active removal or reduction, thereby leading to the reversal of one or more ageing phenotype in the cell.

10. The method of any one of claims 5 to 9, wherein expression of the sequence encoding the ageing-inducing factor or the activity of the transcriptional regulator protein is reduced and/or inactivated by the removal of an exogenously supplied substance, such as tetracycline and derivatives thereof or cumate.

11. The method of any one of claims 1 to 10, wherein the one or more ageing-inducing factor is an alternative splice form of Lamin A, such as progerin, and/or
wherein the one or more ageing-inducing factor is a mitochondrial DNA mutation, such as deletion of DNA polymerase subunit gamma (POLG), and/or
wherein the one or more ageing-inducing factor is an ageing-inducing compound, such as a telomerase inhibitor, reactive oxygen species (ROS) or a compound that disrupts or stresses mitochondria function, such as 6-hydroxydopamine (6-OHDA), valinomycin, CCCP, rotenone or hydrogen peroxide, and/or
wherein the one or more ageing-inducing factor is an inducer of DNA damage, such as ionizing radiation, platinum-containing drugs, cyclophosphamide, chlorambucil or temozolomide.

12. An artificially aged stem cell or somatic cell derived from an iPSC by forward programming produced by the method of any one of claims 1 to 11.

13. Use of the artificially aged stem cell and/or somatic cell derived from an iPSC by forward programming of claim 12 in a method of identifying a gene or a combination of genes involved in the reversal of an ageing phenotype or in the maintenance of a non-aged phenotype in the cell.

14. A method of identifying a gene or a combination of genes involved in the reversal of an ageing phenotype or in the maintenance of a non-aged phenotype in a cell, said method comprising the steps of:
i) introducing one or more ageing-inducing factor into the cell or exposing the cell to one or more exogenous ageing-inducing factor for a period of time;
ii) optionally performing a loss-of-function, inhibitory, knock-out, gain-of-function or combinatorial screen in the cell;
iii) measuring an ageing phenotype of the cell; and
iv) identifying a gene or a combination of genes as being involved in the reversal of an ageing phenotype or in the maintenance of a non-aged phenotype when the ageing phenotype is altered and/or its progression is altered in the cell when a loss-of-function, inhibitory, knock-out, gain-of-function or combinatorial screen is performed compared to when the cell is not subjected to a screen.

15. The method of claim 14, wherein introducing one or more ageing-inducing factor into the cell or exposing the cell to one or more exogenous ageing-inducing factor for a period of time in step (i) comprises introducing one or more expression cassette comprising a sequence encoding said one or more ageing-inducing factor and an inducible promoter which is regulated by a transcriptional regulator protein into the cell,
wherein expression of the sequence encoding the ageing-inducing factor or the activity of the transcriptional regulator protein is controlled by an exogenously supplied substance, such as tetracycline and derivatives thereof or cumate.

16. The method of claim 14 or claim 15, wherein the loss-of-function, inhibitory, knock-out, gain-of-function or combinatorial screen comprises a whole-genome screen, and/or
wherein the loss-of-function, inhibitory, knock-out, gain-of-function or combinatorial screen is performed using CRISPR.

17. The method of claim 16, wherein the screen is a loss-of-function or inhibitory screen and the CRISPR is CRISPRi, a knock-out screen and the CRISPR is CRISPR-ko, a gain-of-function screen and the CRISPR is CRISPRa, or a combinatorial screen and the CRISPR is a combination of two or more of: CRISPRi, CRISPR-ko and CRISPRa.

18. The use of claim 13 or the method of any one of claims 14 to 17, wherein the ageing phenotype of the cell is selected from one or more of: proliferation, senescence, changes in cell and/or nuclear morphology, mitochondrial function, changes in gene expression, upregulation of Lamin-A and/or Lamin-C nuclear protein, epigenetic marks associated with ageing, altered DNA or histone methylation, methylation entropy, DNA double strand breaks, telomere length, and a transcriptomic and/or epigenetic clock, in particular a transcriptomic clock, such as a transcriptomic signature associated with an aged phenotype.

19. The use or method of claim 18, wherein the ageing phenotype is reduced proliferation and/or increased senescence, and/or
wherein the changes in nuclear morphology are folding abnormalities, blebbing and/or loss of nuclear circularity, and/or
wherein the mitochondrial function is reduced oxygen consumption and/or increased mitochondrial reactive oxygen species (ROS), and/or
wherein the changes in gene expression are selected from one or more of: downregulation of somatic cell lineage identity genes, downregulation of mitochondrial genes, upregulation of apoptosis- and/or senescence-related genes, and upregulation of DNA damage-related genes, and/or
wherein the epigenetic marks associated with ageing are selected from one or more of: reduced heterochromatin trimethylated H3K9 (H3K9me3), reduced HP1γ, and increased γH2AX, and/or
wherein the telomere length is shortened, and/or
wherein the transcriptomic and/or epigenetic clock is a single cell transcriptomic and/or epigenetic clock, in particular a single cell transcriptomic clock, such as a transcriptomic signature associated with an aged phenotype.

20. The use or method of any one of claims 13 to 19, wherein the cell is a neuron derived from an iPSC by forward programming, and optionally wherein the ageing phenotype is a phenotype of a neurological degenerative disease, such as reduced process density and/or connectivity, reduced average dendritic length, reduced neurite diameter, downregulation of neuronal marker genes or reduced electrophysiological activity.

21. A method of rejuvenating a somatic cell comprising altering the expression and/or activity of one or more gene identified by the use or method of any one of claims 13 to 20.

22. The method of claim 21, wherein the rejuvenated somatic cell comprises a reduced and/or slowed progression of an ageing phenotype compared to a non-rejuvenated somatic cell, and wherein the ageing phenotype is as defined in claim 18 or claim 19.

23. The method or use of any one of claims 1 to 11 or claims 13 to 22, wherein the method or use is performed in vitro.
